(19) United States
(12) Patent Application Publication
(10) Pub. No.: US 2018/0147231 A1
(43) Pub. Date: May 31, 2018

(54) METHOD FOR TREATMENT AND PROGNOSIS OF COLORECTAL CANCER

(71) Applicant: Oxford University Innovation Limited, Oxford (GB)

(72) Inventors: Sarah McCuaig, Oxford (GB); Fiona Margaret Powrie, Oxford (GB); Nathaniel Richard West, Oxford (GB)

(21) Appl. No.: 15/575,218

(22) PCT Filed: May 20, 2016
(86) PCT No.: PCT/GB2016/051466

§ 371 Date: Nov. 17, 2017

(30) Foreign Application Priority Data
May 22, 2015 (GB) 1508841.2

(51) Int. Cl. A61K 31/713; C12Q 1/6886; A61K 45/06; A61P 35/00; G01N 33/574; A61K 39/00

(52) U.S. Cl. CPC A61K 31/713; C12Q 1/6886; A61K 45/06; G01N 2800/52; G01N 33/57419; A61K 2039/505; G01N 2333/7155; A61P 35/00

(57) ABSTRACT

The invention relates to the treatment and prognosis of colorectal cancer, especially proximal colorectal cancer. It also relates to identifying patients with colorectal cancer who are likely to respond to therapy with an inhibitor of interleukin 22 signalling.

### FIELD OF THE INVENTION

The invention relates to the treatment and prognosis of colorectal cancer, especially proximal colorectal cancer. It also relates to identifying patients with colorectal cancer who are likely to respond to therapy with an inhibitor of interleukin 22 signalling.

### BACKGROUND OF THE INVENTION

Chronic intestinal inflammation is a well-known risk factor for colorectal cancer (CRC).¹ Sporadic CRCs that do not arise in a colitic context also elicit inflammatory responses.² Once thought to be exclusively involved in immune surveillance and antitumor immunity, intratumoral leukocytes are now understood to also have pro-tumorigenic roles. As such, inflammation is regarded as an enabling characteristic for the acquisition of the core hallmarks of cancer.² Leukocyte-derived cytokines that modulate cancer cell proliferation, survival, and dissemination are central linchpins in this relationship.³

Interleukin 22 (IL-22) is an IL-10 cytokine superfamily member secreted by CD4⁺ T cells and innate lymphoid cells in the tumor microenvironment. IL-22 plays a critical role in intestinal epithelial repair, but is also indispensable for primary intestinal tumorigenesis in murine models. For example, IL-22 blockade attenuates experimental colitis-driven tumorigenesis.⁴ Similarly, a pro-tumorigenic role for IL-22 was identified through manipulation of the established Apc^{Min/+} genetic model of CRC.⁵ Furthermore, IL-22 has been associated with human gastrointestinal cancer progression⁶ and may promote colorectal cancer stemness.⁷ Nevertheless, the clinical relevance of IL-22 signaling in human CRC remains unaddressed.

IL-22 signals through a heterodimeric receptor comprised of the IL-22 receptor alpha 1 (IL-22RA1) subunit and an IL-10 receptor B (IL-10RB) subunit, which is also utilized by several other members of the IL-10 family.^{8,9} Expression of IL-22RA1 is largely restricted to the epithelium of mucosal tissues, where it potently activates Janus kinases and signal transducer and activator of transcription 3 (STAT3). Although not as extensively characterized, IL-22 also activates mitogen activated protein kinase (MAPK) pathways, as well as the phosphatidylinositol-3-kinase (PI3K)/Akt cascade.^{10,11} Finally, IL-22 has been shown to activate NF-κB and, through synergism with STAT3, induce expression of genes involved in cell cycle progression and inhibition of apoptosis.¹²

While classified as an interleukin, IL-22 does not mediate direct cross-talk between leukocytes, but rather between leukocytes and non-hematopoietic cells, as receptor expression is restricted to the non-hematopoietic compartment. Signaling downstream of the IL-22 receptor is mediated predominately via JAK/STAT pathways. The majority of the well-documented physiologic and pathologic functions of IL-22 are STAT3-dependent. Atypically, the intracellular domain of IL-22R1 is constitutively associated with STAT3 allowing for rapid activation upon receptor dimerization by phosphorylation at both Tyr-705 and Ser-727.^{37,38} IL-22-mediated STAT3 signaling massively induces the expression of suppressor of cytokine signaling 3 (SOCS3), which inhibits signaling downstream of cytokine receptors containing a gp130 domain (ie. IL-6, IL-11). Interestingly, both subunits of the IL-22 receptor lack SOCS3 binding sites and thus the IL-22R is not subject to feedback inhibition by SOCS3.³⁹ Although not as extensively characterized, IL-22R engagement also activates several mitogen activated protein kinase (MAPK) pathways including p38 and extracellular signaling related kinase (ERK). IL-22-induced phosphatidylinositol-3-kinase (PI3K) activation is required for migration of colonic epithelial cells and Akt activation via IL-22 enables normal proliferation of human epithelial keratinocytes and inhibits apoptosis in renal tubular epithelial cells.^{40,41} Finally, IL-22 has been shown to activate NF-κB and through synergism with STAT3 induce expression of genes involved in cell cycle progression and inhibition of apoptosis.³⁷ Therefore IL-22 is a pleiotropic cytokine that can activate multiple signaling pathways and as such requires careful regulation. An important component of IL-22 regulation is the IL-22 neutralizing receptor IL-22-binding protein (IL-22BP or IL-22Rα2), a soluble receptor produced by CD11c⁺ cells that sequesters IL-22 and prevents its activity.⁴² The existence of the IL-22BP, in evolutionary terms, underscores the necessity for tight regulation of this pathway.

A major Ras isoform, KRAS, displays activating mutations in 40-45% of colorectal cancers, which are associated with resistance to EGFR-targeted therapy and some standard chemotherapies.¹³⁻¹⁵

### SUMMARY OF THE INVENTION

The inventors have surprisingly shown that patients whose colorectal cancer has both a KRAS mutation and a high amount of interleukin 22 receptor have a worsened prognosis relative to KRAS wild type or interleukin 22 receptor-low counterparts. Those patients having a proximal colorectal cancer with both a KRAS mutation and a high amount of interleukin 22 receptor have a dramatically worsened prognosis relative to KRAS wild type or interleukin 22 receptor-low counterparts. In addition, the inventors have also surprisingly shown that inhibitors of interleukin 22 signalling may be used to treat colorectal cancer, especially proximal colorectal cancer, having both a KRAS mutation and a high amount of interleukin 22 receptor.

The invention therefore provides a method of treating in a patient colorectal cancer which comprises a KRAS mutation and a high amount of interleukin 22 (IL-22) receptor, the method comprising administering to the patient an inhibitor of IL-22 signalling and thereby treating the cancer.

The invention also provides:
a method of treating colorectal cancer in a patient, the method comprising (a) determining whether or not the cancer comprises a KRAS mutation and measuring the amount of IL-22 receptor in the cancer and (b), if the cancer comprises a KRAS mutation and a high amount of IL-22 receptor, administering to the patient an inhibitor of IL-22 signalling and thereby treating the cancer;
an inhibitor of IL-22 signalling for use in a method of treating in a patient colorectal cancer which comprises a KRAS mutation and a high amount of IL-22 receptor;
use of an inhibitor of IL-22 signalling in the manufacture of a medicament for treating in a patient colorectal cancer which comprises a KRAS mutation and a high amount of IL-22 receptor;
a kit for treating colorectal cancer comprising (a) means for testing whether or not the cancer comprises a KRAS mutation and for measuring the amount of IL-22 receptor and (b) an inhibitor of IL-22 signalling;
a method for prognosing colorectal cancer in a patient, the method comprising determining whether or not the cancer comprises a KRAS mutation and measuring the amount of IL-22 receptor in the cancer, wherein the presence of a KRAS mutation and a high amount of IL-22 receptor in the cancer indicates that the patient has a worse prognosis than in the absence of a KRAS mutation and/or in the presence of a low amount of IL-22 receptor;
a method for determining whether or not a patient with colorectal cancer is likely to respond to therapy with an inhibitor of IL-22 signalling, the method comprising determining whether or not the cancer comprises a KRAS mutation and measuring the amount of IL-22 receptor in the cancer, wherein the presence of a KRAS mutation and a high amount of IL-22 receptor in the cancer indicates that the patient is likely to respond to therapy with an inhibitor of IL-22 signalling;
an in vitro assay for determining whether or not a patient with colorectal cancer is likely to respond to therapy with an inhibitor of IL-22 signalling, the assay comprising determining whether or not a sample from the cancer comprises a KRAS mutation and measuring the amount of IL-22 receptor in the cancer, wherein the presence of a KRAS mutation and a high amount of IL-22 receptor in the sample indicates that the patient is likely to respond to therapy with an inhibitor of IL-22 signalling;
an in vitro assay for prognosing colorectal cancer in a patient, the assay comprising determining whether or not a sample from the cancer comprises a KRAS mutation and measuring the amount of IL-22 receptor in the cancer, wherein the presence of a KRAS mutation and a high amount of IL-22 receptor in the cancer indicates that the patient has a worse prognosis than in the absence of a KRAS mutation and/or in the presence of a low amount of IL-22 receptor;
a system for determining whether or not a patient with colorectal cancer is likely to respond to therapy with an inhibitor of IL-22 signalling, the system comprising (a) a measuring module for determining whether or not the cancer comprises a KRAS mutation and for measuring the amount of IL-22 receptor in the cancer, (b) a storage module configured to store control data and output data from the measuring module, (c) a computation module configured to provide a comparison between the value of the output data from the measuring module and the control data; and (d) an output module configured to display whether or not the patient is likely to respond to therapy with an inhibitor of IL-22 signalling based on the comparison, wherein the presence of a KRAS mutation and a high amount of IL-22 receptor in the cancer indicates that the patient is likely to respond to therapy with an inhibitor of IL-22 signalling; and
a system for prognosing colorectal cancer in a patient, the system comprising (a) a measuring module for determining whether or not the cancer comprises a KRAS mutation and for measuring the amount of IL-22 receptor in the cancer, (b) a storage module configured to store control data and output data from the measuring module, (c) a computation module configured to provide a comparison between the value of the output data from the measuring module and the control data; and (d) an output module configured to display the patient's prognosis, wherein the presence of a KRAS mutation and a high amount of IL-22 receptor in the cancer indicates that the patient has a worse prognosis than in the absence of a KRAS mutation or in the presence of a low amount of IL-22 receptor.

### DESCRIPTION OF THE FIGURES

FIG. 1 shows that KRAS mutation dramatically worsens prognosis in patients with IL22RA1^{high} tumours. Relapse free and overall survival according to IL22RA1 expression level and KRAS mutation status in Stage II/III patients in the GSE39582 French Cohort estimated using Kaplan-Meier methods. (A) RFS and (B) OS in the total cohort based on IL22RA1 expression level. Tumoral IL22RA1 expression above the 67^{th} percentile in the total cohort was categorized as high based on ROC analysis. (C) RFS and (D) OS in the total cohort based upon KRAS mutation status. (E) RFS and (F) OS among IL22RA1-high patients based upon KRAS mutation status. (G) RFS and (H) OS among IL22RA1-low patients based upon KRAS mutation status.

FIG. 2 shows that KRAS mutation dramatically worsens prognosis in patients with IL10RB^{high} tumors. Relapse free and overall survival according to IL10RB expression level and KRAS mutation status in Stage II/III patients in the GSE39582 French Cohort was estimated using Kaplan-Meier methods. (A) RFS and (B) OS in the total cohort based on IL10RB expression level. Tumoral IL10RB expression above the 67^{th} percentile in the total cohort was categorized as high. (C) RFS and (D) OS among IL10RB-high patients based upon KRAS mutation status. (E) RFS and (F) OS among IL10RB-low patients based upon KRAS mutation status.

FIG. 3 shows that inflammation metagene signatures are enriched in proximal versus distal CRCs (GSE39582). Analysis of immune cell subsets defined by metagene signatures in proximal versus distal CRCs in the GSE39582 cohort. Log₂ expression values display immune metagene signature enrichment. *p<0.05, **p<0.01, ***p<0.001, ****p<0.0001, unpaired two-tailed Mann-Whitney U test.

FIG. 4 shows that KRAS mutation is prognostic in IL22RA1^{high} patients in proximal but not distal CRC. Relapse free and overall survival according to IL22RA1 expression level and KRAS mutation status in Stage II/III patients in the GSE39582 French Cohort estimated using Kaplan-Meier methods. (A) RFS and (B) OS among IL22RA1-high patients with proximal tumors based upon KRAS mutation status. (C) RFS and (D) OS among IL22RA1-high patients with distal tumors based upon KRAS mutation status. (E) Relative proportions of stage II/III CRC patients in the GSE39582 cohort whose tumors were categorized as IL22RA1-high or IL22RA1-low, KRAS wild type or mutant, and proximal or distal.

FIG. 5 shows the distribution of IL22RA1 expression in combined GSE39582, PETACC3, TCGA datasets (n=2332).

FIG. 6 shows that IL-22RA1 is differentially expressed in colorectal tumours. Representative images of IL-22RA1 immunohistochemical analysis of two CRC tumors and corresponding normal adjacent tissue.

FIG. 7 shows the characterization of IL-22 signaling in six KRAS-WT and KRAS-mutant colorectal cancer cell lines. (A) qPCR analysis of IL22RA1 expression level on 3 KRAS-WT (Colo205, LS103, SW948) and 3 KRAS-mutant (T84, SW480, HCT116) CRC cell lines. n=3 *p<0.05, **p<0.01, one-way ANOVA with Tukey's post test for multiple comparisons. (B) Representative FACS plots of IL-22RA1 expression on the Colo205 (KRAS-WT, IL-22RA1^{high}), T84 (KRAS-Mut, IL-22RA1^{high}), and SW480 (KRAS-mutant, IL-22RA1^{low}) lines. (C) Western blot analysis of activation of STAT3, ERK, and Akt signaling pathways in 6 CRC lines following 24 h stimulation with 1 ng/mL IL-22, 10 ng/mL IL-22, and 1 ng/mL IL-6. 1 blot representative of 3 independent experiments. (D) qPCR confirmation of active downstream IL-22 signaling in CRC lines based on upregulation of SOCS3. n=3, *p<0.05, **p<0.01, ***p<0.001, ****p<0.0001, one-way ANOVA with Dunnett's post test for multiple comparisons.

FIG. 8 shows that IL-22 protects against oxaliplatin and 5 fluorouracil mediated cell death in KRAS-mutant, IL22RA1^{high} T84 cells. (A) MTT assay on Colo205, T84, and SW480 cells pre-treated or not pre-treated for 48 h with 10 ng/mL IL-22, then treated with 50 μM oxaliplatin or 5-FU for 48 h. MTT was added 2 h prior to end of 48 h incubation. Formazan particles were dissolved in DMSO and absorbance was measured at 540 nm. Raw absorbances are blank corrected, normalized to a no treatment or IL-22 only, and represented as a % reduction in viability compared to the no treatment or IL-22 only conditions. n=3 independent experiments with 3 experimental replicates, *p<0.05, ****p<0.0001, one-way ANOVA with Tukey's post test for multiple comparisons.

FIG. 9 shows that IL-22 enhances clonogenic outgrowth of KRAS-mutant, IL22RA1^{high} T84 CRC cells. (A) Schematic representation of primary sphere forming assay workflow. Cells were pre-treated with 10 ng/mL IL-22 for 48 hours, filtered to single cells and 1000 cells/well were seeded into 96 well low-binding plates in serum-free media containing 1% methylcellulose, 20 ng/mL EGF, 20 ng/mL bFGF with and without 10 ng/mL IL-22. Four experimental replicates were seeded for each condition. Cultured spheres from (B) Colo205, T84 and SW480 cell lines for 6 days. (C) Bright field microscopy images (4×) of single wells 6 days after seeding (1 experiment representative of 4). (D) MTT assay to assess viability of spheres 6 days after seeding (n=3). (E) Bright field microscopy of T84 spheres (20×) 6 days after seeding. (F) Quantification of spheres after 6 days of culture using ImageJ. Data represent mean+/−SD of 3 independent experiments, each with 3 technical replicates per condition. *p<0.05, one-way ANOVA with Dunnett's post test for multiple comparisons.

FIG. 10 shows that the protumourigenic effect of IL-22 is KRAS-dependent by using an isogenic pair of DLD-1 colorectal cancer cell lines in which the parental line (KRAS MUT) is a heterozygous KRAS G13D mutatant and a second line (KRAS WT) has been generated by adeno-associated viral knockout of the mutant KRAS allele. Therefore this isogenic pair differs only in KRAS mutation status and allows a clean system for comparison of KRAS-dependent IL-22 effects without inter-cell line mutational heterogeneity. (A) Representative FACS plots of IL-22RA1 expression and quantification of mean fluorescence intensity (WI) showing similar IL-22RA1 expression in the isogenic pair (4 experimental replicates). Phosflow analysis of intracellular signaling pathways activated by 30 min 10 ng/mL recombinant human IL-22 stimulation in the isogenic lines revealed (A) identical extent of phosphorylated STAT3 by IL-22 (B) differing basal level of phosphorylated ERK1/2 that was not IL-22 inducible but higher in the KRAS MUT line as expected and (C) IL-22 inducible phosphorylated S6 in the KRAS MUT but not WT DLD-1 line. (D) IL-22 protects against 5 fluorouracil mediated cell death in KRAS MUT but not KRAS WT DLD-1 cells as measured by MTT assay on DLD-1 KRAS MUT and WT cells pre-treated or not pre-treated for 48 h with 10 ng/mL IL-22, then treated with 50 μM 5-FU for 48 h. MTT was added 2 h prior to end of 48 h incubation. Formazan particles were dissolved in DMSO and absorbance was measured at 540 nm. Raw absorbances are blank corrected, normalized to a no treatment, and represented as a % viability compared to the no treatment condition. n=5 independent experiments with 3 experimental replicates. p values computed by non-parametric Mann-Whitney test comparing 5 FU alone to IL-22 pretreatment with 5 FU for each DLD-1 line.

### DESCRIPTION OF THE SEQUENCE LISTING

SEQ ID NO: 1 shows the amino acid sequence of human KRAS isoform a.

SEQ ID NO: 2 shows the amino acid sequence of human KRAS isoform b.

SEQ ID NO: 3 shows the amino acid sequence of the human IL-22RA1 protein.

SEQ ID NO: 4 shows the mRNA sequence of human IL22RA1.

SEQ ID NO: 5 shows the amino acid sequence of the human IL-22 protein.

SEQ ID NO: 6 shows the mRNA sequence of human IL22.

SEQ ID NO: 7 shows the amino acid sequence of the human IL-20 protein.

SEQ ID NO: 8 shows the mRNA sequence of human IL20.

SEQ ID NO: 9 shows the amino acid sequence of human IL-24 protein isoform 3.

SEQ ID NO: 10 shows the mRNA sequence of human IL-24 isoform 3.

SEQ ID NO: 11 shows the amino acid sequence of the human IL-22 neutralizing receptor IL-22-binding protein (IL-22BP or IL-22Rα2).

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “an inhibitor” includes two or more such inhibitors, or reference to “an oligonucleotide” includes two or more such oligonucleotide and the like.

All publications, patents and patent applications cited herein, whether supra or infra, are hereby incorporated by reference in their entirety.

### Method of Treating Colorectal Cancer

The method of the invention concerns treating colorectal cancer (also known as a colorectal tumour). Such cancers and tumours are known in the art. The colorectal cancer is preferably proximal colorectal cancer (or a proximal colorectal tumour). The proximal colon is the region of the large bowel upstream of the splenic flexure, meaning the caecum, the ascending colon and the transverse colon. Cancers or tumours in this region are also referred to as right-sided cancers or tumours. The invention may concern treating right-sided colorectal cancer or a right-sided colorectal tumour.

The colorectal cancer may be distal colorectal cancer (or a distal colorectal tumour). The distal colon is the region of the large bowel downstream of the splenic flexure, meaning the descending colon, the sigmoid colon and the rectum. Cancers or tumours in this region are also referred to as left-sided cancers or tumours. The invention may concern treating left-sided colorectal cancer or a left-sided colorectal tumour.

The cancer treated in accordance with the invention comprises a KRAS mutation and a high amount of IL-22 receptor. Before treatment in accordance with the invention, it is necessary to determine whether or not the cancer comprises a KRAS mutation and a high amount of IL-22 receptor. This can be done is several ways as discussed below. The presence of a KRAS mutation and a high amount of IL-22 receptor indicates that the cancer is suitable for treatment using an inhibitor of IL-22 signalling in accordance with the invention. The absence of a KRAS mutation and/or the absence of a high amount of IL-22 receptor indicates that the cancer is not suitable for treatment using an inhibitor of IL-22 signalling in accordance with the invention. The absence of a KRAS mutation and/or the presence of a low amount of IL-22 receptor indicates that the cancer is not suitable for treatment using an inhibitor of IL-22 signalling in accordance with the invention.

The method of the invention is preferably for treating colorectal cancer in a patient that has been selected for treatment on the basis that the cancer comprises a KRAS mutation and a high amount of IL-22 receptor. The method of the invention is preferably for treating colorectal cancer in a patient that has been selected for treatment on the basis that the cancer is proximal colorectal cancer which comprises a KRAS mutation and a high amount of IL-22 receptor. In preferred embodiments of the invention as discussed below, the method involves both selection and treatment.

### KRAS Mutations

The invention concerns the treatment of a cancer comprising a KRAS mutation. KRAS is a GTPase which hydrolyses GTP to GDP allowing for activation of a number of downstream signalling pathways including phosphatidyl-inositil and mitogen activated kinase pathways. Common mutations in KRAS reduce its intrinsic GTPase function, preventing hydrolysis of GTP to GDP, thus locking KRAS in its active state. This results in constitutive activation of downstream signalling pathways that can drive oncogenesis.

KRAS mutations are known in the art (see, for example, http://www.mycancergenome.org/content/disease/colorectal-cancer/kras/29/). A cancer comprises a KRAS mutation if one or more of the cells in the cancer comprise(s) a KRAS mutation. This can be tested as discussed below.

The cancer may comprise a mutation in the KRAS gene. The cancer may comprise a missense mutation. Missense mutations change the amino acid sequence of the KRAS protein and thus can reduce the function of the KRAS protein or abolish it altogether.

The cancer may comprise a nonsense mutation. This leads to decay of mRNA and thus a reduction in KRAS protein expression.

The cancer may comprise a frameshift mutation. The frameshift mutation may be a deletion frameshift mutation or an insertion frameshift mutation. Both types of mutation can decrease the function of the KRAS protein or abolish it altogether. Some frameshift mutations can also introduce a pre-mature stop codon and lead to loss of KRAS protein expression.

The cancer may comprise a deletion inframe mutation. This mutation may also decrease the function of the KRAS protein or abolish it altogether.

The mutations discussed above are preferably homozygous.

The KRAS cancer may lack the KRAS gene. In other words, the KRAS gene may be absent from the cancer.

Mutations in the KRAS gene may be identified using DNA sequencing including next-generation sequencing. This may also be done using Southern blotting, measuring copy-number variation and investigating KRAS promoter methylation.

In some instances, the mutation or absence of the KRAS gene may be due to a chromosome 12 abnormality, such as chromosome 12p deletion or rearrangement. The cancer may therefore comprise a chromosome abnormality, such as chromosome 12p deletion or rearrangement. Chromosome 3 abnormalities, such as chromosome 12p deletion or rearrangement, may be identified using cytogenetic analysis such as giemsa banding, fluorescence in situ hybridisation (FISH) or comparative genomic hybridization, such as array-comparative genomic hybridization (array CGH).

It will be clear from the above that mutations may affect the expression of the KRAS protein, its stability or its ability to function. The cancer may comprise a decreased amount of KRAS protein, such as a decreased amount of SEQ ID NO: 1 or 2 or a variant thereof as discussed in more detail below. The cancer may comprise a decreased amount of KRAS protein compared with normal cells of the same tissue type, i.e. colorectal cells, such as proximal or distal colorectal cells. The cancer may comprise a decreased amount of KRAS protein compared with cancers cells of the same tissue type, i.e. colorectal cancer cells, such as proximal or distal colorectal cancer cells, and without a KRAS mutation.

The amount of KRAS protein may be decreased by any amount. For instance, the amount of KRAS protein may be decreased by at least 10%, at least 30% at least 40%, at least 50%, at least 60%, at least 70%, at least 80%, at least 90% or at least 95% compared with the level of KRAS in normal cells of the same type or cancers cells of the same tissue type and without a KRAS mutation. The amount of KRAS protein can be measured using known techniques. The amount of KRAS protein can be measured using immunohistochemistry, western blotting, mass spectrometry or fluorescence-activated cell sorting (FACS). Suitable antibodies against KRAS are available. For example, such antibodies are available from Abeam®.

The cancer may comprise a KRAS protein with decreased function. The cancer may comprise a KRAS protein with decreased function compared with normal (i.e. wild-type or native) KRAS protein, such as SEQ ID NO: 1 or 2 or a variant thereof. The function of the KRAS protein may be decreased by any amount and in particular the % amounts discussed above in relation of KRAS amount. The cancer may comprise KRAS protein with no function (i.e. a lack of function or an abolished function). The function of KRAS protein, for instance its ability to hydrolyse GTP, can be assayed as using known techniques. The cancer may comprise no KRAS protein (i.e. may lack KRAS protein).

It will be clear from the above that mutations may affect the amount of the KRAS mRNA. The cancer may comprise a decreased amount of KRAS mRNA. The cancer may comprise a decreased amount of KRAS mRNA compared with normal cells of the same tissue type or cancers cells of the same tissue type and without a KRAS mutation. The amount of the KRAS mRNA may be decreased by any amount and in particular the % amounts discussed above in relation of KRAS protein. The amount of KRAS mRNA can be measured using quantitative reverse transcription polymerase chain reaction (qRT-PCR), such as real time qRT-PCR, northern blotting or microarrays. Mutations in KRAS mRNA may be identified using RNA sequencing including next-generation sequencing. KRAS mRNA preferably has a sequence which encodes one of the sequences shown in SEQ ID NO: 1 or 2 or a variant thereof as discussed in more detail below.

Human KRAS protein is typically present in two isoforms as shown in SEQ ID NOs: 1 and 2. The cancer preferably comprises (i) a variant of one of these sequences comprising one or more point mutations or (ii) a polynucleotide which encodes the variant in (i). The cancer preferably comprises (i) a variant of the sequence shown in SEQ ID NO: 1 or 2 which comprises one or more point mutations or (ii) a polynucleotide which encodes the variant in (i). Polynucleotides are defined in more detail below. The polynucleotide which encodes the variant of SEQ ID NO: 1 or 2 in the cancer is typically DNA or RNA, such as mRNA.

The variant of SEQ ID NO: 1 or 2 preferably comprises a point mutation at one or more of positions 12, 13, 14, 59, 61, 117, 120, 144, 145 and 146 of SEQ ID NO: 1 or 2. The variant may comprise a point mutation at any number and combination of these positions.

The variant of SEQ ID NO: 1 or 2 preferably comprises one or more of the following point mutations (a) G12A, G12C, G12D, G12R, G12S or G12V, (b) G13A, G13C, G13D, G13R or G13V, (c) V14I, (d) A59G, (e) Q61H, Q61K Q61L or Q61R, (f) K117N, (g) L120V, (h) S145T and (i) A146P, A146T and A146V. The variant may comprise any number and combination of (a) to (i). The variant most preferably comprises one of (a) to (i).

Over the entire length of the amino acid sequence of SEQ ID NO: 1 or 2, the variant will preferably be at least 90% homologous to that sequence based on amino acid identity, i.e. have at least 90% amino acid identity over the entire sequence. More preferably, the variant may be at least 95%, 97% or 99% homologous based on amino acid identity (or identical) to the amino acid sequence of SEQ ID NO: 1 or 2 over the entire sequence. The variant preferably only comprises the one or more point mutations.

Standard methods in the art may be used to determine homology. For example the UWGCG Package provides the BESTFIT program, which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/).

The presence of one or more point mutations may be identified using any known method. The presence of point mutations are typically typically identified using the polynucleotide, such as DNA or mRNA, encoding the KRAS protein the cancer cells. Sequencing or identifying the polynucleotides allows the presence or absence of the one or more point mutations to be determined. The presence of one or more point mutations may be measured by DNA or RNA sequencing including next-generation sequencing. The presence of one or more point mutations may also be measured by denaturing gradient gel electrophoresis (DGGE), temperature gradient gel electrophoresis (TGGE), single-strand confirmation polymorphism (SSCP), heteroduplex analysis (HET), RNAasse A cleavage method, chemical cleavage method (CCM), enzyme mismatch cleavage (EMC), cleavage fragment length polymorphism (CFLP), mutation detection by mismatch inding proteins, protein truncation test (PTT), allele-specific oligonucleotide (ASO) DNA hybridization of DNA chips, naturally-occurring or-primer-mediated restriction fragment analysis, allele-specific amplification (ASA) or oligonucleotide ligation assay (OLA).

The KRAS mutation is typically measured in a cancer biopsy obtained from the patient. The biopsy tissue may be formalin fixed paraffin embedded (FFPE) tissue or fresh tissue. Any of the methods discussed above may be carried out on the cancer biopsy. Such methods may also be carried out on cancer cells circulating in the blood of the patient. The RNA methods may be carried out on urinary or blood exosomes. The DNA methods may be carried out on circulating free DNA in blood. The methods may also be carried out on a stool sample.

### IL-22 Receptor

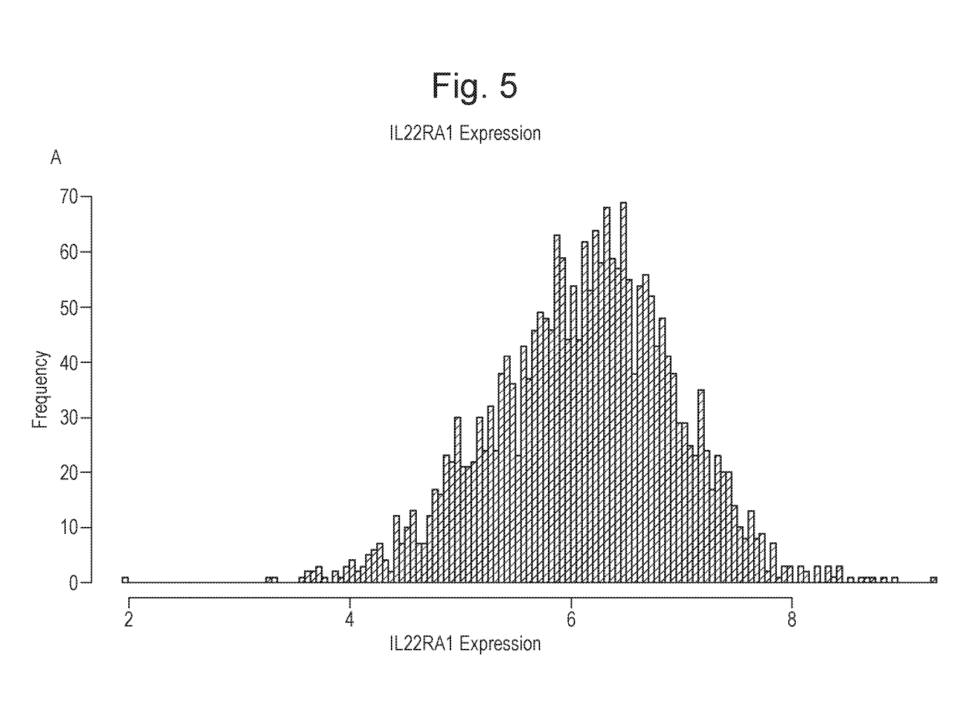

The cancer comprises a high amount of interleukin 22 (IL-22) receptor. The cancer typically comprises a high amount of IL-22 receptor relative to other cancers of the same type, i.e. other colorectal cancers. Proximal colorectal cancer typically comprises a high amount of IL-22 receptor relative to other cancers of the same type, i.e. other proximal colorectal cancers. As can be seen from FIG. 5, the expression of IL-22 receptor in proximal colorectal cancers is approximately a normal distribution. Distal colorectal cancer typically comprises a high amount of IL-22 receptor relative to other cancers of the same type, i.e. other distal colorectal cancers.

The cancer preferably comprises an amount of IL-22 receptor which is greater than the 60^{th} or 67th percentile of amount in a cohort of colorectal cancers, such as a cohort of proximal colorectal cancers or a cohort of distal colorectal cancers. The cancer may comprise an amount of IL-22 receptor which is greater than the 60^{th}, 61^{st}, 62^{nd}, 63^{rd}, 64^{th}, 65^{th}, 66^{th}, 67^{th}, 68^{th}, 69^{th}, 70^{th}, 71^{st}, 72^{nd}, 73^{rd}, 74^{th}, 75^{th}, 76^{th}, 77^{th}, 78^{th}, 79^{th}, 80^{th}, 81^{st}, 82^{nd}, 83^{rd}, 84^{th}, 85^{th}, 86^{th}, 87^{th}, 88^{th}, 89^{th}, 90^{th}, 91^{st}, 92^{nd}, 93^{rd}, 94^{th}, 95^{th}, 96^{th}, 97^{th}, 98^{th} or 99^{th} percentile of amount in a cohort of colorectal cancers, such as a cohort of proximal colorectal cancers or a cohort of distal colorectal cancers. The cohort typically comprises at least 10 colorectal cancers, such as at least 20, at least 30, at least 50 or at least 100 colorectal cancers. The percentile of amount can be determined using standard statistical techniques.

On an individual case basis, a cancer can be tested for high levels of IL-22 receptor if the mRNA expression of the IL-22 receptor gene, such as IL-22RA1, as measured by a relevant technique, such as quantitative real-time PCR, is measured as a ratio of the average expression of one or more reference (or control) genes. For example, using RNA-Seq data from a 203-case colorectal cancer cohort derived from The Cancer Genome Atlas project, the 67th percentile of IL-22 receptor expression, when normalized to the average expression of GPX1, VDAC2, PGK1, ATP5E, and UBB (these are used as reference genes in the Oncotype Dx colorectal cancer test (http://colon-cancer.oncotypedx.com), yields a value of approximately 0.05. Thus, when determined in this fashion, a patient with a normalized IL-22 receptor value >0.05 would be considered IL-22 receptor-high.

The cancer preferably comprises an amount of IL-22 receptor which is greater than the ratio of the 60^{th} or 67^{th} percentile (or any of the percentiles listed above) of amount in a cohort of colorectal cancers to the amount of one or more reference (or control) genes. The one or more reference (or control) genes are preferably GPX1, VDAC2, PGK1, ATP5E, and UBB. The amounts of the different genes are preferably measured using the same technique.

The IL-22 receptor is a heterodimeric receptor comprised of the IL-22 receptor alpha 1 (IL-22RA1) subunit and an IL-10 receptor 2 (IL-10RB2) subunit, which is also utilized by several other members of the IL-10 family.^{8,9} The cancer preferably comprises a high amount of IL-22 receptor subunit alpha-1 (IL-22RA1). The cancer preferably comprises an amount of IL-22RA1 which is greater than the 60^{th} or 67^{th} percentile of amount in a cohort of colorectal cancers, such as a cohort of proximal colorectal cancers or a cohort of distal colorectal cancers. The amount may be greater than any of the percentiles of amount discussed above and/or the cohort can have any number of cancers as discussed above.

The cancer preferably comprises a high amount of IL-22RA1 protein and/or a high amount of IL22RA1 mRNA. The cancer may comprises a high amount of IL-22RA1 protein, such as an amount of IL-22RA1 protein which is greater than the 60^{th} or 67^{th} percentile of amount in a cohort of colorectal cancers, such as a cohort of proximal colorectal cancers or a cohort of distal colorectal cancers. The amount may be greater than any of the percentiles of amount discussed above and/or the cohort can have any number of cancers as discussed above.

The cancer preferably comprises an amount of IL-22RA1 protein which is greater than the ratio of the 60^{th} or 67^{th} percentile (or any of the percentiles listed above) of amount in a cohort of colorectal cancers to the amount of one or more reference (or control) proteins. The one or more reference (or control) proteins are preferably GPX1, VDAC2, PGK1, ATP5E, and UBB. The amounts of the different proteins are preferably measured using the same technique.

The amount of IL-22RA1 protein can be measured using known techniques. The amount of IL-22RA1 protein can be measured using immunohistochemistry, western blotting, mass spectrometry or fluorescence-activated cell sorting (FACS). Suitable antibodies against IL-22RA1 protein are available, for example from Human Protein Atlas.

The cancer may comprise a high amount of IL22RA1 mRNA, such as an amount of IL-22RA1 mRNA which is greater than the 60^{th} or 67^{th} percentile of amount in a cohort of colorectal cancers, such as a cohort of proximal colorectal cancers or a cohort of distal colorectal cancers. The amount may be greater than any of the percentiles of amount discussed above and/or the cohort can have any number of cancers as discussed above.

The cancer preferably comprises an amount of IL-22RA1 mRNA which is greater than the ratio of the 60^{th} or 67^{th} percentile (or any of the percentiles listed above) of amount in a cohort of colorectal cancers to the amount of one or more reference (or control) mRNAs. The one or more reference (or control) mRNAs are preferably GPX1, VDAC2, PGK1, ATP5E, and UBB. The amounts of the different mRNAs are preferably measured using the same technique.

The amount of IL-22RA1 mRNA can be measured using quantitative reverse transcription polymerase chain reaction (qRT-PCR), such as real time qRT-PCR, northern blotting or microarrays.

The IL-22RA1 protein preferably comprises the sequence shown in SEQ ID NO: 3 or a naturally-occurring variant thereof. The naturally-occurring variant has the ability to form a functional IL-22 receptor, i.e. bind IL-22, form a heterodimer and activate signal transduction pathways. This can be determined using routine IL-22 signalling assays. For instance, cells in vitro/ex vivo may be contacted with the variant for 24 h followed by qPCR based detection of target genes transcription (ie. SOCS3, OFLM4) and Western Blot based detection of phosphorylation events in IL-22 signalling cascade (ie. phosphorylated STAT3). The naturally-zoccurring variant is typically a polymorphism. Over the entire length of the amino acid sequence of SEQ ID NO: 3, a naturally-occurring variant will preferably be at least 90% homologous to that sequence based on amino acid identity, i.e. have at least 90% amino acid identity over the entire sequence. More preferably, the naturally-occurring variant may be at least 95%, 97% or 99% homologous based on amino acid identity (or identical) to the amino acid sequence of SEQ ID NO: 3 over the entire sequence. Homology may be measured as discussed above.

The IL-22RA1 mRNA preferably comprises the sequence shown in SEQ ID NO: 4 or a naturally-occurring variant thereof. The naturally-occurring variant encodes a protein which has the ability to form a functional IL-22 receptor. The naturally-occurring variant is typically a polymorphism. Over the entire length of the sequence of SEQ ID NO: 4, a naturally-occurring variant will preferably be at least 90% homologous to that sequence based on nucleotide identity over the entire sequence, i.e. have at least 90% nucleotide identity over the entire sequence. More preferably, the naturally-occurring variant may be at least 95%, 97% or 99% homologous based on nucleotide identity (or identical) to the nucleotide sequence of SEQ ID NO: 4 over the entire sequence. Homology may be measured as discussed above

The amount of IL-22 receptor is typically measured in a cancer biopsy obtained from the patient. The cancer biopsy may be the same as or different from the biopsy used for the KRAS mutation analysis. Any of the methods discussed above may be carried out on a cancer biopsy. Such methods may also be carried out on cancer cells circulating in the blood of the patient. The RNA methods may be carried out on urinary or blood exosomes. The DNA methods may be carried out on circulating free DNA in blood. The methods may also be carried out on a stool sample.

### Patient

Any patient may be treated in accordance with the invention. The patient is typically human. However, patient may be another mammalian animal, such as a commercially farmed animal, such as a horse, a cow, a sheep, a fish, a chicken or a pig, a laboratory animal, such as a mouse or a rat, or a pet, such as a guinea pig, a hamster, a rabbit, a cat or a dog.

### Inhibitors

An inhibitor of IL-22 signalling is any molecule that decreases or reduces 11-22 signalling. The inhibitor may decrease IL-22 signalling by any amount. For instance, the signalling may be decreased by at least 10%, at least 30% at least 40%, at least 50%, at least 60%, at least 70%, at least 80%, at least 90% or at least 95%. An inhibitor may abolish IL-22 signalling (i.e. the function is decreased by 100%). IL-22 signalling may be measured using known techniques. The extent to which an inhibitor affects IL-22 may be determined by measuring the signalling in cells in the presence and absence of the inhibitor. The cells may be normal cells or may be cancer cells. The cells are typically colorectal cells, such as proximal colorectal cells or distal colorectal cells. The cells are more typically colorectal cancer cells, such as proximal colorectal cancer cells or distal colorectal cancer cells. The activity of the inhibitor may be measured by determining the effect of the inhibitor on the ability of a ligand of the IL-22 receptor to activate any of the IL-22 receptor signal transduction pathways discussed above.

The inhibitor may affect the IL-22 signalling in any manner. For instance, the inhibitor may decrease the amount of the IL-22 receptor, for instance by decreasing the expression of or increasing the degradation of the IL-22 receptor. The inhibitor may decrease the activity of the IL-22 receptor, for instance by binding to the IL-22 receptor or the molecule(s) which the IL-22 receptor activates. The inhibitor may decrease the amount of and/or the activity of an IL-22 receptor ligand, such as IL-22, interleukin 20 (IL-20) or interleukin (IL-24).

The inhibitor may be a competitive inhibitor (which binds the active site of the molecule to which it binds) or an allosteric inhibitor (which does not bind the active site of the molecule to which it binds). The inhibitor may be reversible. The inhibitor may be irreversible.

### Inhibitors of the IL-22 Receptor or IL-22RA1

The inhibitor is preferably an inhibitor of the IL-22 receptor or IL-22RA1. The inhibitor may decrease the production of or expression of the IL-22 receptor or IL-22RA1. The inhibitor may decrease the transcription of the IL-22 receptor or IL-22RA1. The inhibitor may disrupt the DNA of the IL-22 receptor or IL-22RA1, for instance by site-specific mutagenesis using methods such as Zinc-finger nucleases. The inhibitor may decrease the mRNA level of the IL-22 receptor or IL-22RA1 or interfere with the processing of the IL-22 receptor or IL-22RA1 mRNA, for instance by antisense RNA or RNA interference. This is discussed in more detail below.

The inhibitor may increase protein degradation of the IL-22 receptor or IL-22RA1. The inhibitor may increase the level of natural inhibitors of the IL-22 receptor or IL-22RA1. The inhibitor may decrease the function of the IL-22 receptor or IL-22RA1 by inhibitory phosphorylation, ubiquitylation, sumoylation or the like.

The inhibitor of the IL-22 receptor or IL-22RA1 is preferably a small molecule inhibitor, a protein, an antibody, a polynucleotide, an oligonucleotide, an antisense RNA, small interfering RNA (siRNA) or small hairpin RNA (shRNA).

The inhibitor of the IL-22 receptor or IL-22RA1 may be a protein. The inhibitor is preferably a reduced-function form of the IL-22 receptor or IL-22RA1. The function of the reduced-function form may be reduced/decreased by any amount. For instance, the function may be reduced/decreased by at least 10%, at least 30% at least 40%, at least 50%, at least 60%, at least 70%, at least 80%, at least 90% or at least 95% compared with wild-type IL-22 receptor or IL-22RA1. The inhibitor may be a non-functional form of the IL-22 receptor or IL-22RA1. A reduced-function or non-functional form of the IL-22 receptor or IL-22RA1 will compete with native (i.e. wild-type) the IL-22 receptor or IL-22RA1 and reduce IL-22 signalling.

The amino acid sequence of human IL-22RA1 is shown in SEQ ID NO: 3. The inhibitor is preferably a reduced-function variant or non-functional variant of SEQ ID NO: 3. A reduced-function variant is a protein that has an amino acid sequence which varies from that of SEQ ID NO: 3 and has a reduced/decreased the ability to form a functional IL-22 receptor or activate signal transduction pathways. The function may be reduced/decreased by any amount as discussed above. A non-functional variant is a protein that has an amino acid sequence which varies from that of SEQ ID NO: 3 and does not have the ability to form a functional IL-22 receptor or activate signal transduction pathways. For instance, the non-functional variant may have one or more mutations in the site that forms the heterodimeric receptor or interacts with the signal transduction pathways. The non-functional variant may also be a truncated form that sequesters IL-22 or other IL-22 receptor ligands. This is discussed in more detail below. The ability of a variant to function as IL-22 receptor can be assayed using any method known in the art. Suitable methods are described above. The comparative functional ability of reduced-function and non-functional variants is typically measured in comparison to the wild-type IL-22 receptor.

Over the entire length of the amino acid sequence of SEQ ID NO: 3, a reduced-function or non-functional variant will preferably be at least 50% homologous to that sequence based on amino acid identity, i.e. have at least 50% amino acid identity over the entire sequence. More preferably, the reduced-function or non-functional variant may be at least 60%, at least 70%, at least 80%, at least 85%, at least 90% and more preferably at least 95%, 97% or 99% homologous based on amino acid identity (or identical) to the amino acid sequence of SEQ ID NO: 3 over the entire sequence. There may be at least 80%, for example at least 85%, 90% or 95%, amino acid identity over a stretch of 100 or more, for example 200 or 300 or more, contiguous amino acids (“hard homology”).

Amino acid substitutions may be made to the amino acid sequence of SEQ ID NO: 3, for example up to 1, 2, 3, 4, 5, 10, 20, 30, 50, 100 or 200 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in the Table below. Where amino acids have similar polarity, this can also be determined by reference to the hydropathy scale for amino acid side chains in the second table below.

TABLE
Chemical properties of amino acids

| | |
|---|---|
| Ala | aliphatic, hydrophobic, neutral |
| Cys | polar, hydrophobic, neutral |
| Asp | polar, hydrophilic, charged (−) |
| Glu | polar, hydrophilic, charged (−) |
| Phe | aromatic, hydrophobic, neutral |
| Gly | aliphatic, neutral |
| His | aromatic, polar, hydrophilic, charged (+) |
| Ile | aliphatic, hydrophobic, neutral |
| Lys | polar, hydrophilic, charged (+) |
| Leu | aliphatic, hydrophobic, neutral |
| Met | hydrophobic, neutral |
| Asn | polar, hydrophilic, neutral |
| Pro | hydrophobic, neutral |
| Gln | polar, hydrophilic, neutral |
| Arg | polar, hydrophilic, charged (+) |
| Ser | polar, hydrophilic, neutral |
| Thr | polar, hydrophilic, neutral |
| Val | aliphatic, hydrophobic, neutral |
| Trp | aromatic, hydrophobic, neutral |
| Tyr | aromatic, polar, hydrophobic |

TABLE
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One or more amino acid residues of the amino acid sequence of SEQ ID NO: 3 may additionally be deleted from the polypeptides described above. Up to 1, 2, 3, 4, 5, 10, 20, 30 or 50 residues may be deleted, or more.

Reduced-function or non-functional variants may include fragments of SEQ ID NO: 3. Such fragments typically retain the domain of SEQ ID NO: 3 which binds IL-22 or other ligands of the IL-22 receptor but are reduced-function or non-functional. Fragments may be at least 200, 300, 400 or 500 amino acids in length. One or more amino acids may be alternatively or additionally added to the polypeptides described above.

A preferred non-functional variant of IL-22RA1 is shown in SEQ ID NO: 11. This is the human IL-22-binding protein (IL-22BP or IL-22Rα2), a soluble receptor produced by CD11c⁺ cells that sequesters IL-22 and prevents its activity. The inhibitor preferably comprises the sequence shown in SEQ ID NO: 11 or a variant thereof. The variant has the ability to bind IL-22 or another ligand of the IL-22 receptor, such as interleukin 20 (IL-20) or interleukin (IL-24). This can be tested using standard binding assays. Over the entire length of the amino acid sequence of SEQ ID NO: 11, a variant will preferably be at least 80% homologous to that sequence based on amino acid identity, i.e. have at least 90% amino acid identity over the entire sequence. More preferably, the variant may be at least 90%, at least 95%, 97% or 99% homologous based on amino acid identity (or identical) to the amino acid sequence of SEQ ID NO: 11 over the entire sequence. Homology cam be measured as discussed above. The variant may include any of the modifications and substitutions discussed above with reference to the other non-functional variants.

Other preferred non-functional variants of IL-22RA1 are described in US 20120207761 A1 and US 20080242839 A1.

Alternatively, the inhibitor may be a polynucleotide encoding a reduced-function or non-functional variant of the IL-22 receptor or IL-22RA1. The reduced-function or non-functional variant may be any of those discussed above.

A polynucleotide, such as a nucleic acid, is a polymer comprising two or more nucleotides. The nucleotides can be naturally occurring or artificial. A nucleotide typically contains a nucleobase, a sugar and at least one linking group, such as a phosphate, 2′O-methyl, 2′ methoxy-ethyl, phosphoramidate, methylphosphonate or phosphorothioate group. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate. The nucleotides are preferably selected from AMP, TMP, GMP, UMP, dAMP, dTMP, dGMP or dCMP.

The nucleotides may contain additional modifications. In particular, suitable modified nucleotides include, but are not limited to, 2′amino pyrimidines (such as 2′-amino cytidine and 2′-amino uridine), 2′-hyrdroxyl purines (such as, 2′-fluoro pyrimidines (such as 2′-fluorocytidine and 2′fluoro uridine), hydroxyl pyrimidines (such as 5′-α-P-borano uridine), 2′-O-methyl nucleotides (such as 2′-O-methyl adenosine, 2′-O-methyl guanosine, 2′-O-methyl cytidine and 2′-O-methyl uridine), 4′-thio pyrimidines (such as 4′-thio uridine and 4′-thio cytidine) and nucleotides have modifications of the nucleobase (such as 5-pentynyl-2′-deoxy uridine, 5-(3-aminopropyl)-uridine and 1,6-diaminohexyl-N-5-carbamoylmethyl uridine).

One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light.

The nucleotides in the polynucleotide may be attached to each other in any manner. The nucleotides may be linked by phosphate, 2′O-methyl, 2′ methoxy-ethyl, phosphoramidate, methylphosphonate or phosphorothioate linkages. The nucleotides are typically attached by their sugar and phosphate groups as in nucleic acids. The nucleotides may be connected via their nucleobases as in pyrimidine dimers.

The polynucleotide can be a nucleic acid, such as deoxyribonucleic acid (DNA) or a ribonucleic acid (RNA). The polynucleotide may be any synthetic nucleic acid known in the art, such as peptide nucleic acid (PNA), glycerol nucleic acid (GNA), threose nucleic acid (TNA), locked nucleic acid (LNA), morpholino nucleic acid or other synthetic polymers with nucleotide side chains. The polynucleotide may be single stranded or double stranded.

The polynucleotide sequence preferably encodes a reduced-function or non-functional variant of SEQ ID NO: 3 as discussed above. The polynucleotide sequence preferably comprises a variant of SEQ ID NO: 4 with at least 50%, at least 60%, at least 70%, at least 80%, at least 90%, at least 95%, at least 97%, at least 98% or at least 99% homology based on nucleotide identity over the entire sequence, i.e. nucleotide identity over the entire sequences. There may be at least 80%, for example at least 85%, 90% or 95% nucleotide identity over a stretch of 300 or more, for example 400, 500, 600, 700, 800 or 900 or more, contiguous nucleotides (“hard homology”). Homology may be calculated as described above.

The polynucleotide sequence preferably comprises a sequence which encodes SEQ ID NO: 11 or any its variants discussed above.

Polynucleotide sequences may be derived and replicated using standard methods in the art, for example using PCR involving specific primers. It is straightforward to generate polynucleotide sequences using such standard techniques.

The amplified sequences may be incorporated into a recombinant replicable vector such as a cloning vector. The vector may be used to replicate the polynucleotide in a compatible host cell. Thus polynucleotide sequences may be made by introducing the polynucleotide into a replicable vector, introducing the vector into a compatible host cell, and growing the host cell under conditions which bring about replication of the vector. The vector may be recovered from the host cell. Suitable host cells for cloning of polynucleotides are known in the art and described in more detail below.

The polynucleotide sequence may be cloned into any suitable expression vector. In an expression vector, the polynucleotide sequence encoding a construct is typically operably linked to a control sequence which is capable of providing for the expression of the coding sequence by the host cell. Such expression vectors can be used to express a construct.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the vector.

The expression vector may then be introduced into a suitable host cell. Thus, a construct can be produced by inserting a polynucleotide sequence encoding a construct into an expression vector, introducing the vector into a compatible bacterial host cell, and growing the host cell under conditions which bring about expression of the polynucleotide sequence. The vectors may be for example, plasmid, virus or phage vectors provided with an origin of replication, optionally a promoter for the expression of the said polynucleotide sequence and optionally a regulator of the promoter. The vectors may contain one or more selectable marker genes, for example an ampicillin resistance gene. Promoters and other expression regulation signals may be selected to be compatible with the host cell for which the expression vector is designed. A T7, trc, lac, ara or λ_{L} promoter is typically used.

The host cell typically expresses the construct at a high level. Host cells transformed with a polynucleotide sequence encoding a construct will be chosen to be compatible with the expression vector used to transform the cell. The host cell is typically bacterial and preferably E. coli. Any cell with a λ DE3 lysogen, for example C41 (DE3), BL21 (DE3), JM109 (DE3), B834 (DE3), TUNER, Origami and Origami B, can express a vector comprising the T7 promoter. Inhibitors of the IL-22 receptor or IL-22RA1 may also reduce amounts of the IL-22 receptor or IL-22RA1 present in the patient or the cancer, for example by knocking down expression of the IL-22 receptor or IL-22RA1. Antisense and RNA interference (RNAi) technology for knocking down protein expression are well known in the art and standard methods can be employed to knock down expression of the IL-22 receptor or IL-22RA1.

Both antisense and siRNA technology interfere with mRNA. Antisense oligonucleotides interfere with mRNA by binding to (hybridising with) a section of the mRNA. The antisense oligonucleotide is therefore designed to be complementary to the mRNA (although the oligonucleotide does not have to be 100% complementary as discussed below). In other words, the antisense oligonucleotide may be a section of the cDNA. Again, the oligonucleotide sequence may not be 100% identical to the cDNA sequence. This is also discussed below.

RNAi involves the use of double-stranded RNA, such small interfering RNA (siRNA) or small hairpin RNA (shRNA), which can bind to the mRNA and inhibit protein expression.

Accordingly, the inhibitor preferably comprises an oligonucleotide which specifically hybridises to a part of the IL-22 receptor mRNA or the IL-22RA1 mRNA. The inhibitor preferably comprises an oligonucleotide which specifically hybridises to a part of SEQ ID NO: 4 (human IL-22RA1 mRNA) or any naturally-occurring variant thereof as discussed above. Oligonucleotides are short nucleotide polymers which typically have 50 or fewer nucleotides, such 40 or fewer, 30 or fewer, 22 or fewer, 21 or fewer, 20 or fewer, 10 or fewer or 5 or fewer nucleotides. The oligonucleotide used in the invention is preferably 20 to 25 nucleotides in length, more preferably 21 or 22 nucleotides in length. The nucleotides can be naturally occurring or artificial. The nucleotides can be any of those described above.

An oligonucleotide preferably specifically hybridises to a part of SEQ ID NO: 4 or any naturally-occurring variant thereof as discussed above, hereafter called the target sequence. The length of the target sequence typically corresponds to the length of the oligonucleotide. For instance, a 21 or 22 nucleotide oligonucleotide typically specifically hybridises to a 21 or 22 nucleotide target sequence. The target sequence may therefore be any of the lengths discussed above with reference to the length of the oligonucleotide. The target sequence is typically consecutive nucleotides within the target polynucleotide.

An oligonucleotide “specifically hybridises” to a target sequence when it hybridises with preferential or high affinity to the target sequence but does not substantially hybridise, does not hybridise or hybridises with only low affinity to other sequences.

An oligonucleotide “specifically hybridises” if it hybridises to the target sequence with a melting temperature (Tₘ) that is at least 2° C., such as at least 3° C., at least 4° C., at least 5° C., at least 6° C., at least 7° C., at least 8° C., at least 9° C. or at least 10° C., greater than its Tₘ for other sequences. More preferably, the oligonucleotide hybridises to the target sequence with a Tₘ that is at least 2° C., such as at least 3° C., at least 4° C., at least 5° C., at least 6° C., at least 7° C., at least 8° C., at least 9° C., at least 10° C., at least 20° C., at least 30° C. or at least 40° C., greater than its Tₘ for other nucleic acids. Preferably, the portion hybridises to the target sequence with a Tₘ that is at least 2° C., such as at least 3° C., at least 4° C., at least 5° C., at least 6° C., at least 7° C., at least 8° C., at least 9° C., at least 10° C., at least 20° C., at least 30° C. or at least 40° C., greater than its Tₘ for a sequence which differs from the target sequence by one or more nucleotides, such as by 1, 2, 3, 4 or 5 or more nucleotides. The portion typically hybridises to the target sequence with a Tₘ of at least 90° C., such as at least 92° C. or at least 95° C. Tₘ can be measured experimentally using known techniques, including the use of DNA microarrays, or can be calculated using publicly available Tₘ calculators, such as those available over the internet.

Conditions that permit the hybridisation are well-known in the art (for example, Sambrook et al., 2001, Molecular Cloning: a laboratory manual, 3rd edition, Cold Spring Harbour Laboratory Press; and Current Protocols in Molecular Biology, Chapter 2, Ausubel et al., Eds., Greene Publishing and Wiley-Interscience, New York (1995)). Hybridisation can be carried out under low stringency conditions, for example in the presence of a buffered solution of 30 to 35% formamide, 1 M NaCl and 1% SDS (sodium dodecyl sulfate) at 37° C. followed by a 20 wash in from 1× (0.1650 M Na⁺) to 2× (0.33 M Na⁺) SSC (standard sodium citrate) at 50° C. Hybridisation can be carried out under moderate stringency conditions, for example in the presence of a buffer solution of 40 to 45% formamide, 1 M NaCl, and 1% SDS at 37° C., followed by a wash in from 0.5× (0.0825 M Na⁺) to 1× (0.1650 M Na⁺) SSC at 55° C. Hybridisation can be carried out under high stringency conditions, for example in the presence of a buffered solution of 50% formamide, 1 M NaCl, 1% SDS at 37° C., followed by a wash in 0.1× (0.0165 M Na⁺) SSC at 60° C.

The oligonucleotide may comprise a sequence which is substantially complementary to the target sequence. Typically, the oligonucleotides are 100% complementary. However, lower levels of complementarity may also be acceptable, such as 95%, 90%, 85% and even 80%. Complementarity below 100% is acceptable as long as the oligonucleotides specifically hybridise to the target sequence. An oligonucleotide may therefore have 1, 2, 3, 4, 5, 6, 7, 8, 9, 10 or more mismatches across a region of 5, 10, 15, 20, 21, 22, 30, 40 or 50 nucleotides.

Alternatively, the inhibitor preferably comprises an oligonucleotide which comprises 50 or fewer consecutive nucleotides from the reverse complement of (a) SEQ ID NO: 4 or (b) or any naturally-occurring variant thereof as discussed above. The oligonucleotide may be any of the lengths discussed above. It is preferably 21 or 22 nucleotides in length. The oligonucleotide may comprise any of the nucleotides discussed above, including the modified nucleotides.

The oligonucleotide can be a nucleic acid, such as any of those discussed above. The oligonucleotide is preferably RNA.

The oligonucleotide may be single stranded. The oligonucleotide may be double stranded. The oligonucleotide may comprise a hairpin.

Oligonucleotides may be synthesised using standard techniques known in the art. Alternatively, oligonucleotides may be purchased. Suitable sources are shown in Table 6.

The inhibitor is preferably an antibody which specifically binds the IL-22 receptor or IL-22RA1. The antibody preferably binds the sequence shown in SEQ ID NO: 3 or a naturally-occurring variant as discussed above.

An antibody “specifically binds” to a protein when it binds with preferential or high affinity to that protein but does not substantially bind, does not bind or binds with only low affinity to other proteins. For instance, an antibody “specifically binds” to SEQ ID NO: 3 or a naturally-occurring variant when it binds with preferential or high affinity to SEQ ID NO: 3 or a naturally-occurring variant but does not substantially bind, does not bind or binds with only low affinity to other human proteins.

An antibody binds with preferential or high affinity if it binds with a Kd of 1×10-7 M or less, more preferably 5×10-8 M or less, more preferably 1×10-8 M or less or more preferably 5×10-9 M or less. An antibody binds with low affinity if it binds with a Kd of 1×10-6 M or more, more preferably 1×10-5 M or more, more preferably 1×10-4 M or more, more preferably 1×10-3 M or more, even more preferably 1×10-2 M or more. A variety of protocols for competitive binding or immunoradiometric assays to determine the specific binding capability of compounds, such as antibodies or antibody constructs and oligonucleotides are well known in the art (see for example Maddox et al, J. Exp. Med. 158, 1211-1226, 1993).

The antibody may be, for example, a monoclonal antibody, a polyclonal antibody, a single chain antibody, a chimeric antibody, a bispecific antibody, a CDR-grafted antibody or a humanized antibody. The antibody may be an intact immunoglobulin molecule or a fragment thereof such as a Fab, F(ab′)2 or Fv fragment.

A preferred antibody is disclosed in U.S. Pat. No. 7,537,761.

### Inhibitors of Ligands of the IL-22 Receptor

The inhibitor is preferably an inhibitor of a ligand of the IL-22 receptor. The inhibitor is preferably an inhibitor of IL-22 (IL-22), interleukin 20 (IL-20) or interleukin (IL-24). The inhibitor may decrease the production of or expression of IL-22, IL-20 or IL-24. The inhibitor may decrease the transcription of IL-22, IL-20 or IL-24. The inhibitor may disrupt the DNA of IL-22, IL-20 or IL-24, for instance by site-specific mutagenesis using methods such as Zinc-finger nucleases. The inhibitor may decrease the mRNA level of IL-22, IL-20 or IL-24 or interfere with the processing of IL-22, IL-20 or IL-24 mRNA, for instance by antisense RNA or RNA interference. This is discussed in more detail below.

The inhibitor may increase protein degradation of IL-22, IL-20 or IL-24. The inhibitor may increase the level of natural inhibitors of IL-22, IL-20 or IL-24. The inhibitor may decrease the function of IL-22, IL-20 or IL-24 by inhibitory phosphorylation, ubiquitylation, sumoylation or the like.

The inhibitor of IL-22, IL-20 or IL-24 is preferably a small molecule inhibitor, a protein, an antibody, a polynucleotide, an oligonucleotide, an antisense RNA, small interfering RNA (siRNA) or small hairpin RNA (shRNA).

The inhibitor of IL-22, IL-20 or IL-24 may be a protein. The inhibitor is preferably a reduced-function form of IL-22, IL-20 or IL-24. The function of the reduced-function form may be reduced/decreased by any amount. For instance, the function may be reduced/decreased by at least 10%, at least 30% at least 40%, at least 50%, at least 60%, at least 70%, at least 80%, at least 90% or at least 95% compared with wild-type IL-22, IL-20 or IL-24. The inhibitor may be a non-functional form of IL-22, IL_20 or IL-24. A reduced-function or non-functional form of IL-22, IL-20 or IL-24 will compete with native (i.e. wild-type) IL-22, IL-20 or IL-24 and reduce IL-22 signalling.

The amino acid sequence of human IL-22 is shown in SEQ ID NO: 5. The amino acid sequence of human IL-20 is shown in SEQ ID NO: 7. The amino acid sequence of human IL-24 isoform 3 is shown in SEQ ID NO: 9. The inhibitor is preferably a reduced-function variant of, such as a non-functional variant of, SEQ ID NO: 5, 7 or 9. A reduced-function variant is a protein that has an amino acid sequence which varies from that of SEQ ID NO: 5, 7 or 9, has the ability to bind the IL-22 receptor and has a reduced/decreased ability to activate or agonise the IL-22 receptor. The function may be reduced/decreased by any amount as discussed above. A non-functional variant is a protein that has an amino acid sequence which varies from that of SEQ ID NO: 5, 7 or 9, has the ability to bind the IL-22 receptor and does not have the ability to activate or agonise the IL-22 receptor. The reduced-function variant of IL-22, such as of SEQ ID NO: 5, typically has the ability to bind IL-22RA1, but has a reduced/decreased ability to bind the IL-10 receptor 2 (IL-10RB2) subunit. The non-function variant of IL-22, such as of SEQ ID NO: 5, typically has the ability to bind IL-22RA1, but does not have the ability to bind the IL-10 receptor 2 (IL-10RB2) subunit. Although such variants bind IL-22RA1, they have a reduced/decreased ability to allow it to heterodimerise with IL-10RB2 or do not allow it to heterodimerise with IL-10RB2. Heterodimerisation is necessary to activate the signal transduction pathways.

The reduced-function variant of IL-20 or IL-24, such as of SEQ ID NO: 7 or 9, typically has the ability to bind IL-22RA1, but has a reduced/decreased ability to bind the IL-20 receptor 2 (IL-20RB2) subunit. The non-functional variant of IL-20 or IL-24, such as of SEQ ID NO: 7 or 9, typically has the ability to bind IL-22RA1, but does not have the ability to bind the IL-20 receptor 2 (IL-20RB2) subunit. Although such variants bind IL-22RA1, they have a reduced/decreased ability to allow it to heterodimerise with IL-20RB2 or do not allow it to heterodimerise with IL-20RB2. Heterodimerisation is necessary to activate the signal transduction pathways.

The reduced-function variant may have a reduced/decreased ability to bind IL-22RA1. The non-functional variant may be unable to bind IL-22RA1.

The comparative binding ability of reduced-function and non-functional variants is typically measured in comparison to wild-type IL-22, IL-20 or IL-24 (such as SEQ ID NO: 5, 7 or 9). Binding can be measured using know techniques, such as those disclosed in Wu et al., Journal of Molecular Biology, Volume 382, Issue 5, 24 Oct. 2008, Pages 1168-1183.

The reduced-function variant may reduce IL-22 signalling by competing with the natural ligands for binding to the IL-22 receptor, but activating the receptor to a lesser degree. The non-functional variant may reduce IL-22 signalling by competing with the natural ligands for binding to the IL-22 receptor, but not activating the receptor. The ability of a variant to bind to and activate or agonise the IL-22 receptor, i.e. bind to IL-22RA1 but not IL-10RB2, can be assayed using any method known in the art. Suitable methods are described above. They are also disclosed in Wu et al., Journal of Molecular Biology, Volume 382, Issue 5, 24 Oct. 2008, Pages 1168-1183.

Over the entire length of the amino acid sequence of SEQ ID NO: 5, 7 or 9, a reduced-function or non-functional variant will preferably be at least 50% homologous to that sequence based on amino acid identity, i.e. have at least 50% amino acid identity over the entire sequence. More preferably, the reduced-function or non-functional variant may be at least 60%, at least 70%, at least 80%, at least 85%, at least 90% and more preferably at least 95%, 97% or 99% homologous based on amino acid identity (or identical) to the amino acid sequence of SEQ ID NO: 5, 7 or 9 over the entire sequence. There may be at least 80%, for example at least 85%, 90% or 95%, amino acid identity over a stretch of 100 or more, for example 200 or 300 or more, contiguous amino acids (“hard homology”).

Amino acid substitutions may be made to the amino acid sequence of SEQ ID NO: 5, 7 or 9, for example up to 1, 2, 3, 4, 5, 10, 20, 30, 50 or 100 substitutions. Conservative substitutions as discussed above may be made.

One or more amino acid residues of the amino acid sequence of SEQ ID NO: 5, 7 or 9 may additionally be deleted from the polypeptides described above. Up to 1, 2, 3, 4, 5, 10, 20, 30 or 50 residues may be deleted, or more.

Reduced-function or non-functional variants may include fragments of SEQ ID NO: 5, 7 or 9. Such fragments typically retain the domain of SEQ ID NO: 5, 7 or 9 which binds IL-22RA1 but lack the domain that binds to IL-10RB2 or IL-20RB2. The IL-10R2 binding site on IL-22 has been localized to the N-terminal end of helix A and N-linked glycosylation on N54 of IL-22 is specifically required for optimal interaction with IL-10RB (Logsdon et al. J Mol Biol. 2004 Sep. 10; 342(2):503-14). Such fragments may lack the domain of SEQ ID NO: 5, 7 or 9 which binds IL-22RA1 but retain the domain that binds to IL-10RB2 or IL-20RB2. Fragments may be at least 200, 300, 400 or 500 amino acids in length. One or more amino acids may be alternatively or additionally added to the polypeptides described above.

A preferred reduced-function variant of IL-22 (SEQ ID NO: 5) is one in which N54 is mutated from asparagine (N) to glutamine (G), i.e. N54G. Preferred reduced-function variants of IL-22 include, but are not limited to, a variant of IL-22 (SEQ ID NO: 5) in which T56 is mutated to alanine (A), i.e. T56A, a variant of IL-22 (SEQ ID NO: 5) in which Y51 is mutated to A, i.e. Y51A, a variant of IL-22 (SEQ ID NO: 5) in which R55 is mutated to alanine (A), i.e. R55A, a variant of IL-22 (SEQ ID NO: 5) in which N54 is mutated to alanine (A), i.e. N54A, a variant of IL-22 (SEQ ID NO: 5) in which F121 is mutated to alanine (A), i.e. F121A, and a variant of IL-22 (SEQ ID NO: 5) in which E117 is mutated to alanine (A), i.e. E117A. These variants are disclosed in Wu et al., Journal of Molecular Biology, Volume 382, Issue 5, 24 Oct. 2008, Pages 1168-1183 and the mutations reduce binding to the IL-22R complex.

Preferred reduced-function variants include, but are not limited to, a variant of IL-22 (SEQ ID NO: 5) in which D67 is mutated to alanine (A), i.e. D67A, a variant of IL-22 (SEQ ID NO: 5) in which V72 is mutated to A, i.e. V72A, a variant of IL-22 (SEQ ID NO: 5) in which 1161 is mutated to alanine (A), i.e. I161A, and a variant of IL-22 (SEQ ID NO: 5) in which K162 is mutated to alanine (A), i.e. K162A. These variants are disclosed in Wu et al., Journal of Molecular Biology, Volume 382, Issue 5, 24 Oct. 2008, Pages 1168-1183 and the mutations reduce binding of the variants IL-22RA1 subunit of the receptor (which is the first step in the binding process).

Alternatively, the inhibitor may be a polynucleotide encoding a reduced-function or non-functional variant of IL-22, IL-20 or IL-24. The reduced-function or non-functional variant may be any of those discussed above. Polynucleotides are defined above.

Inhibitors of IL-22, IL-20 or IL-24 may also reduce amounts of IL-22, IL-20 or IL-24 present in the patient or the cancer, for example by knocking down expression of IL-22, IL-20 or IL-24. Antisense and RNA interference (RNAi) technology for knocking down protein expression are well known in the art and standard methods can be employed to knock down expression of IL-22, IL-20 or IL-24.

Both antisense and siRNA technology interfere with mRNA. Antisense oligonucleotides interfere with mRNA by binding to (hybridising with) a section of the mRNA. The antisense oligonucleotide is therefore designed to be complementary to the mRNA (although the oligonucleotide does not have to be 100% complementary as discussed below). In other words, the antisense oligonucleotide may be a section of the cDNA. Again, the oligonucleotide sequence may not be 100% identical to the cDNA sequence. This is also discussed below.

RNAi involves the use of double-stranded RNA, such small interfering RNA (siRNA) or small hairpin RNA (shRNA), which can bind to the mRNA and inhibit protein expression.

Accordingly, the inhibitor preferably comprises an oligonucleotide which specifically hybridises to a part of the IL-22, IL-20 or IL-24 mRNA. The inhibitor preferably comprises an oligonucleotide which specifically hybridises to a part of SEQ ID NO: 6, 8 or 10 (human IL-22, IL-20 or IL-24 mRNA) or any naturally-occurring variant. The naturally-occurring variant is typically a polymorphism. Over the entire length of the sequence of SEQ ID NO: 6, 8 or 10, a naturally-occurring variant will preferably be at least 90% homologous to that sequence based on nucleotide identity over the entire sequence, i.e. have at least 90% nucleotide identity over the entire sequence. More preferably, the naturally-occurring variant may be at least 95%, 97% or 99% homologous based on nucleotide identity (or identical) to the nucleotide sequence of SEQ ID NO: 6, 8 or 10 over the entire sequence. Homology may be measured as discussed above

Oligonucleotides are short nucleotide polymers which typically have 50 or fewer nucleotides, such 40 or fewer, 30 or fewer, 22 or fewer, 21 or fewer, 20 or fewer, 10 or fewer or 5 or fewer nucleotides. The oligonucleotide used in the invention is preferably 20 to 25 nucleotides in length, more preferably 21 or 22 nucleotides in length. The nucleotides can be naturally occurring or artificial. The nucleotides can be any of those described above.

An oligonucleotide preferably specifically hybridises to a part of SEQ ID NO: 6, 8 or 10 or any naturally-occurring variant thereof as discussed above, hereafter called the target sequence. The length of the target sequence typically corresponds to the length of the oligonucleotide. For instance, a 21 or 22 nucleotide oligonucleotide typically specifically hybridises to a 21 or 22 nucleotide target sequence. The target sequence may therefore be any of the lengths discussed above with reference to the length of the oligonucleotide. The target sequence is typically consecutive nucleotides within the target polynucleotide.

An oligonucleotide “specifically hybridises” to a target sequence as defined above.

The oligonucleotide may comprise a sequence which is substantially complementary to the target sequence. Typically, the oligonucleotides are 100% complementary. However, lower levels of complementarity may also be acceptable, such as 95%, 90%, 85% and even 80%. Complementarity below 100% is acceptable as long as the oligonucleotides specifically hybridise to the target sequence. An oligonucleotide may therefore have 1, 2, 3, 4, 5, 6, 7, 8, 9, 10 or more mismatches across a region of 5, 10, 15, 20, 21, 22, 30, 40 or 50 nucleotides.

Alternatively, the inhibitor preferably comprises an oligonucleotide which comprises 50 or fewer consecutive nucleotides from the reverse complement of (a) SEQ ID NO: 6, 8 or 10 or (b) or any naturally-occurring variant thereof as discussed above. The oligonucleotide may be any of the lengths discussed above. It is preferably 21 or 22 nucleotides in length. The oligonucleotide may comprise any of the nucleotides discussed above, including the modified nucleotides. The olignucleotide may be any of the types discussed above.

The inhibitor is preferably an antibody which specifically binds IL-22, IL-20 or IL-24. The antibody preferably binds the sequence shown in SEQ ID NO: 6, 8 or 10 or a naturally-occurring variant. The naturally-occurring variant is typically a polymorphism. Over the entire length of the amino acid sequence of SEQ ID NO: 6, 8 or 10, a naturally-occurring variant will preferably be at least 90% homologous to that sequence based on amino acid identity, i.e. have at least 90% amino acid identity over the entire sequence. More preferably, the naturally-occurring variant may be at least 95%, 97% or 99% homologous based on amino acid identity (or identical) to the amino acid sequence of SEQ ID NO: 6, 8 or 10 over the entire sequence. Homology may be measured as discussed above.

Specific binding is defined above. The antibody may be any of the types discussed above.

The inhibitor is preferably ILV-094 (Fezakinumab). This is an anti-IL-22 antibody owned by Pfizer®. Other preferred inhibitors are listed in the Tables below.

| | | |
|---|---|---|
| Genentech | anti-IL-22 | U.S. Pat. No 7,737,259 B2 |
| Zymogenetics - | anti-IL-20, anti-IL- | US 20100111960 A1 |
| Bristol Myers Squib | 22, anti-IL-22RA1 | |
| Wyeth Llc, | anti-IL-22 | U.S. Pat. No 7,901,684 B2; |
| Medimmune Ltd | | U.S. Pat. No 8,182,817 B2 |
| Inst Genetic Llc | anti-IL-22 | WO 2002068476 A2 |
| Pfizer | anti-IL-22 | Fezakinumab (ILV-094) |

| | Method of | | | | ClinicalTrials.gov |
|---|---|---|---|---|---|
| Condition | Administration | Phase | Start | Notes | Identifier |
| Atopic | intravenous (IV) | II | August 2013 | | NCT01941537 |
| dermatitis | | | | | |
| Rheumatoid | sub-cutaneous (SC) | II | April 2009 | Discont' | NCT00883896 |
| Arthritis | | | | | |
| Psoriasis | IV | I | November 2007 | Discont' | NCT00563524 |
| Healthy | IV or SC | I | January 2007 | | NCT00434746 |
| Controls | | | | | |
| Healthy | IV or SC | I | March 2007 | | NCT00447681 |
| Controls | | | | | |

### Administration

In the method of the invention, the inhibitor is administered to the patient. The inhibitor of may be administered to the patient in any appropriate way. In the invention, the inhibitor may be administered in a variety of dosage forms. Thus, it can be administered orally, for example as tablets, troches, lozenges, aqueous or oily suspensions, dispersible powders or granules. It may also be administered byenteral or parenteral routes such as via buccal, anal, pulmonary, intravenous, intra-arterial, intramuscular, intraperitoneal, intraarticular, topical or other appropriate administration routes. The inhibitor may be administered directly into the cancer to be treated. The preferred route of administration is intravenous. A physician will be able to determine the required route of administration for each particular patient.

The formulation of the inhibitor will depend upon factors such as the nature of the exact inhibitor, etc. The inhibitor may be formulated for simultaneous, separate or sequential use with other inhibitors defined herein or with other cancer treatments as discussed in more detail below.

The inhibitor is typically formulated for administration with a pharmaceutically acceptable carrier or diluent. The pharmaceutical carrier or diluent may be, for example, an isotonic solution. For example, solid oral forms may contain, together with the active substance, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active substance, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

For suppositories, traditional binders and carriers may include, for example, polyalkylene glycols or triglycerides; such suppositories may be formed from mixtures containing the active ingredient in the range of 0.5% to 10%, preferably 1% to 2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Capsules, tablets and pills for oral administration to an individual may be provided with an enteric coating comprising, for example, Eudragit “S”, Eudragit “L”, cellulose acetate, cellulose acetate phthalate or hydroxypropylmethyl cellulose.

Polynucleotide or oligonucleotide inhibitors maybe naked nucleotide sequences or be in combination with cationic lipids, polymers or targeting systems. They may be delivered by any available technique. For example, the polynucleotide or oligonucleotide may be introduced by needle injection, preferably intradermally, subcutaneously or intramuscularly. Alternatively, the polynucleotide or oligonucleotide may be delivered directly across the skin using a delivery device such as particle-mediated gene delivery. The polynucleotide or oligonucleotide may be administered topically to the skin, or to mucosal surfaces for example by intranasal, oral, or intrarectal administration.

Uptake of polynucleotide or oligonucleotide constructs may be enhanced by several known transfection techniques, for example those including the use of transfection agents. Examples of these agents include cationic agents, for example, calcium phosphate and DEAE-Dextran and lipofectants, for example, lipofectam and transfectam. The dosage of the polynucleotide or oligonucleotide to be administered can be altered.

A therapeutically effective amount of the inhibitor is typically administered to the patient. A therapeutically effective amount of is an amount effective to ameliorate one or more symptoms of the cancer. A therapeutically effective amount of the immunotherapy is preferably an amount effective to abolish one or more of, or preferably all of, the symptoms of the cancer. A therapeutically effective amount preferably leads to a reduction in the size of the cancer or more preferably kills all of the cancer cells.

The dose may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the specific inhibitor, the age, weight and conditions of the subject to be treated and the frequency and route of administration. The dose may be provided as a single dose or may be provided as multiple doses, for example taken at regular intervals, for example 2, 3 or 4 doses administered hourly. Preferably, dosage levels of inhibitors are from 5 mg to 2 g.

Typically polynucleotide or oligonucleotide inhibitors are administered in the range of 1 pg to 1 mg, preferably to 1 pg to 10 μg nucleic acid for particle mediated delivery and 10 μg to 1 mg for other routes.

### Combination Therapy

The inhibitor may be administered in combination with one or more other therapies intended to treat the same patient. A combination means that the therapies may be administered simultaneously, in a combined or separate form, to the patient. The therapies may be administered separately or sequentially to a patient as part of the same therapeutic regimen. For example, an inhibitor may be used in combination with another therapy intended to treat the cancer. The other therapy may be a general therapy aimed at treating or improving the condition of the patient. For example, treatment with methotrexate, glucocorticoids, salicylates, nonsteroidal anti-inflammatory drugs (NSAIDs), analgesics, other DMARDs, aminosalicylates, corticosteroids, and/or immunomodulatory agents (e.g., 6-mercaptopurine and azathioprine) may be combined with the inhibitor. The other therapy may be a specific treatment directed at the cancer suffered by the patient, or directed at a particular symptom of the cancer.

The inhibitor is preferably administered in combination with another cancer therapy. The inhibitor may be used in combination with surgery, such as surgical resection, chemotherapy, radiotherapy or biological therapy. Preferred chemotherapies include, but are not limited to, 5-fluorouracil, irinotecan, leucovorin, oxaliplatin, capecitabine, raltitrexed and combinations thereof. Preferred biological therapies include, but are not limited to, cetuximab, panitumumab, bevacizumab and aflibercept. Although not currently standard of care, additional therapies that may become relevant for colorectal cancer, such as proximal or distal colorectal cancer, in the near future include clinically approved checkpoint blockade immunotherapies, such as nivolumab, pembrolizumab, and ipilimumab. The inhibitor of the invention may be administered in combination with such therapies.

The inhibitor may also be used in combination with a Jak inhibitors, such as tofacitinib, or a STAT3 inhibitor, such as BP-1-102.

Preferred combinations for use in the invention include, but are not limited to, (a) ILV-094 (Fezakinumab) in combination with surgery, such as surgical resection, chemotherapy, such as 5-fluorouracil, irinotecan, leucovorin, oxaliplatin, capecitabine, raltitrexed or combinations thereof, radiotherapy or biological therapy, such as cetuximab, panitumumab, bevacizumab or aflibercept.

### Selection and Treatment

The invention preferably provides a method in which a colorectal cancer patient is selected on the basis of the cancer comprising a KRAS mutation a high amount of IL-22 receptor and then treated in accordance with the invention. The method comprises (a) determining whether or not the cancer comprises a KRAS mutation and measuring the amount of IL-22 receptor in the cancer. The method also comprises (b), if the cancer comprises a KRAS mutation and a high amount of IL-22 receptor, administering to the patient an inhibitor of IL-22 signalling and thereby treating the cancer. Step a) is typically carried out in vitro. This is discussed in more detail below. The colorectal cancer may be distal colorectal cancer. The cancer is preferably proximal colorectal cancer. Step a) is typically carried out using a sample obtained from the cancer, such as cancer biopsy. The sample comprises colorectal cancer cells. Step a) may also be carried out using cancer cells obtained from the patient's blood or from a stool sample from the patient.

The invention preferably provides a method in which a colorectal cancer patient is selected on the basis of the cancer being proximal colorectal cancer and the cancer comprising a KRAS mutation a high amount of IL-22 receptor and then treated in accordance with the invention. The method comprises (a) determining whether or not the cancer is proximal colorectal cancer and (b) comprises a KRAS mutation and measuring the amount of IL-22 receptor in the cancer. The method also comprises (c), if the cancer is proximal colorectal cancer and comprises a KRAS mutation and a high amount of IL-22 receptor, administering to the patient an inhibitor of IL-22 signalling and thereby treating the cancer. Step b) is typically carried out in vitro. This is discussed in more detail below. Step b) is typically carried out using a sample obtained from the cancer, such as cancer biopsy. The sample comprises colorectal cancer cells. Step b) may also be carried out using cancer cells obtained from the patient's blood or from a stool sample from the patient.

Any of the methods discussed above may be used to determine whether or not the cancer comprises a KRAS mutation and to measure the amount of IL-22 receptor in the cancer. Preferred mutations and what is meant by a high amount of IL-22 receptor are discussed above. The patient may be treated in any of the ways discussed above.

### Kits

The present invention also relates to a kit for treating colorectal cancer. The kit comprises means (or reagents) for testing whether or not the cancer comprises a KRAS mutation and for measuring the high amount of IL-22 receptor. The kit thereby allows the determination of whether or not colorectal cancers comprise a KRAS mutation and a high amount of IL-22 receptor. The colorectal cancer may be distal colorectal cancer. The colorectal cancer is preferably proximal colorectal cancer.

The means (or reagent) for testing for whether or not the cancer comprises a KRAS mutation may be any suitable means (or reagent) for the use in the screening methods described above. The means (or reagent) is typically a polynucleotide. The means (or reagent) may comprise sequencing reagents or next generation sequencing reagents.

The means (or reagent) for measuring the amount of IL-22 receptor may be any suitable means or reagent for the use in the screening methods described above. For example, the kit may include an antibody that specifically binds IL-22RA1. The kit may comprise an oligonucleotide which specifically hybridises to part of IL-22RA1 mRNA or cDNA. Oligonucleotides, parts and specific hybridisation are discussed above.

The kit also comprises an inhibitor of IL-22 signalling. The inhibitor may be any of those discussed above.

The kit may additionally comprise one or more other reagents or instruments which enables the method mentioned above to be carried out. Such reagents include means for taking a sample from the patient, suitable buffers, means to extract/isolate polynucleotides or protein from the sample or a support comprising wells on which quantitative reactions can be done. The kit may, optionally, comprise instructions to enable the kit to be used in the method of invention or details regarding patients on which the method may be carried out. The kit may comprise primers and reagents for PCR, qPCR (quantitative PCR), RT-PCR (reverse-transcription PCR), qRT-PCR (quantitative reverse-transcription PCR) reaction or RNA sequencing.

### Prognosis

The invention also provides a method for prognosing colorectal cancer in a patient. The method comprises determining whether or not the cancer comprises a KRAS mutation and measuring the amount of IL-22 receptor in the cancer. The method is typically carried out in vitro. The method is typically carried out using a sample obtained from the cancer, such as a cancer biopsy. The sample comprises colorectal cancer cells. The method may also be carried out using cancer cells obtained from the patient's blood or a stool sample from the patient. Any of the methods discussed above may be used to determine whether or not the cancer comprises a KRAS mutation and to measure the amount of IL-22 receptor in the cancer. Preferred mutations and what is meant by a high amount of IL-22 receptor are discussed above. The colorectal cancer may be distal colorectal cancer. The colorectal cancer is preferably proximal colorectal cancer. The presence of a KRAS mutation and a high amount of IL-22 receptor in the cancer indicates that the patient has a worse prognosis than in the absence of a KRAS mutation and/or in the presence of a low amount of IL-22 receptor. The presence of a KRAS mutation and a high amount of IL-22 receptor in the cancer indicates that the patient has a worse prognosis than in the absence of a KRAS mutation and/or in the absence of a high amount of IL-22 receptor.

The presence of a KRAS mutation and a high amount of IL-22 receptor in the cancer indicates that the patient has a reduced/decreased recurrence-free survival time and/or reduced/decreased overall survival time than in the absence of a KRAS mutation and/or in the presence of a low amount of IL-22 receptor. Recurrence-free survival refers to the period of time following diagnosis during which the patient shows no clinical evidence of disease progression. Overall survival time refers to the time between diagnosis and death from any cause. The presence of a KRAS mutation and a high amount of IL-22 receptor in the cancer preferably indicates that the patient has a recurrence-free five year survival percentage of less than 50% or less than 45% compared with a recurrence-free five year survival percentage of greater than 50%, greater than 60% or greater than 70% in the absence of a KRAS mutation and/or in the presence of a low amount of IL-22 receptor, preferably in the absence of a KRAS mutation and the presence of a high amount of IL-22 receptor. The presence of a KRAS mutation and a high amount of IL-22 receptor in the cancer preferably indicates that the patient has a median recurrence-free five year survival time of less than 60 months, such as less than 50 months, compared with a median recurrence-free five year survival time of greater than 100 months, greater than 110 months or greater than 120 months in the absence of a KRAS mutation and/or in the presence of a low amount of IL-22 receptor, preferably in the absence of a KRAS mutation and the presence of a high amount of IL-22 receptor. The presence of a KRAS mutation and a high amount of IL-22 receptor in the cancer preferably indicates that the patient has a overall five year survival percentage of less than 50% or less than 46% compared with an overall five year survival percentage of greater than 50%, greater than 60% or greater than 70% in the absence of a KRAS mutation and/or in the presence of a low amount of IL-22 receptor, preferably in the absence of a KRAS mutation and the presence of a high amount of IL-22 receptor. The presence of a KRAS mutation and a high amount of IL-22 receptor in the cancer preferably indicates that the patient has a median overall five year survival time of less than 60 months, such as less than 50 months, compared with a median overall five year survival time of greater than 100 months, greater than 110 months or greater than 120 months in the absence of a KRAS mutation and/or in the presence of a low amount of IL-22 receptor, preferably in the absence of a KRAS mutation and the presence of a high amount of IL-22 receptor.

The low amount of IL-22 in the cancer is typically relative to other cancers of the same type, i.e. other colorectal cancers. The low amount of IL-22 in a proximal colorectal cancer is typically relative to other cancers of the same type, i.e. other proximal colorectal cancers. The low amount of IL-22 in a distal colorectal cancer is typically relative to other cancers of the same type, i.e. other distal colorectal cancers. A cancer with a low amount cancer preferably comprises an amount of IL-22 receptor which is less that than the 40^{th} or 33^{rd} percentile of amount in a cohort of colorectal cancers, such as a cohort of proximal colorectal cancers or a cohort of distal colorectal cancers. The cancer may comprise an amount of IL-22 receptor which is lower than the 40^{th}, 39^{th}, 38^{th}, 37^{th}, 36^{th}, 35^{th}, 34^{th}, 33^{rd}, 32^{nd}, 31^{st}, 30^{th}, 29^{th}, 28^{th}, 27^{th}, 26^{th}, 25^{th}, 24^{th}, 23^{rd}, 22^{nd}, 21^{st}, 20^{th}, 19^{th}, 18^{th}, 17^{th}, 16^{th}, 15^{th}, 14^{th}, 13^{th}, 12^{th}, 11^{th}, 10^{th}, 9^{th}, 8^{th}, 7^{th}, 6^{th}, 5^{th}, 4^{th}, 3^{rd}, 2^{nd} or 1^{st} percentile of amount in a cohort of colorectal cancers, such as a cohort of proximal colorectal cancers or a cohort of distal colorectal cancers. The cohort typically comprises at least 10 colorectal cancers, such as at least 20, at least 30, at least 50 or at least 100 colorectal cancers. The percentile of amount can be determined using standard statistical techniques. The cohorts used to determine the high and low amounts are preferably the same.

The low amount of IL-22 receptor is preferably lower than the ratio of the 40^{th} or 33rd percentile (or any of the percentiles listed above) of amount in a cohort of colorectal cancers to the amount of one or more reference (or control) genes. The one or more reference (or control) genes are preferably GPX1, VDAC2, PGK1, ATP5E, and UBB. The amounts of the different genes are preferably measured using the same technique.

The low amount may be a low amount of IL-22RA1 protein and/or a low amount of IL22RA1 mRNA. The low amount of IL-22RA1 protein, such as an amount of IL-22RA1 protein, may be lower than the 40^{th} or 33^{rd} percentile of amount in a cohort of colorectal cancers, such as a cohort of proximal colorectal cancers or a cohort of distal colorectal cancers. The amount may be lower than any of the percentiles of amount discussed above and/or the cohort can have any number of cancers as discussed above.

The low amount may be an amount of IL-22RA1 protein which is lower than the ratio of the 40^{th} or 33^{rd} percentile (or any of the percentiles listed above) of amount in a cohort of colorectal cancers to the amount of one or more reference (or control) proteins. The one or more reference (or control) proteins are preferably GPX1, VDAC2, PGK1, ATP5E, and UBB. The amounts of the different proteins are preferably measured using the same technique. The amount of IL-22RA1 protein can be measured as discussed above.

The low amount may be an amount of IL22RA1 mRNA, such as an amount of IL-22RA1 mRNA which is lower than the 40^{th} or 33^{rd} percentile of amount in a cohort of colorectal cancers, such as a cohort of proximal colorectal cancers or a cohort of distal colorectal cancers. The amount may be lower than any of the percentiles of amount discussed above and/or the cohort can have any number of cancers as discussed above.

The low amount may be an amount of IL-22RA1 mRNA which is lower than the ratio of the 40^{th} or 33^{rd} percentile (or any of the percentiles listed above) of amount in a cohort of colorectal cancers to the amount of one or more reference (or control) mRNAs. The one or more reference (or control) mRNAs are preferably GPX1, VDAC2, PGK1, ATP5E, and UBB. The amounts of the different mRNAs are preferably measured using the same technique. The amount of IL-22RA1 mRNA can be measured as discussed above.

The method preferably comprises determining whether or not the cancer is proximal colorectal cancer, determining whether or not the cancer comprises a KRAS mutation and measuring the amount of IL-22 receptor in the cancer. The presence of a KRAS mutation and a high amount of IL-22 receptor in a proximal colorectal cancer indicates that the patient has a worse prognosis than in the absence of a KRAS mutation and/or in the presence of a low amount of IL-22 receptor. The presence of a KRAS mutation and a high amount of IL-22 receptor in a proximal colorectal cancer indicates that the patient has a worse prognosis than in the absence of a KRAS mutation and/or in the absence of a high amount of IL-22 receptor.

### Responsiveness

The invention also provides a method for determining whether or not a patient with colorectal cancer is likely to (or will) respond to therapy with an inhibitor of IL-22 signalling. The method comprises determining whether or not the cancer comprises a KRAS mutation and measuring the amount of IL-22 receptor in the cancer. The presence of a KRAS mutation and a high amount of IL-22 receptor in the cancer indicates that the patient is likely to (or will) respond to therapy with an inhibitor of IL-22 signalling. The patient may then be treated with any of the inhibitors discussed above. The colorectal cancer may be distal colorectal cancer. The colorectal cancer is preferably proximal colorectal cancer.

The method is typically carried out in vitro. The method is typically carried out using a sample obtained from the cancer, such as cancer biopsy. The sample comprises proximal colorectal cancer cells. The method may also be carried out using cancer cells obtained from the patient's blood or a stool sample from the patient. Any of the methods discussed above may be used to determine whether or not the cancer comprises a KRAS mutation and to measure the amount of IL-22 receptor in the cancer. Preferred mutations and what is meant by a high amount of IL-22 receptor are discussed above.

The method preferably comprises determining whether or not the cancer is proximal colorectal cancer, whether or not the cancer comprises a KRAS mutation and measuring the amount of IL-22 receptor in the cancer. The presence of a KRAS mutation and a high amount of IL-22 receptor in a proximal colorectal cancer indicates that the patient is likely to (or will) respond to therapy with an inhibitor of IL-22 signalling.

### In Vitro Assay

The invention also provides an in vitro assay for determining whether or not a patient with colorectal cancer is likely to (or will) respond to therapy with an inhibitor of IL-22 signalling. The assay comprises determining whether or not a sample from the cancer comprises a KRAS mutation and measuring the amount of IL-22 receptor in the cancer. The presence of a KRAS mutation and a high amount of IL-22 receptor in the sample indicates that the patient is likely to (or will) respond to therapy with an inhibitor of IL-22 signalling.

The invention also provides an in vitro assay for prognosing colorectal cancer in a patient. The assay comprises determining whether or not a sample from the cancer comprises a KRAS mutation and measuring the amount of IL-22 receptor in the cancer. The presence of a KRAS mutation and a high amount of IL-22 receptor in the cancer indicates that the patient has a worse prognosis than in the absence of a KRAS mutation and/or in the presence of a low amount of IL-22 receptor.

The colorectal cancer may be distal colorectal cancer. The colorectal cancer is preferably proximal colorectal cancer.

The assay is typically carried out on a sample obtained from the cancer, such as a cancer biopsy. The sample or biopsy comprises colorectal cancer cells. The assay may comprise cancer cells obtained from the patient's blood or a stool sample from the patient.

Any of the methods discussed above may be used to determine whether or not the cancer comprises a KRAS mutation and to measure the amount of IL-22 receptor in the cancer. The assay may make use of any means (reagents) needed to perform the relevant determinations and measurements, such as one or more polynucleotides or oligonucleotides and/or one or more antibodies. Preferred mutations and what is meant by a high amount of IL-22 receptor are discussed above.

The sample may contain any number of cells, such as at least 1,000 cells, such as at least 5,000 cells or at least 10,000 cells.

The assay may be carried out in any suitable volume. Typical volumes range from about 10 μl to about 1 ml, preferably from about 50 μl to about 500 μl, more preferably from about 100 μl to about 200 μl.

The assay may be carried out at any suitable temperature. The suitable temperature is typically in the same range as the normal body temperature of the human or animal from which the cells are derived. Typically, the incubation is carried out at a fixed temperature between about 4° C. and about 38° C., preferably at about 37° C.

Techniques for culturing cells are well known to a person skilled in the art. The cells are typically cultured under standard conditions of 37° C., 5% CO₂ in medium supplemented with serum.

The method may be carried out using any number of samples from any number of patients. For instance, the method may be carried out using 1, 2, 5, 10, 15, 20, 30, 40, 50, 100, 150, 200, 300, 500 or more samples. The method is preferably carried out using 6, 12, 24, 48, 96 or 384 or 1526 samples. Two or more samples may be from the patient. Alternatively, each sample may be from a different patient. This allows high-throughput screening.

The cancer cells in the sample are preferably captured or immobilized on a surface. Any method of immobilizing or capturing the cells can be used. The cells may be immobilized or captured on the surface using Fc receptors, capture antibodies, avidin:biotin, lectins, polymers or any other capture chemicals.

The one or more samples are typically present in wells. The samples are preferably present in the wells of a flat plate. The samples are more preferably present in the wells of a standard 96 or 384 well plate.

The assay comprises determining whether or not a sample from the cancer comprises a KRAS mutation and measuring the amount of IL-22 receptor in the cancer. The presence of a KRAS mutation and a high amount of IL-22 receptor in the sample indicates that the patient is likely to (or will) respond to therapy with an inhibitor of IL-22 signalling. In terms of a KRAS mutation, the sample either comprises a mutation or does not. The presence of a high amount of IL-22 receptor requires a comparison, typically with the amounts in other cancers in a cohort of proximal colorectal cancers, such as a cohort of proximal colorectal cancers or a cohort of distal colorectal cancers. In one embodiment, the amounts of IL-22 receptor in the other cancers in the cohort is obtained separately from the method of the invention. For instance, the amounts in the other cancers in the cohort may be obtained beforehand and recorded, for instance on a computer.

In another embodiment, the amount of IL-22 receptor in the sample from the patient is obtained at the same time as the amounts of IL-22 receptor in the other cancers in the cohort. This is straightforward to do if the samples from all of the cancers in the cohort are present in the wells of a standard 96 or 384 well plate. This is advantageous because the samples are then assayed using the same conditions.

### System

The invention also provides a system for determining whether or not a patient with colorectal cancer is likely to (or will) respond to therapy with an inhibitor of IL-22 signalling. The system comprises

(a) a measuring module for determining whether or not the cancer comprises a KRAS mutation and for measuring the amount of IL-22 receptor in the cancer,

(b) a storage module configured to store control data and output data from the measuring module,

(c) a computation module configured to provide a comparison between the value of the output data from the measuring module and the control data; and

(d) an output module configured to display whether or not the patient is likely to (or will) respond to therapy with an inhibitor of IL-22 signalling based on the comparison.

The presence of a KRAS mutation and a high amount of IL-22 receptor in the cancer indicates that the patient is likely to (or will) respond to therapy with an inhibitor of IL-22 signalling.

The invention also provides a system for prognosing colorectal cancer in a patient. The system comprises

(a) a measuring module for determining whether or not the cancer comprises a KRAS mutation and for measuring the amount of IL-22 receptor in the cancer,

(b) a storage module configured to store control data and output data from the measuring module,

(c) a computation module configured to provide a comparison between the value of the output data from the measuring module and the control data; and

(d) an output module configured to display the patient's prognosis based on the comparison.

The presence of a KRAS mutation and a high amount of IL-22 receptor in the cancer indicates that the patient has a worse prognosis than in the absence of a KRAS mutation or in the presence of a low amount of IL-22 receptor.

The colorectal cancer may be distal colorectal cancer. The colorectal cancer is preferably proximal colorectal cancer.

Any of the embodiments discussed above with reference to determining whether or not the cancer comprises a KRAS mutation and for measuring the amount of IL-22 receptor in the cancer equally apply to the systems of the invention. The control data in the storage module typically comprises one or more of, such as all of, (a) the sequence of the wild-type (or native) KRAS protein and/or KRAS polynucleotide, (b) a list of KRAS mutations and/or mutated KRAS sequences and (c) the amounts of IL-22 receptor in other colorectal cancers, such as other proximal colorectal cancers or other distal colorectal cancers. The control data may comprise (a); (b); (c); (a) and (b); (a) and (c); (b) and (d); or (a), (b) and (c).

The measuring module in (a) may comprises any of the features of the in vitro assay of the invention. Modules (b) to (d) are typically on a computer.

### Example

### Summary

Interleukin 22 (IL-22) is a cytokine that may promote colorectal cancer (CRC) progression based on human and murine preclinical data. However, the clinical relevance of IL-22 in CRC remains unexplored. Because Ras is a component of IL-22 signaling, we investigated the pre-specified hypothesis that IL-22 promotes disease progression in CRC patients in a manner dependent on KRAS mutation status.

We assessed pre-therapeutic IL22RA1 (IL-22 receptor) expression in CRC specimens using transcriptome profiling data from a population-based French cohort (GSE39582, n=469) as a training dataset. Findings were validated using gene expression data from the PETACC3 (NCT00026273) clinical trial (n=752) and three additional independent cohorts (TCGA, n=X; ALMAC, n=X; and GSE14333, n=1820). Interactions between clinical outcome, KRAS mutation, and IL22RA1 expression were assessed using Cox proportional hazard models.

In tumors with high expression of IL22RA1 in the training cohort, KRAS mutation was significantly associated with poor recurrence-free (HR=2.93, P=0.0006), and overall survival (HR=2.45, P=0.0023). In contrast, KRAS did not associate with prognosis in IL22RA1-low tumors. Similar results were obtained when cases were stratified by expression of IL10RB, the second component of the IL-22 receptor complex. The interaction between IL22RA1 expression, KRAS mutation, and prognosis was detectable preferentially in proximal (right-sided) tumors. All major findings were replicable in the validation cohorts.

We have identified a novel poor-prognosis CRC subtype defined by proximal location, high IL22RA1 expression, and KRAS mutation. The poor prognosis associated with KRAS mutation is strongly dependent on high expression of IL-22 receptor subunits. This provides useful information for identifying patients with KRAS-mutant tumors who may be resistant to standard therapies. Furthermore, this patient subgroup may be sensitive to therapeutic IL-22 blockade.

### Patients and Methods

### Description of Transcriptomic Datasets

GSE39582 (Marisa et al. PLos Medicine, 2013)²⁴ (N=469) also referred to as the French cohort.
(HG-U133A Affymetrix platform)
GSE39582 dataset contains only one probe that detects IL22RA1 (220056_at).
KRAS mutations represented in cohort (G12A, G12C, G12D, G12S, G12V, G13D)

### Validation Cohort

PETACC3 (Pan-European Trials in Alimentary Tract Cancers; NCT00026273)
A set of 752 colorectal cancer patients of stage II (108/752) and stage III (644/752) was used from the PETACC-3 clinical trial. ^{25,26} PETACC3 is a randomized phase III adjuvant chemotherapy trial investigating the efficacy of irinotecan added to fluorouracil (FU)/leucorovin (FA). Gene expression from PETACC-3 patients was obtained using the ALMAC Colorectal Cancer DSA platform (Craigavon, Northern Ireland), which is a customized Affymetrix chip that includes 61,528 probe sets mapping to 15,920 unique Entrez Gene IDs.
PETACC3 dataset contains three different probes for IL22RA1. The probe used in analysis was that which displayed the greatest variation in the dataset (ADXCRAD_BX089163_s_at)

Merged Dataset is constituted from stage II and III patients of GSE39582,²⁴ PETACC3,^{25,26} TCGA²⁷ and ALMAC²⁸ and represents 1820 patients. The ALMAC dataset was obtained from ArrayExpress (www.ebi.ac.uk/arrayexpress) on the A-AFFY-101 platform (customized Affymetrix chip) and is a merge of E-MTAB-863 and E-MTAB-864.²⁸ Clinical information on overall survival was available for 1734 patients and on relapse-free survival for 1499 patients.
Gene expression profiles of the datasets were merged at the gene level, then normalized and corrected for batch effect using the Combat R package.
For the individual analyses of the GSE39582 and PETACC3 datasets, gene expression profiles from each of the datasets were used independently (not normalized to the others).
Definition of proximal and distal tumors in the datasets. Tumors proximal to the splenic flexure were defined as proximal and tumors distal to the splenic flexure were classified as distal.

### Statistical Analysis

All analyses were performed using the R software (version 3.03). Receiver Operating Characteristic ROC analysis was performed to determine the IL22RA1 cutpoint based on log 2 expression values in the training cohort (GSE39582). This cutpoint was used to define the high and low IL22RA1 expression in the validation cohorts. Contingency analysis (Fisher's exact test) was used to assess association of clinical pathological features with IL22RA1 expression status. Probabilities associated with Fisher's exact test were corrected for multiple comparisons using the Bonferroni method. Univariate, (multivariate) and interaction analyses of relapse-free survival (RFS) and overall survival (OS) were performed using Cox's proportional hazard regression models using the survival R package. Interaction analyses were used to assess specific interaction between KRAS mutation status and the expression of interleukin genes and receptors. Hazard ratios (HRs) were estimated with model coefficients and 95% confidence intervals (CIs) and P values were computed with Wald tests. Time-to-event curves were prepared using Kaplan-Meier methods.

### Cell Culture

Colo205, LS1034, SW948, SW480, T84, and HCT116 (ATCC) colorectal cancer cell (CRC) lines were a generous gift from Dr. Simon Leedham and were confirmed to be mycoplasma free. X-MAN DLD-1 isogenic cells were purchased from Horizon Discovery. Colo205, LS1034, LIM1863, and DLD-1 cells were cultured in RPMI with 10% FBS, 100 U/mL each penicillin and streptomycin (P/S). SW948, SW480, and HCT116 cells were maintained in DMEM with 10% FBS, 100 U/mL P/S. T84 cells were cultured in DMEM F12 Hams (Sigma D8437 DMEM Nutrient Mix F-12) with 5% FBS, 100 U/mL P/S. Cultures were maintained in 37° C., 5% CO₂. For basic cytokine stimulation assays 3×10⁴ cells/well were seeded into 48 well plates overnight, before addition of cytokines. Cells were stimulated for 24 h with 1 ng/mL or 10 ng/mL recombinant human IL-22, IL-6, or TNFα (R&D Systems). Following 24 h stimulation cells were used for qPCR or Western Blot analysis.

RNA Extraction and qPCR

RNA was extracted from cultured monolayers of CRC lines, suspensions of LIM1863 spheroids, and CRC line derived spheres using the RNeasy Mini Kit (Qiagen) according to the manufacturers protocol. cDNA was synthesized using the High-Capacity cDNA Reverse Transcription Kit (Applied Biosystems). Gene expression was analyzed using Taqman® Gene Expression Assays (Applied Biosystems) and run with Precision 2× Master Mix (Primerdesign) in 384 well plates using the ViiA7 Real-Time PCR System (Applied Biosystems). Raw Ct values were analyzed using the ΔCt method with RPLPO as an endogenous control to compare relative levels of gene expression between lines or the ΔΔCt method normalized to RPLPO and the untreated condition in a given cell line to measure fold changes in gene expression within a line.

### Immunoblotting

Protein was extracted from adherent CRC cell monolayers or LIM1863 spheroids in suspension using a solution of 50 mM Tris pH 6.8, 20 mM EDTA, 5% SDS, 1 mM DTT, 10% glycerol. 15 ug of cell lysate was loaded into pre-cast NuPAGE Novex 4-12% Bis-Tris Gels (Life Technologies), separated by SDS-PAGE and transferred onto PVDF membrane using a wet transfer apparatus. Non-specific background binding was blocked with 5% Skim Milk in Tris-HCl buffer containing 0.05% Tween-20 (TBST). Membranes were incubated with primary antibody: anti-pSTAT3-Ser727 (9134P, 1:1000 dilution, Cell Signaling), anti-pERK1/2 (4370S, 1:1000 dilution, Cell Signaling), p-Akt (4060S, 1:1000 dilution, Cell Signaling), total ERK 1/2 (4695S, 1:1000 dilution, Cell Signaling), anti-OLFM4 (ab85046, 1:1000 dilution, Abcam), anti-β actin (4967, 1:10,000 dilution, Cell Signaling) in 5% BSA, TBST, washed with TBST, and incubated with HRP conjugated secondary antibody for 1 h at room temperature. Protein expression was detected using Pierce ECL Plus (Thermo Scientific).

### Flow Cytometry

Adherent Colo205, T84, SW480, and DLD-1 cells were dissociated using StemPro® Accutase® (Life Technologies), filtered through 70 μM filters, and counted by Trypan Blue exclusion. 5×10⁵ cells of each line were stained with 5 μL Human IL-22 R alpha 1 Phycoerythrin MAb (Clone 305405) (R&D Systems) or 24 isotype rat anti-mouse IgG1 PE (BD Biosciences) and incubated for 30 minutes at room temperature. Cells were washed with PBS, 0.1% BSA, 2 mM EDTA and acquired on the BD LSRII. Analysis was performed using FlowJo (Tree Star) software. For analysis of intracellular signaling pathways by phosflow, DLD-1 isogenic cells were plated in 6 well plates (1.5 million cells/well) in serum free RPMI and allowed to adhere overnight. DLD-1 cells were then stimulated or not for 30 minutes with increasing doses of recombinant human IL-22 (0.001 ng/mL-100 ng/mL). Cells were dissociated with TrypLE, fixed for 10 min at 37° C. with BD Cytofix Fixation Buffer. Cells were then permeabilized in BD Phosflow Perm Buffer III for 30 min on ice. Cells were then washed three times in PBS, 0.1% BSA, 2 mM EDTA and stained with either Alexa Fluor 647 mouse anti-STAT3(pY705), Alexa Fluor 647 mouse anti-ERK1/2(T202/Y204), or Alexa Fluor 488 Mouse anti-S6(pS235/pS236) at concentration of 1:10 for each antibody for 1 h at room temperature. Cells were washed with PBS, 0.1% BSA, 2 mM EDTA and acquired on the BD Fortessa. Analysis was performed using FlowJo (Tree Star) software).

### Chemotherapy Killing Assay

Colo205, T84, SW480, and DLD-1 cells were seeded at a density of 1×10⁴ cells/well in 48 well plates. Cells were pre-treated for 48 h with 10 ng/mL IL-22, then subjected to 50 μM oxaliplatin (Sigma) or 5-fluorouracil (Sigma) or vehicle control (DMSO) for 48 h. 50 μg Methylthiazolyldiphenyl-tetrazolium bromide (MTT) (Sigma) was added to each well 2 h prior to the end of incubation. At the end of the 48 h incubation supernatants were aspirated and formazan particles were solubilized with DMSO and transferred to a fresh plate. Absorbance was measured at 540 nm on Spectrostar Nano plate reader (BMG Labtech).

### Sphere Forming Assays

Colo205, T84, and SW480 cells were pretreated for 48 h in 48 well plates with 10 ng/mL IL-22 (R&D Systems). Cells were filtered to single cells using 70 μM filter and 1000 cells/well were seeded into 96 well low binding plates (Corning) in 1% methylcellulose in IMDM (R&D Systems), 20 ng/mL recombinant EGF (Sigma), 20 ng/mL recombinant basic FGF (Peprotech), lx insulin-transferrin selenite (ITS) (Sigma) in serum-free DMEM. 10 ng/mL IL-22 was also added in continuous stimulation conditions. Cells were incubated for 6 days in 37° C., 5% CO₂. Bright field images were taken of each well at 4× and spheres were enumerated using the Edge Detection and Analyze Particle functions on ImageJ. Spheres with perimeters under 1.0 pixels were excluded from the computation.

### Statistical Analysis

Data were analyzed for statistical significance using Prism 6.0d (GraphPad). All data are represented as means+SEM with at least 3 independent experiments and 2-4 experimental replicates. One-way ANOVA with Tukey post test for multiple comparisons used for comparison of each group to all other groups and Dunnett's post test for multiple comparisons used for comparison of each group to the control condition. Values of p<0.05 were considered significant.

### Results

### KRAS Mutation is Prognostic in Patients Whose Tumors Express High Levels of the Interleukin 22 Receptor

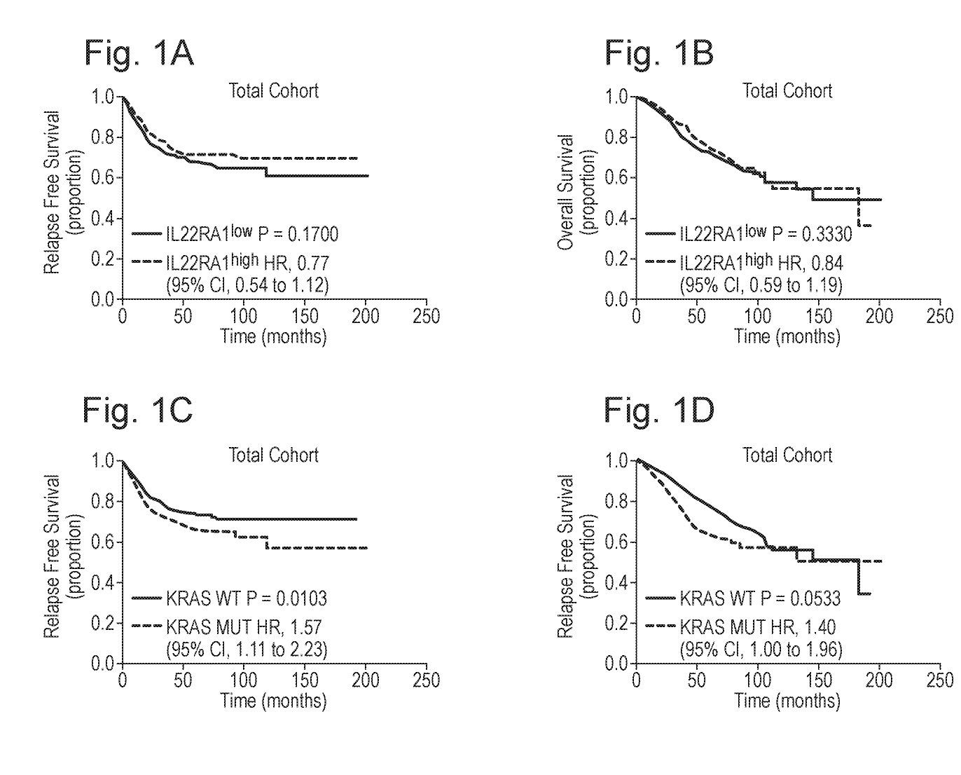

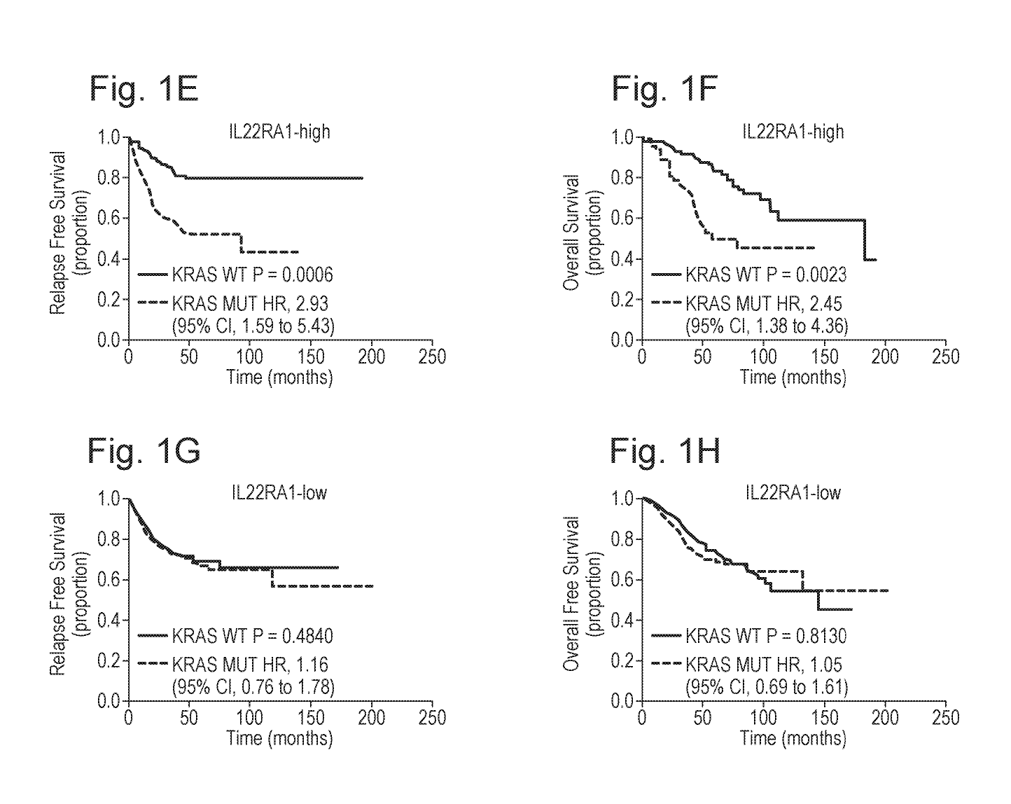

To determine if tumor sensitivity to IL-22 is associated with clinical outcome, we first stratified stage II and III patients (n=469) in the discovery cohort (GSE39582) based on high (top 33%) or low IL22RA1 mRNA expression. This cutpoint was determined using ROC analysis. IL22RA1 expression had no significant impact on relapse-free survival (RFS) or overall survival (OS) in the whole cohort (FIG. 1A,B). Consistent with prior reports,¹² activating KRAS mutations were weakly associated with poor clinical outcome (FIG. 1C,D). However, among cases with high IL22RA1 expression, KRAS mutations were strongly associated with both poor RFS (HR=2.93, 95% CI=1.59-5.43, P=0.0006) (FIG. 1E) and OS (HR=2.45, 95% CI=1.38-4.36, P=0.0023) (FIG. 1F). In contrast, KRAS mutation status had no prognostic impact in patients with IL22RA1-low tumors (RFS HR=1.16, 95% CI=0.76-1.78, P=0.4840; OS HR=1.05, 95% CI=0.69-1.61, P=0.813) (FIG. 1G,H). This association between IL22RA1 and KRAS was validated in a confirmation cohort of randomized stage II and III CRC patients (n=752) enrolled in the PETACC3 clinical trial (Table 1). Finally, in a merged dataset comprised of stage II/III patients from the GSE39582, PETACC3, TCGA, and ALMAC datasets (n=1533), the negative prognostic effect of KRAS mutation in patients with IL22RA1-high tumors was profound (RFS HR=2.05, 95% CI=1.45-2.89, P<0.0001; OS HR=2.07, 95% CI=1.44-2.96, P=0.0001) (Table 1). Furthermore, a significant interaction between IL22RA1 status (high/low) and KRAS status (wild type/mutant) was detected for both RFS and OS in the merged dataset (RFS HR=1.76, 95% CI=1.17 to 2.63; P=0.007; OS HR=1.65, 95% CI=1.09-2.50; P=0.018).

TABLE 1
Survival anaylysis (RFS and OS) for Stage II, III patients of
GSE39582, PETACC3 and combined dataset (univariate analysis)

| | RFS | | | OS | | |
|---|---|---|---|---|---|---|
| No. | P | HR | 95% CI | P | HR | 95% CI |

GSE39582

| | | | | | | | |
|---|---|---|---|---|---|---|---|
| IL22RA1^{high}/IL22RA1^{low} | 157/312 | 0.1700 | 0.77 | 0.54 to 1.12 | 0.3330 | 0.81 | 0.59 to 1.19 |
| KRAS mut/KRAS WT | 169/279 | 0.0103 | 1.57 | 1.11 to 2.23 | 0.0533 | 1.40 | 1.00 to 1.96 |
| Within IL22RA1 [symbol] KRAS mut/KRAS WT | 120/176 | 0.4840 | 1.16 | 0.76 to 1.78 | 0.8130 | 1.05 | 0.69 to 1.61 |
| Within IL22RA1 [symbol] KRAS mut/KRAS WT | 49/103 | 0.0006 | 2.93 | 1.59 to 5.43 | 0.0023 | 2.45 | 1.38 to 4.36 |
| Within KRAS WT IL22RA1 [symbol] IL22RA1 [symbol] | 103/176 | 0.0365 | 0.57 | 0.34 to 0.97 | 0.0650 | 0.64 | 0.40 to 1.03 |
| Within KRAS mut IL22RA1 [symbol] /IL22RA1 [symbol] | 49/120 | 0.1860 | 1.43 | 0.84 to 2.42 | 0.1930 | 1.43 | 0.84 to 2.43 |

PETACC3

| | | | | | | | |
|---|---|---|---|---|---|---|---|
| IL22RA1 [symbol] /IL22RA1 [symbol] | 247/505 | 0.4160 | 1.11 | 0.86 to 1.43 | 0.7300 | 0.95 | 0.70 to 1.28 |
| KRAS mut/KRAS WT | 283/425 | 0.0215 | 1.34 | 1.04 to 1.72 | 0.0051 | 1.51 | 1.13 to 2.02 |
| Within IL22RA1 [symbol] KRAS mut/KRAS WT | 194/278 | 0.2660 | 1.19 | 0.87 to 1.63 | 0.1260 | 1.32 | 0.92 to 1.88 |
| Within IL22RA1 [symbol] KRAS mut/KRAS WT | 89/147 | 0.0149 | 1.68 | 1.11 to 2.56 | 0.0070 | 2.00 | 1.21 to 3.30 |
| Within KRAS WT: IL22RA1 [symbol] /IL22RA1 [symbol] | 147/278 | 0.8800 | 0.97 | 0.88 to 1.39 | 0.3890 | 0.83 | 0.54 to 1.27 |
| Within KRAS mut: IL22RA1 [symbol] /IL22RA1 [symbol] | 89/194 | 0.1040 | 1.38 | 0.94 to 2.02 | 0.3670 | 1.22 | 0.79 to 1.89 |

Combined

| | | | | | | | |
|---|---|---|---|---|---|---|---|
| IL22RA1^{high}/IL22RA1 [symbol] | 590/1230 | 0.9200 | 0.99 | 0.83 to 1.19 | 0.4170 | 0.93 | 0.77 to 1.12 |
| KRAS mut/KRAS WT | 515/881 | 0.0006 | 1.43 | 1.16 to 1.75 | 0.0054 | 1.35 | 1.09 to 1.66 |
| Within IL22RA1 [symbol] KRAS mut/KRAS WT | 361/570 | 0.1800 | 1.19 | 0.92 to 1.53 | 0.5030 | 1.09 | 0.84 to 1.42 |
| Within IL22RA1 [symbol] KRAS mut/KRAS WT | 154/311 | 0.0000 | 2.05 | 1.45 to 2.89 | 0.0001 | 2.07 | 1.44 to 2.96 |
| Within KRAS WT: IL22RA1 [symbol] /IL22RA1 [symbol] | 311/570 | 0.1390 | 0.80 | 0.60 to 1.07 | 0.0468 | 0.74 | 0.55 to 1.00 |
| Within KRAS mut: IL22RA1 [symbol] /IL22RA1 [symbol] | 154/361 | 0.0465 | 1.37 | 1.00 to 1.87 | 0.0660 | 1.36 | 0.98 to 1.89 |

Table 1 KRAS mutation dramatically worsens prognosis in patients with IL22RA1^{high} tumours. Univariate survival analysis of Stage II/III GSE39582 training set, PETACC3 and combined cohort validation sets. Effect of KRAS mutation status stratified according to IL22RA1 expression (high/low). Cox proportional Hazard analyses were performed on overall survival and relapse free survival. The hazard ratio, 95% confidence intervals, and associated Wald p-values are displayed. Significant results are highlighted in bold.
Abbreviations: RFS, relapse free survival; OS, overall survival; HR, hazard ratio; mut, mutant; WT, wild type. [symbol]
indicates data missing or illegible when filed

Unbiased Screen for Interleukins and Interleukin Receptors that Interact with KRAS Mutation

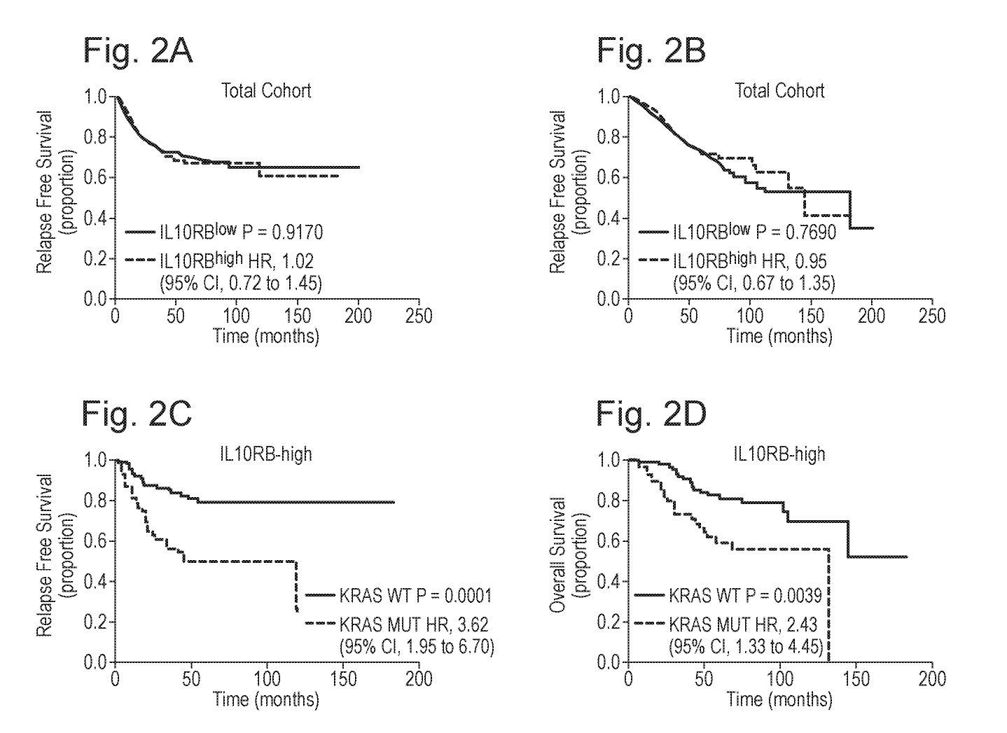

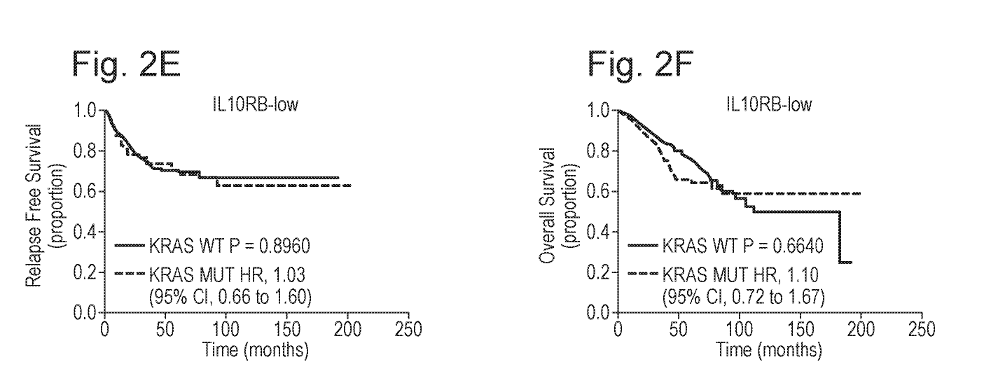

Evidence from mouse models informed our specific interrogation of IL22RA1 and its synergy with oncogenic Ras in the clinical cohorts. Interleukin 6 (IL-6) has a well-documented role in CRC^{18,19} and drives similar signal transduction pathways to IL-22. However, no significant interaction between IL6R call (high/low) and KRAS status (wild type/mutant) was detectable (Table S1). To determine whether other interleukins and/or their cognate receptors stratify KRAS mutations in terms of patient survival, a Cox proportional hazards interaction analysis was performed on all interleukin/interleukin receptor genes (classifying the highest expression tertile for each gene as ‘high’) and KRAS mutation status in the combined cohort. While several other genes interacted with KRAS mutation, the strongest hit was IL22RA1 (Table S2). Remarkably, the second most significant interactor was IL10RB, which encodes the IL-10 receptor 2 protein and is the second subunit of the heterodimeric IL-22 receptor. Detailed survival analysis in the GSE39582 cohort revealed that like IL22RA1, patients whose tumours were IL10RB-high and KRAS-mutant had dramatically worsened prognosis compared to their wild type counterparts (RFS, HR=3.62, 95% CI=1.95-6.70, P<0.0001; OS, HR=2.43, 95% CI=1.33-4.45, P=0.0039). This was confirmed in the combined cohort, although it did not reach significance in the PETACC3 cohort alone (FIG. 2, Table 3). The prognostic implication of having high expression of both subunits of the IL-22 receptor in KRAS mutant tumours underscores a functional synergy between signaling downstream of the IL-22R and oncogenic KRAS that promotes malignant progression.

TABLE S1
Results of unbiased interaction coxph survival analysis between cytokines/
cytokine receptors and KRAS mutation in combined dataset

| | RFS | | | OS | | |
|---|---|---|---|---|---|---|
| Name | P | HR | 95% CI | P | HR | 95% CI |
| IL22RA1 * KRAS mut | 0.0065 | 1.76 | 1.17 to 2.63 | 0.0179 | 1.65 | 1.09 to 2.50 |
| IL10RB * KRAS mut | 0.0177 | 1.62 | 1.09 to 2.41 | 0.0463 | 1.51 | 1.00 to 2.28 |
| IL13RA2 * KRAS mut | 0.0403 | 0.64 | 0.42 to 0.98 | 0.2549 | 0.78 | 0.51 to 1.20 |
| IL3RA * KRAS mut | 0.0489 | 1.51 | 1.00 to 2.27 | 0.0731 | 1.46 | 0.97 to 2.20 |
| IL1RAP * KRAS mut | 0.1176 | 0.71 | 0.46 to 1.09 | 0.0437 | 0.64 | 0.42 to 0.99 |
| IL17RD * KRAS mut | 0.1593 | 0.75 | 0.50 to 1.12 | 0.0044 | 0.55 | 0.36 to 0.83 |
| IL4R * KRAS mut | 0.3131 | 1.23 | 0.82 to 1.83 | 0.7395 | 1.07 | 0.72 to 1.60 |
| IL32 * KRAS mut | 0.5130 | 1.15 | 0.75 to 1.76 | 0.0362 | 1.58 | 1.03 to 2.42 |

Table S1. Unbiased screen for cytokines and cytokine receptors that interact with KRAS and impact survival. Interleukins and interleukin receptors that interact with KRAS mutation status in combined dataset. Univariate Cox proportional Hazard interaction analyses were performed on overall survival and relapse free survival. The hazard ratio, 95% confidence intervals, and associated Wald p-values are displayed. Significant results are highlighted in bold.
Abbreviations: RFS, relapse free survival; OS, overall survival; HR, hazard ratio; mut, mutant; WT, wild type.

TABLE S2
Survival analysis (RFS and OS) for combined dataset (univariate analysis)

| | RFS | | | OS | | |
|---|---|---|---|---|---|---|
| Name | P | HR | 95% CI | P | HR | 95% CI |
| Within IL22RA1^{low}: KRAS mut/KRAS WT | 0.18000 | 0.84 | 0.66 to 1.08 | 0.50300 | 1.09 | 0.84 to 1.42 |
| Within IL22RA1^{high}: KRAS mut/KRAS WT | 0.00005 | 2.05 | 1.45 to 2.89 | 0.00008 | 2.07 | 1.44 to 2.96 |
| Within IL10RB^{low}: KRAS mut/KRAS WT | 0.12500 | 1.22 | 0.95 to 1.58 | 0.17100 | 1.20 | 0.92 to 1.56 |
| Within IL10RB^{high}: KRAS mut/KRAS WT | 0.00017 | 1.89 | 1.36 to 2.63 | 0.00267 | 1.73 | 1.21 to 2.48 |
| Within IL13RA2^{low}: KRAS mut/KRAS WT | 0.00002 | 1.69 | 1.33 to 2.15 | 0.00125 | 1.51 | 1.18 to 1.94 |
| Within IL13RA2^{high}: KRAS mut/KRAS WT | 0.81300 | 0.95 | 0.65 to 1.40 | 0.89000 | 1.03 | 0.69 to 1.53 |
| Within IL3RA^{low}: KRAS mut/KRAS WT | 0.11600 | 1.22 | 0.95 to 1.57 | 0.18200 | 1.19 | 0.92 to 1.55 |
| Within IL3RA^{high}: KRAS mut/KRAS WT | 0.00021 | 1.92 | 1.36 to 2.71 | 0.00336 | 1.71 | 1.19 to 2.44 |
| Within IL1RAP^{low}: KRAS mut/KRAS WT | 0.00096 | 1.50 | 1.18 to 1.90 | 0.00435 | 1.45 | 1.12 to 1.86 |
| Within IL1RAP^{high}: KRAS mut/KRAS WT | 0.23800 | 1.26 | 0.86 to 1.86 | 0.42500 | 1.17 | 0.80 to 1.72 |
| Within IL17RD^{low}: KRAS mut/KRAS WT | 0.00041 | 1.57 | 1.22 to 2.02 | 0.00006 | 1.68 | 1.30 to 2.16 |
| Within IL17RD^{high}: KRAS mut/KRAS WT | 0.38900 | 1.16 | 0.82 to 1.65 | 0.48500 | 0.87 | 0.60 to 1.28 |
| Within IL4R^{low}: KRAS mut/KRAS WT | 0.05210 | 1.29 | 1.00 to 1.66 | 0.07580 | 1.27 | 0.98 to 1.85 |
| Within IL4R^{high}: KRAS mut/KRAS WT | 0.00142 | 1.73 | 1.23 to 2.41 | 0.02300 | 1.50 | 1.06 to 2.13 |
| Within IL32^{low}: KRAS mut/KRAS WT | 0.01920 | 1.34 | 1.05 to 1.70 | 0.23700 | 1.16 | 0.90 to 1.50 |
| Within IL32^{high}: KRAS mut/KRAS WT | 0.01350 | 1.59 | 1.10 to 2.31 | 0.00180 | 1.84 | 1.26 to 2.71 |

Table S2. IL22RA1 and IL10RB have the strongest survival effect in unbiased screen for cytokines and cytokine receptors that interact with KRAS and impact survival. Univariate survival analysis of combined dataset. Effect of KRAS mutation status stratified according to expression level of interleukins and interleukin receptors found to interact with KRAS. Expression values above the 67^{th} percentile in the total cohort were categorized as high. Cox proportional Hazard analyses were performed on overall survival and relapse free survival. The hazard ratio, 95% confidence intervals, and associated Wald p-values are displayed. Significant results are highlighted in bold.
Abbreviations: RFS, relapse free survival; OS, overall survival; HR, hazard ratio; mut, mutant; WT, wild type.

TABLE 3
Survival analysis (RFS and OS) for Stage II, III patients of
GSE39582, PETACC3 and combined dataset (univariate analysis)

| | RFS | | | OS | | |
|---|---|---|---|---|---|---|
| No. | P | HR | 95% CI | P | HR | 95% CI |

GSE39582

| | | | | | | | |
|---|---|---|---|---|---|---|---|
| IL10RB^{high}/IL10RB^{low} | 157/312 | 0.9170 | 1.02 | 0.72 to 1.45 | 0.7690 | 0.95 | 0.67 to 1.35 |
| KRAS mut/KRAS WT | 169/279 | 0.0103 | 1.57 | 1.11 to 2.23 | 0.0533 | 1.40 | 1.00 to 1.96 |
| Within IL10RB^{low}: KRAS mut/KRAS WT | 112/187 | 0.8960 | 1.03 | 0.66 to 1.60 | 0.6640 | 1.10 | 0.72 to 1.67 |
| Within IL10RB^{high}: KRAS mut/KRAS WT | 57/92 | 0.0000 | 3.62 | 1.95 to 6.70 | 0.0039 | 2.43 | 1.33 to 4.45 |
| Within KRAS WT: IL10RB^{high}/IL10B^{low} | 92/187 | 0.0256 | 0.53 | 0.30 to 0.93 | 0.0938 | 0.65 | 0.40 to 1.08 |
| Within KRAS mut: IL10RB^{high}/IL10RB^{low} | 57/112 | 0.0196 | 1.84 | 1.10 to 3.07 | 0.2840 | 1.34 | 0.79 to 2.27 |

PETACC3

| | | | | | | | |
|---|---|---|---|---|---|---|---|
| IL10RB^{high}/IL10RB^{low} | 248/504 | 0.0131 | 1.36 | 1.07 to 1.74 | 0.2820 | 1.17 | 0.88 to 1.57 |
| KRAS mut/KRAS WT | 283/425 | 0.0215 | 1.34 | 1.04 to 1.72 | 0.0051 | 1.51 | 1.13 to 2.02 |
| Within IL10RB^{low}: KRAS mut/KRAS WT | 189/280 | 0.0856 | 1.33 | 0.96 to 1.84 | 0.0302 | 1.51 | 1.04 to 2.18 |
| Wilhin IL10RB^{high}: KRAS mut/KRAS WT | 94/145 | 0.0981 | 1.39 | 0.94 to 2.05 | 0.0577 | 1.57 | 0.99 to 2.51 |
| Within KRAS WT: IL10RB^{high}/IL10RB^{low} | 145/280 | 0.0520 | 1.40 | 1.00 to 1.96 | 0.3140 | 1.23 | 0.82 to 1.86 |
| Within KRAS mut: IL10RB^{high}/IL10RB^{low} | 94/189 | 0.0580 | 1.44 | 0.99 to 2.10 | 0.2920 | 1.26 | 0.82 to 1.94 |

Combined

| | | | | | | | |
|---|---|---|---|---|---|---|---|
| IL10RB^{high}/IL10RB^{low} | 590/1230 | 0.1190 | 1.15 | 0.96 to 1.37 | 0.4830 | 1.07 | 0.89 to 1.28 |
| KRAS mut/KRAS WT | 515/881 | 0.0006 | 1.43 | 1.16 to 1.75 | 0.0054 | 1.35 | 1.09 to 1.66 |
| Within IL10RB^{low}: KRAS mut/KRAS WT | 347/585 | 0.1250 | 1.22 | 0.95 to 1.58 | 0.1710 | 1.20 | 0.92 to 1.56 |
| Within IL10RB^{high}: KRAS mut/KRAS WT | 168/296 | 0.0002 | 1.89 | 1.36 to 2.63 | 0.0027 | 1.73 | 1.21 to 2.48 |
| Within KRAS WT: IL10RB^{high}/IL10RB^{low} | 296/585 | 0.7310 | 0.95 | 0.71 to 1.27 | 0.4440 | 0.89 | 0.66 to 1.20 |
| Within KRAS mut: IL10RB^{high}/IL10RB^{low} | 168/347 | 0.0172 | 1.45 | 1.07 to 1.97 | 0.1680 | 1.26 | 0.91 to 1.75 |

Table 3. KRAS mutation dramatically worsens prognosis in patients with IL10RB^{high} tumours. Univariate survival analysis of Stage II/III GSE39582 training set, PETACC3 and combined cohort validation sets. Effect of KRAS mutation status stratified according to IL10RB expression (high/low). Cox proportional Hazard analyses were performed on overall survival and relapse free survival. The hazard ratio, 95% confidence intervals, and associated Wald p-values are displayed. Significant results are highlighted in bold.
Abbreviations: RFS, relapse free survival; OS, overall survival; HR, hazard ratio; mut, mutant; .WT, wild type.

### KRAS Mutation is Prognostic in IL22RA1-High Patients in Proximal (Right-Sided) but not Distal (Left-Sided) CRC

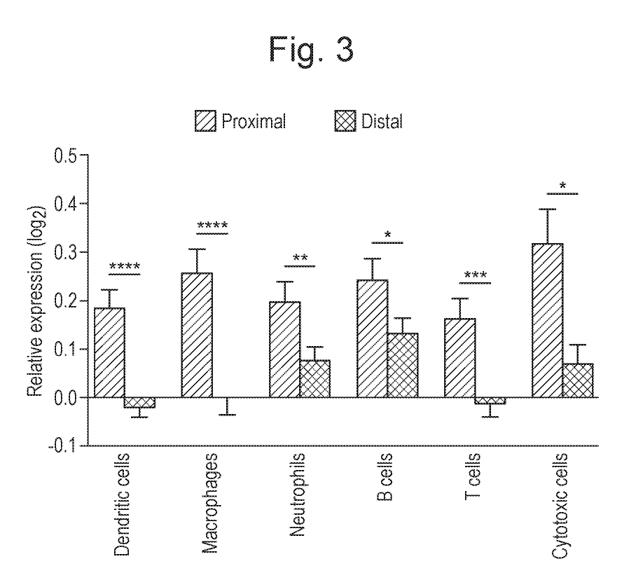

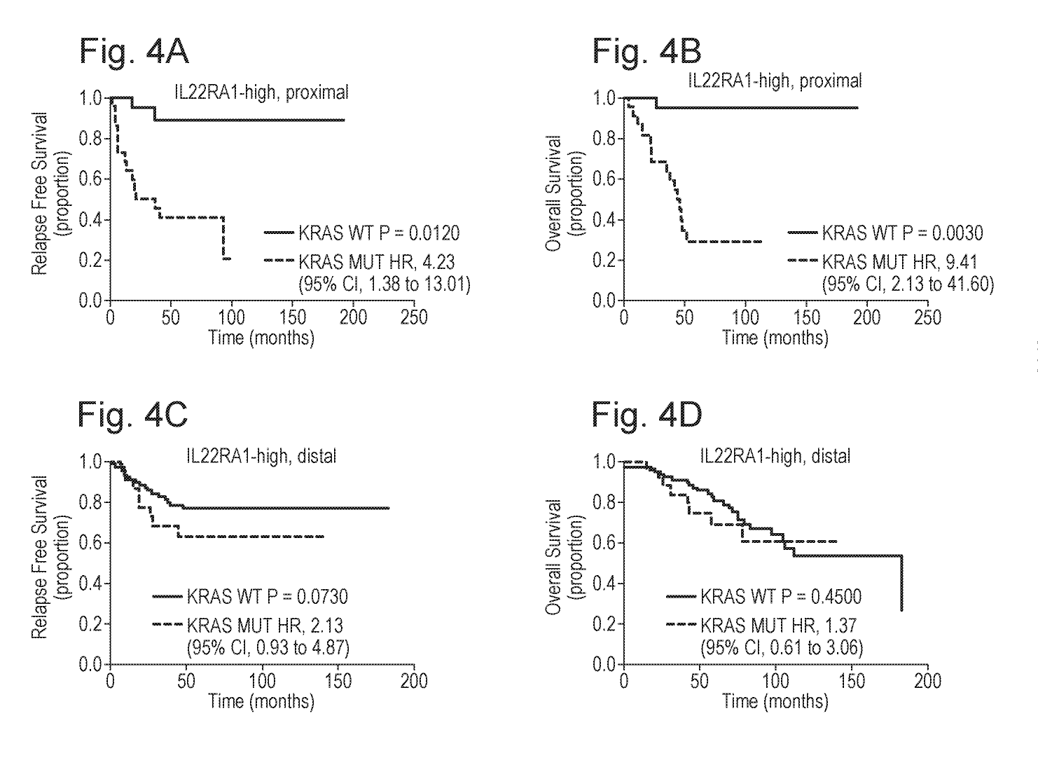

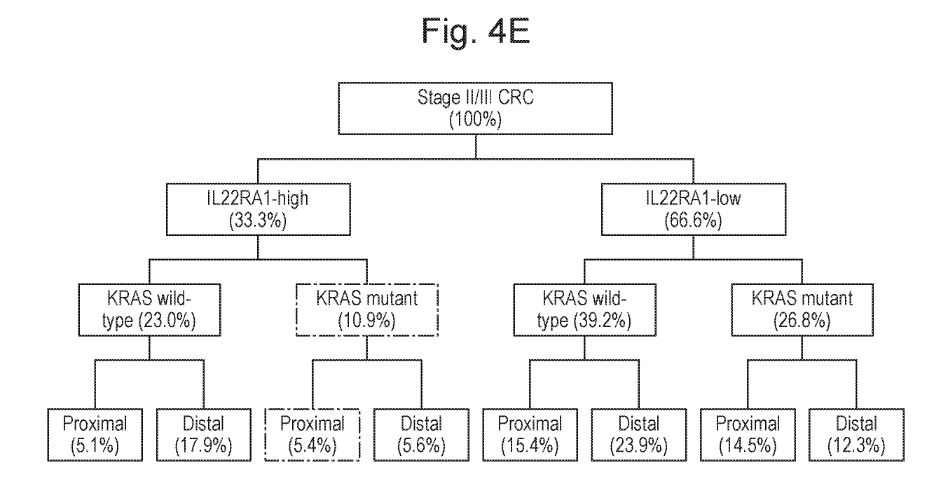

There are clear clinical and molecular differences between proximal and distal CRCs, deriving in part from the differing embryonic origin of the proximal and distal colon^{20,21}. Notably, proximal CRCs are more commonly associated with microsatellite instability and immune activation. Indeed, relative to distal tumors, proximal tumors in the GSE39582 dataset had significantly higher metagene scores for several leukocyte subsets including T cells, B cells, and antigen presenting cells (FIG. 3). Since IL-22 is produced by CD4⁺ T cells and innate lymphoid cells in the tumor microenvironment^{7,22}, we hypothesized that the IL22RA1-KRAS interaction may preferentially be prognostic in proximal CRCs. Indeed, when patients were first stratified based on tumor location (proximal vs distal), mutant KRAS dramatically worsened prognosis in patients with IL22RA1-high tumors specifically in the proximal colon (RFS, HR=4.23, 95% CI=1.38-13.01, P=0.012; OS, HR=9.41, 95% CI=2.13-41.60, P=0.003) (FIG. 4; Table 4). This observation was independent of microsatellite instability (MSI) and BRAF mutation, both of which are common features of proximal tumors (Table S3).

TABLE 4
Survival analysis (RFS and OS) tor Stage II, III patients of
GSE39582, PETACC3, and combined dataset (univariate analysis)

| | RFS | | | OS | | |
|---|---|---|---|---|---|---|
| No. | P | HR | 95% CI | P | HR | 95% CI |

GSE39582

| | | | | | | | | |
|---|---|---|---|---|---|---|---|---|
| IL22RA1^{low} | Proximal KRAS mut/KRAS WT | 65/69 | 0.414 | 0.73 | 0.35 to 1.54 | 0.871 | 0.95 | 0.50 to 1.80 |
| | Distal KRAS mut/KRAS WT | 55/107 | 0.031 | 1.77 | 1.05 to 2.97 | 0.531 | 1.20 | 0.67 to 2.15 |
| IL22RA1^{high} | Proximal KRAS mut/KRAS WT | 24/23 | 0.012 | 4.23 | 1.38 to 13.01 | 0.003 | 9.41 | 2.13 to 41.60 |
| | Distal KRAS mut/KRAS WT | 25/80 | 0.073 | 2.13 | 0.93 to 4.87 | 0.450 | 1.37 | 0.61 to 3.06 |

PETACC3

| | | | | | | | | |
|---|---|---|---|---|---|---|---|---|
| IL22RA1^{low} | Proximal KRAS mut/KRAS WT | 88/111 | 0.252 | 1.32 | 0.82 to 2.14 | 0.404 | 1.26 | 0.73 to 2.17 |
| | Distal KRAS mut/KRAS WT | 106/167 | 0.635 | 1.11 | 0.73 to 1.67 | 0.204 | 1.36 | 0.85 to 2.18 |
| IL22RA1^{high} | Proximal KRAS mut/KRAS WT | 26/39 | 0.008 | 2.84 | 1.31 to 6.12 | 0.033 | 2.52 | 1.08 to 5.90 |
| | Distal KRAS mut/KRAS WT | 63/108 | 0.281 | 1.32 | 0.79 to 2.21 | 0.088 | 1.73 | 0.92 to 3.25 |

Combined

| | | | | | | | | |
|---|---|---|---|---|---|---|---|---|
| IL22RA1^{low} | Proximal KRAS mut/KRAS WT | 187/264 | 0.672 | 1.12 | 0.75 to 1.68 | 0.705 | 0.93 | 0.63 to 1.36 |
| | Distal KRAS mut/KRAS WT | 173/305 | 0.129 | 1.28 | 0.93 to 1.77 | 0.222 | 1.25 | 0.87 to 1.79 |
| IL22RA1^{high} | Proximal KRAS mut/KRAS WT | 58/89 | 0.001 | 2.93 | 1.57 to 5.48 | 0.000 | 3.70 | 1.93 to 7.07 |
| | Distal KRAS mut/KRAS WT | 96/221 | 0.025 | 1.64 | 1.07 to 2.54 | 0.167 | 1.40 | 0.37 to 2.25 |

Table 4. KRAS mutation is prognostic in IL22RA1^{high} patients in proximal (right-sided) but not distal (left-sided) CRC. Univariate survival analysis of Stage II/III GSE39582 training set, PETACC3 and combined cohort validation sets. Effect of KRAS mutation status stratified according to tumor location (proximal/distal) and IL22RA1 expression level. Cox proportional Hazard analyses were performed on overall survival and relapse free survival. The hazard ratio, 95% confidence intervals, and associated Wald p-values are displayed. Significant results are highlighted in bold.
Abbreviations: RFS, relapse free survival; OS, overall survival; HR, hazard ratio; mut, mutant; WT, wild type.

TABLE S3
Survival analysis (RFS and OS) for PETACC3 patients comparing MSI versus MSS tumors (univariate analysis)

| | | | RFS | | | OS | | |
|---|---|---|---|---|---|---|---|---|
| PETACC3 | | No. | P | HR | 95% CI | P | HR | 95% CI |
| IL22RA1^{low} | Proximal MSI KRAS mut/KRAS WT | 13/38 | 0.164 | 0.23 | 0.03 to 1.82 | 0.473 | 0.46 | 0.06 to 3.85 |
| | Proximal MSS KRAS mut/KRAS WT | 72/68 | 0.144 | 1.50 | 0.87 to 2.63 | 0.552 | 1.20 | 0.66 to 2.17 |
| | Distal MSI KRAS mut/KRAS WT | 7/7 | 0.511 | 2.24 | 0.20 to 25.0 | 0.511 | 2.24 | 0.20 to 25.0 |
| | Distal MSS KRAS mut/KRAS WT | 90/148 | 0.455 | 1.18 | 0.76 to 1.82 | 0.154 | 1.44 | 0.87 to 2.38 |
| IL22RA1^{high} | Proximal MSI KRAS mut/KRAS WT | 2/6 | 0.008 | 2.84 | 1.32 to 6.25 | 0.033 | 2.52 | 1.08 to 5.88 |
| | Proximal MSS KRAS mut/KRAS WT | 22/32 | 0.003 | 3.49 | 1.54 to 7.69 | 0.017 | 3.07 | 1.22 to 7.69 |
| | Distal MSI KRAS mut/KRAS WT | 1/1 | N/A | N/A | N/A | N/A | N/A | N/A |
| | Distal MSS KRAS mut/KRAS WT | 62/104 | 0.281 | 1.32 | 0.79 to 2.22 | 0.087 | 1.73 | 0.93 to 3.23 |

Table S3. Right sided, KRAS mutant, IL22RA1^{high} patients have poor prognosis regardless of MSI status. Univariate survival analysis of PETACC3 cohort. Effect of KRAS mutation status stratified according to tumor location (proximal/distal), MSI status (MSS/MSI) and IL22RA1 expression level. Cox proportional Hazard analyses were performed on overall survival and relapse free survival. The hazard ratio, 95% confidence intervals, and associated Wald p-values are displayed. Significant results are highlighted in bold.
Abbreviations: RFS, relapse free survival; OS, overall survival; HR, hazard ratio; mut, mutant; WT, wild type; MSS, microsatellite stable; MSI, microsatellite instable.

### Discussion

Here, using the French cohort GSE39582 as a training set and the larger PETACC3 cohort as a validation set, we have identified an IL22RA1-high CRC subset in which KRAS mutation confers a strong negative prognosis. Furthermore, high expression of the second subunit of the heterodimeric IL-22 receptor, IL10RB, similarly distinguishes a patient subgroup in which KRAS mutation associates with poor outcome. The detrimental effect of having a KRAS mutation in a tumor that expresses high IL22RA1 preferentially affects proximal as compared to distal tumors. This association underscores a potential synergism between oncogenic KRAS and IL-22 signaling in colorectal cancer progression that requires further elucidation using detailed experimental approaches.

Cytokines do not induce neoplasia in the absence of oncogenic mutations. In murine models of IL-22 dependent CRC, the presence of existing oncogenic mutations or treatment with a mutagenic agent was required for carcinogensis.^{4,5} However, the specific driver mutations in these murine models were not characterized. Here we have demonstrated that a link between IL22RA1 expression and poor patient outcome is specifically dependent on the presence of KRAS mutations. Neither TP53 nor BRAF interacted with IL22RA1 in this manner (data not shown).

It has previously been demonstrated through prospective analysis of the PETACC-3 cohort that KRAS mutation status alone has no major prognostic value for RFS or OS in stage II and III CRC patients who receive adjuvant chemotherapy.¹⁷ This is in accordance with a number of smaller retrospective studies.^{23,24} Therefore, the negative prognostic effect of KRAS mutation in patients with IL22RA1-high tumors may be due to a previously unrecognized synergy between IL-22 signaling and a constitutively active Ras pathway. To the best of our knowledge, this synergy is unique to IL-22. Despite the extensively described tumor-promoting role of IL-6 (which is biochemically similar to IL-22), IL6R does not interact with KRAS. It was recently found that oncogenic Kras promotes IL-17 signaling in a pre-invasive pancreatic neoplasia (PanIN) murine model and that oncogenic Kras can drive expression of the IL-17 receptor.²⁵ STAT3, a major mediator of IL-22 and IL-6 signal transduction, is important for Kras-dependent PanIN formation.²⁶ The potential synergism between oncogenic mutations and inflammatory cytokine signaling has not, however, been studied extensively in colorectal cancer. To our knowledge, this is the first reported evidence of an IL22RA1-KRAS synergy in CRC.

The prognostic value of KRAS in IL22RA1-high tumors is limited to proximal disease. From an immunological perspective, this is logical given that proximal tumors tend to be associated with immune activation.²⁰ CD4⁺ T cells and innate lymphoid cells secrete IL-22 in both homeostasis and pathology downstream of microbial stimuli. Interestingly, it was recently reported that bacterial biofilms are almost universally present on proximal but not distal CRCs in independent American and Malaysian cohorts.²⁷ It is conceivable that enhanced IL-22 signaling may occur in the proximal versus distal colon due to differences in the composition and structure of the intestinal microbiota.

Proximal CRCs frequently display MSI, which is thought to enhance tumor immunogenicity and is associated with elevated immune activity and favorable prognosis.^{28,29} Furthermore, recent data suggest that this subset may be an attractive target for checkpoint blockade immunotherapy.³⁰ MSI tumors are also commonly BRAF mutants, and this molecular subtype of disease, namely CMS1, has the best relapse free survival of the four consensus CRC molecular subtypes.³¹ BRAF and KRAS mutations are known to be mutually exclusive, making it possible that the poor prognosis of proximal, IL22RA1-high, KRAS mutant patients was an epiphenomenon of the good prognosis of the proximal MSI tumors. This was not the case however, as when the proximal MSI tumors were excluded from the analysis, the prognostic significance of KRAS mutation in right-sided IL22RA1-high patients was sustained (Table S3). A similar analysis focused only on MSI tumors was not possible due to limitations of sample size. Two recent studies in large CRC cohorts (n=2080³², n=2720³³) have demonstrated that in mismatch repair (MMR)-proficient CRCs treated with standard chemotherapy, KRAS mutants (approximately 35% of patients) have increased CRC specific mortality.^{32,33} Because this group tends to be resistant to anti-EGFR therapy they are relatively bereft of alternative therapeutic options. The proximal, IL22RA1-high subgroup of MMR-proficient KRAS-mutants could thus represent a population in which anti-IL-22 immunomodulatory therapy may be beneficial. Notably, at least one anti-IL-22 monoclonal antibody (Fezakinumab, Pfizer) has progressed to phase II clinical trials for inflammatory conditions.

One of the commonly cited limitations of CRC biomarker studies is that most have been conducted in patients who received 5-FU and not the current standard of care, FOLFOX. A related caveat of our discovery cohort (GSE39582) was treatment heterogeneity. However, the KRAS-IL22RA1 interaction was clearly evident in the larger and more homogenous PETACC3 dataset in which all patients received the current standard of care, suggesting independence from therapeutic status.

Based on the evidence presented here, we would propose a stratification strategy in which proximal CRCs, which are already subject to routine KRAS mutation typing, are additionally typed for IL22RA1 expression by mRNA analysis, most likely through a quantitative PCR-based approach. Patients with high intratumoral IL22RA1 expression and KRAS mutation would be predicted to have a lower likelihood of response to conventional chemotherapy and poor survival outcomes, suggesting that closer monitoring and more aggressive or alternative therapeutic strategies could be beneficial. Although limited alternative therapies exist for such patients, blockade of IL-22 in combination with standard therapy is an intriguing possibility. Notably, although the overall incidence of CRC has declined in recent years, the incidence of proximal CRCs continues to rise, highlighting the need to improve clinical management of these tumors.³⁴

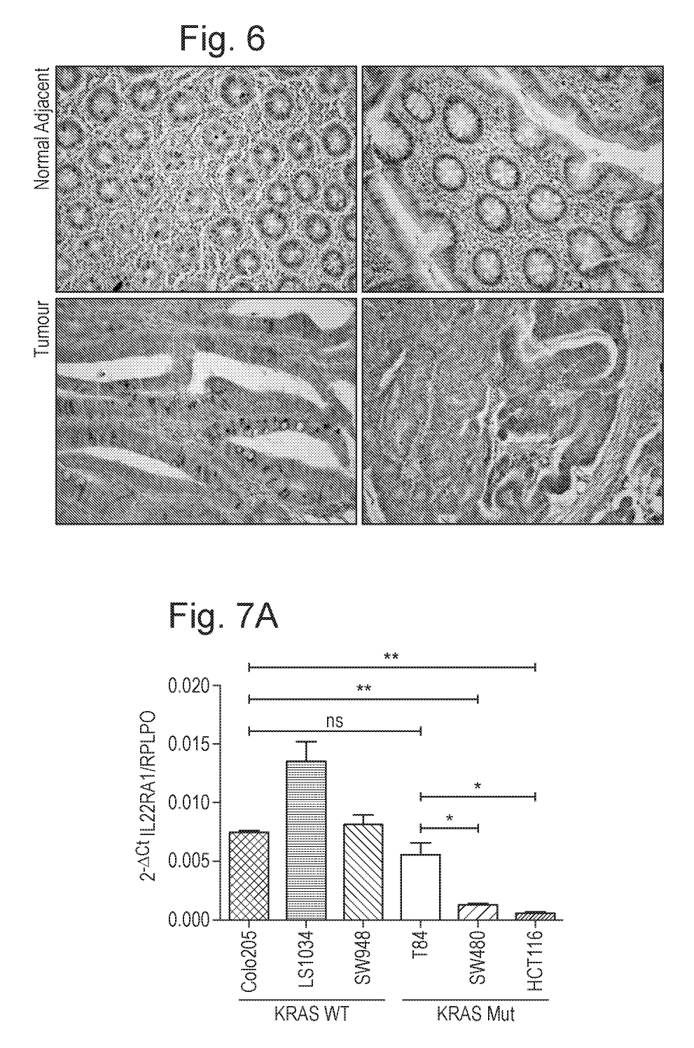

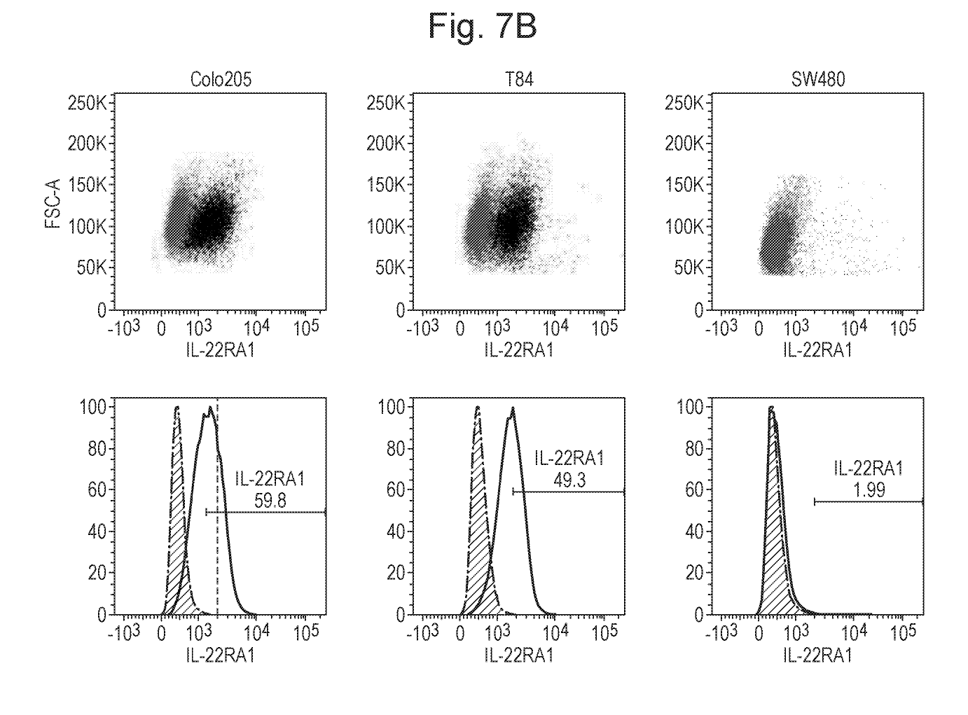

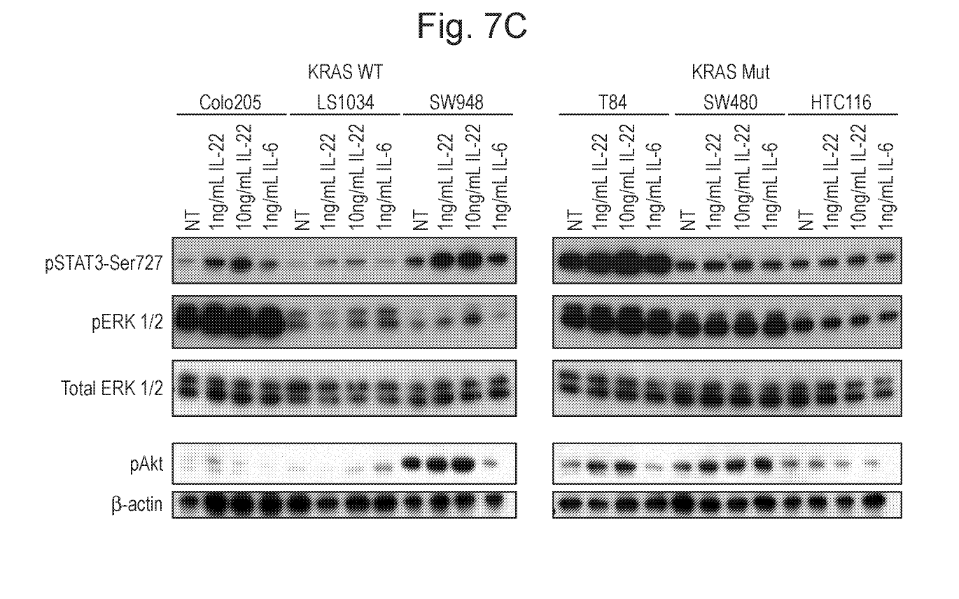

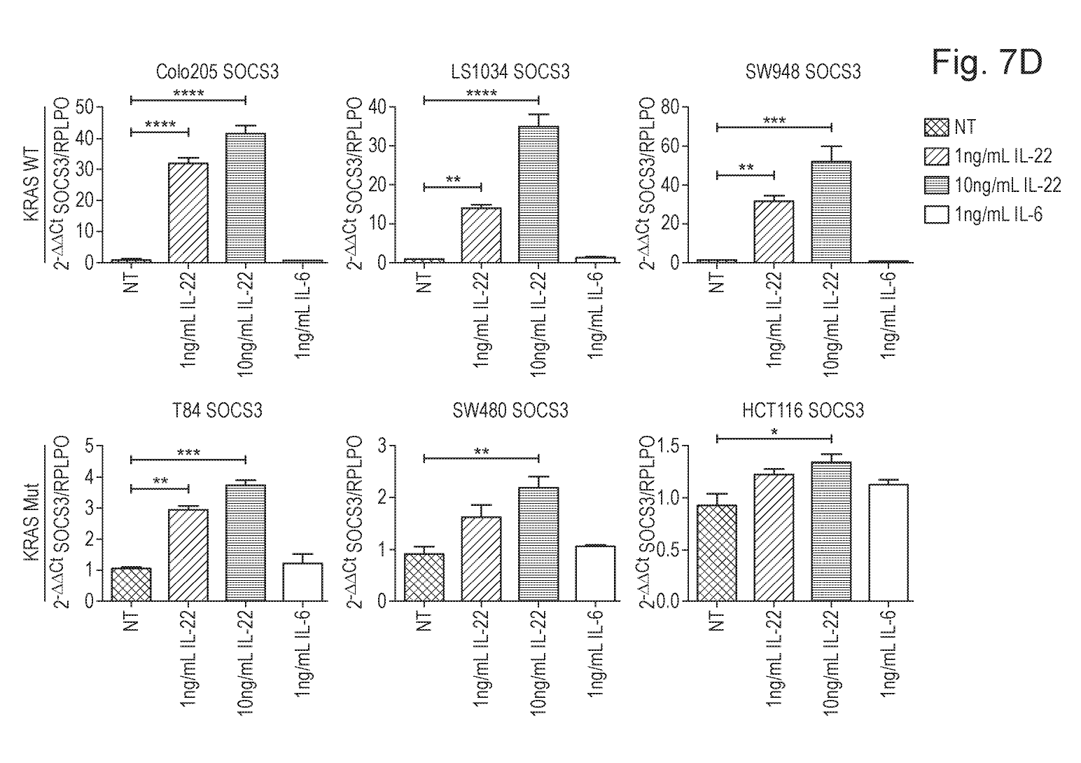

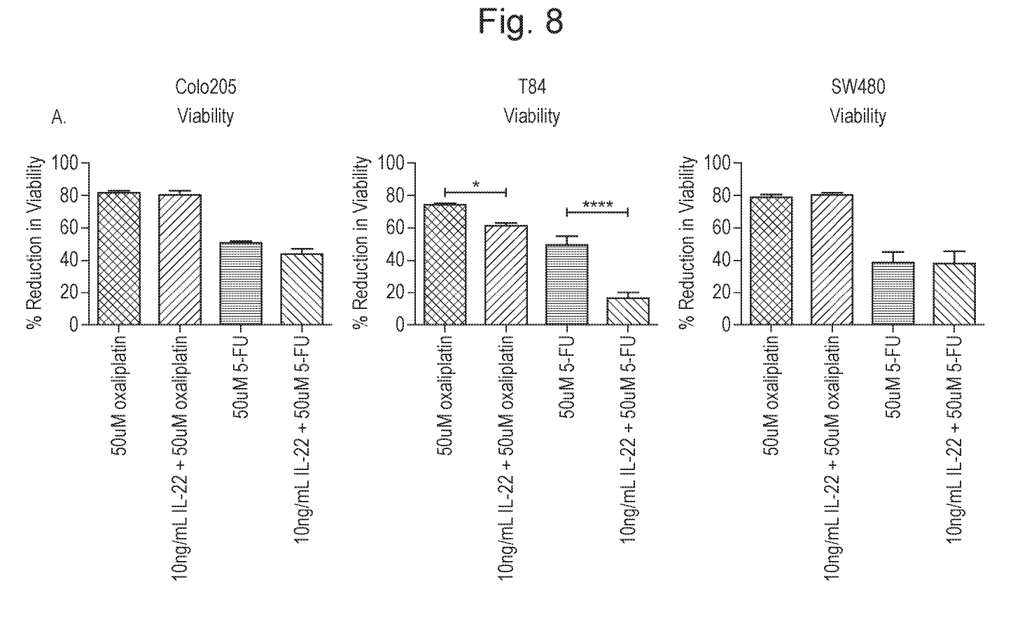

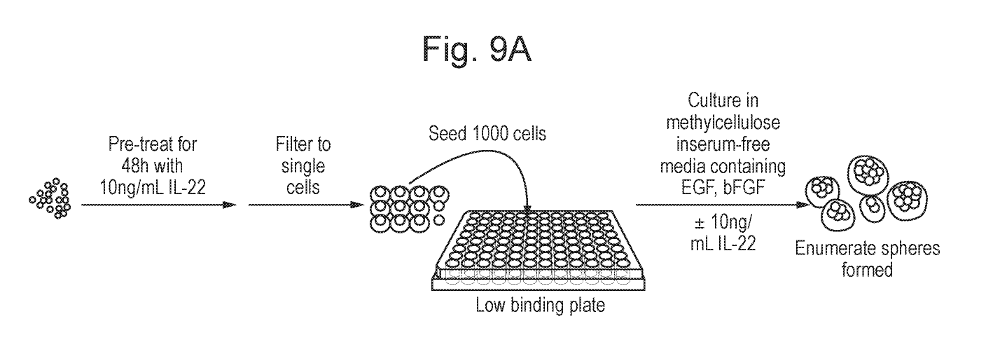

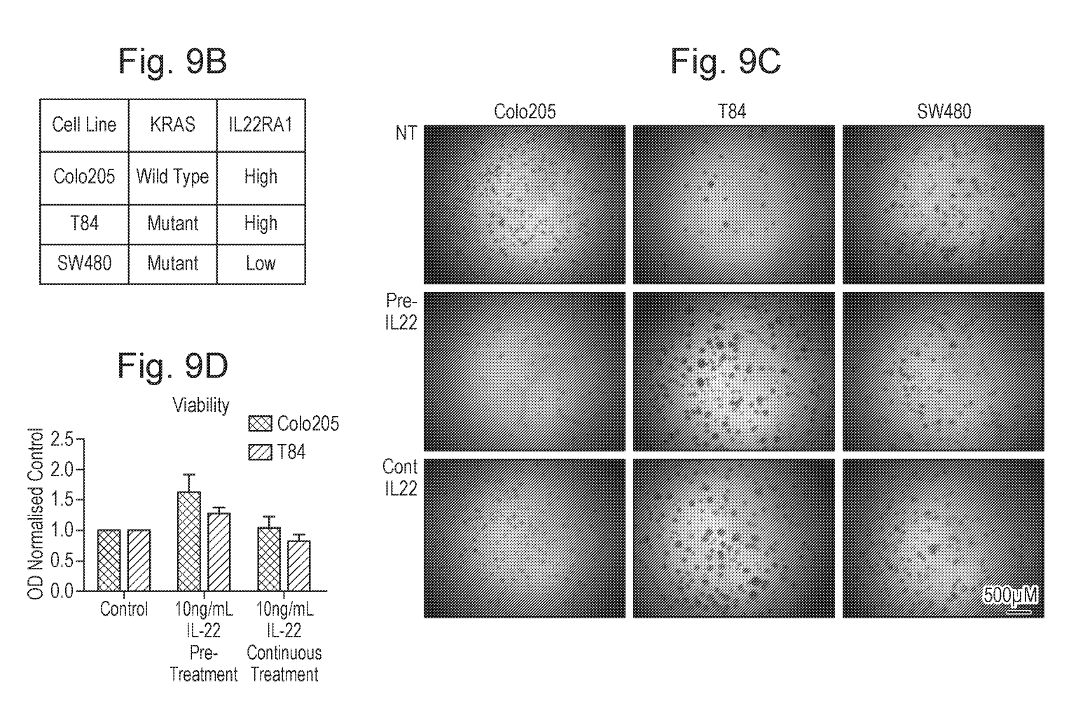

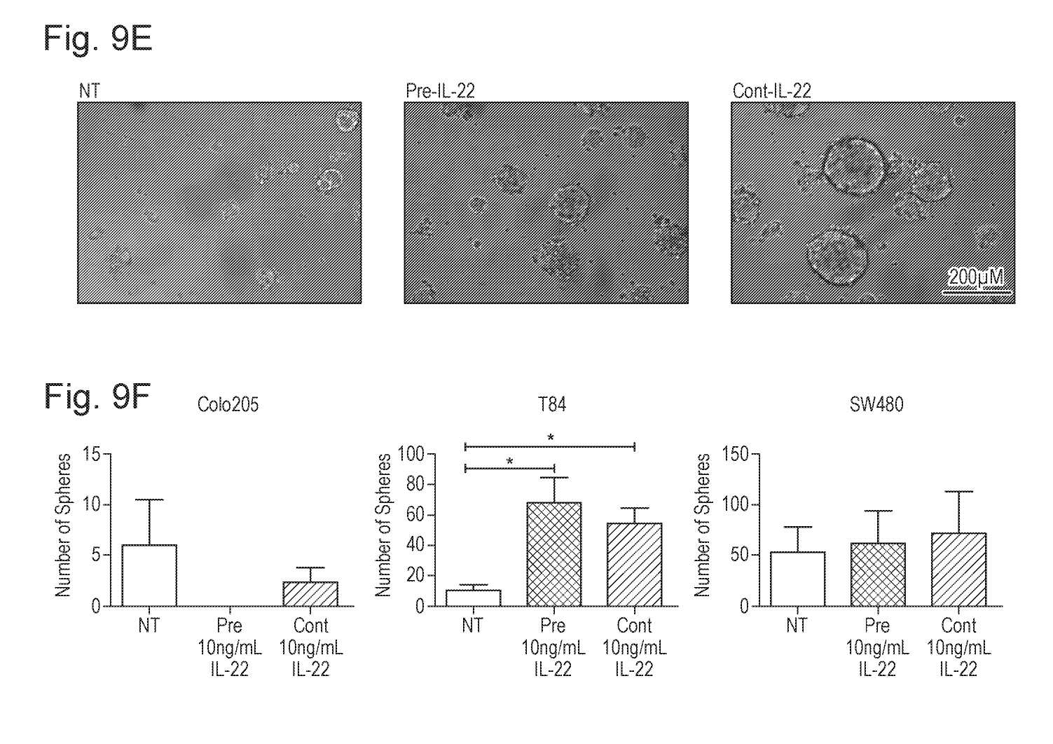

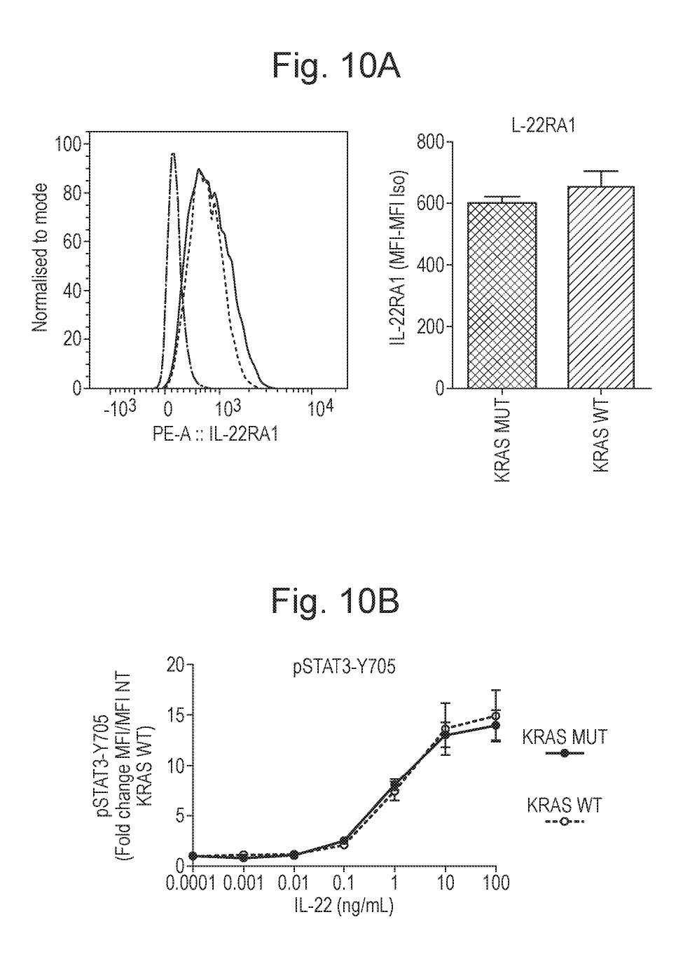

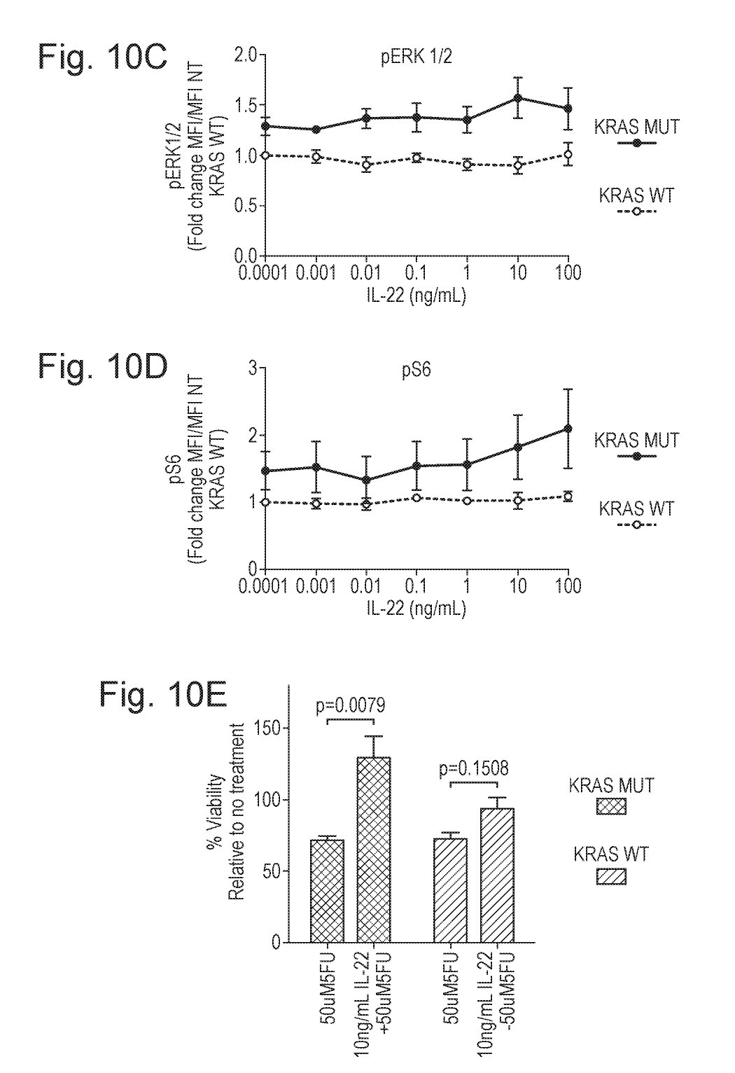

Because IL22RA1 expression manifests as a continuous, non-biphasic variable, further prospective studies assessing IL22RA1 expression are required to characterize a clinically relevant cut-point (FIG. 5). We have also shown that IL-22R protein is detectable in human FFPE tissue sections (FIG. 6), raising the possibility of developing a standardized immunohistochemical assay. Although IL10RB also interacts with KRAS, its expression is more promiscuous. While IL22RA1 expression is restricted to the tumor epithelium (FIG. 6), IL10RB is expressed by most intestinal cell types, which complicates the interpretation of the signal. Two recent studies have demonstrated that the gene expression patterns which delineate the consensus CRC molecular subtypes are highly influenced by the tumor stroma.^{35,36} The epithelial restriction of IL22RA1 may thus make it a more reliable and biologically interpretable marker.

In conclusion, we have identified a proximal, IL22RA1-high, KRAS mutant CRC molecular subtype with dramatically worsened prognosis relative to KRAS wild type or IL22RA1-low counterparts. To our knowledge, this is the first time that cytokine receptor expression has been examined in the context of oncogenic mutations in clinical transcriptomic data. Our data provide additional justification for the assessment of KRAS mutations in CRC patients, which has until now been clinically beneficial only for prediction of cetuximab responsiveness. Further clinical investigation of IL-22 and the IL22RA1-KRAS interaction in CRC is warranted, and basic studies will be required to elucidate the functional nature of this apparent IL-22/KRAS synergy.

### REFERENCES

1. Jess, T., Rungoe, C. & Peyrin-Biroulet, L. Risk of colorectal cancer in patients with ulcerative colitis: a meta-analysis of population-based cohort studies. Clin. Gastroenterol. Hepatol. 10, 639-45 (2012).
2. Hanahan, D. & Weinberg, R. a. Hallmarks of cancer: the next generation. Cell 144, 646-74 (2011).
3. Elinav, E. et al. Inflammation-induced cancer: crosstalk between tumours, immune cells and microorganisms. Nat. Rev. Cancer 13, 759-71 (2013).
4. Kirchberger, S. et al. Innate lymphoid cells sustain colon cancer through production of interleukin-22 in a mouse model. J. Exp. Med. 210, 917-931 (2013).
5. Huber, S. et al. IL-22BP is regulated by the inflammasome and modulates tumorigenesis in the intestine. Nature 491, 259-63 (2012).
6. Zhuang, Y. et al. Increased intratumoral IL-22-producing CD4(+) T cells and Th22 cells correlate with gastric cancer progression and predict poor patient survival. Cancer Immunol. Immunother. 61, 1965-75 (2012).
7. Kryczek, I. et al. IL-22(+)CD4(+) T cells promote colorectal cancer stemness via STAT3 transcription factor activation and induction of the methyltransferase DOT1L. Immunity 40, 772-784 (2014).
8. Xie, M. H. et al. Interleukin (IL)-22, a novel human cytokine that signals through the interferon receptor-related proteins CRF2-4 and IL-22R. J. Biol. Chem. 275, 31335-9 (2000).
9. Gurney, A. L. IL-22, a Th1 cytokine that targets the pancreas and select other peripheral tissues. Int. Immunopharmacol. 4, 669-77 (2004).
10. Mitra, A., Raychaudhuri, S. K. & Raychaudhuri, S. P. IL-22 induced cell proliferation is regulated by PI3K/Akt/mTOR signaling cascade. Cytokine 60, 38-42 (2012).
11. Xu, M.-J et al. IL-22 ameliorates renal ischemia-reperfusion injury by targeting proximal tubule epithelium. J. Am. Soc. Nephrol. 25, 967-77 (2014).
12. Lim, C. & Savan, R. The role of the IL-22/IL-22R1 axis in cancer. Cytokine Growth Factor Rev. 25, 257-271 (2014).
13. Karapetis, C. et al. K-ras Mutations and Benefit from Cetuximab in Advanced Colorectal Cancer. N. Engl. J. Med. 359, 1757-1765 (2008).
14. Bokemeyer, C. & Bondarenko, I. Efficacy according to biomarker status of cetuximab plus FOLFOX-4 as first-line treatment for metastatic colorectal cancer: the OPUS study. Ann. Oncol. 22, 1535-46 (2011).
15. Van Cutsem, E. et al. Fluorouracil, Leucovorin, and Irinotecan Plus Cetuximab Treatment and RAS Mutations in Colorectal Cancer. J. Clin. Oncol. (2015). doi:10.1200/JCO.2014.59.4812
16. Marisa, L. et al. Gene expression classification of colon cancer into molecular subtypes: characterization, validation, and prognostic value. PLoS Med. 10, e1001453 (2013).
17. Roth, A. D. et al. Prognostic role of KRAS and BRAF in stage II and III resected colon cancer: results of the translational study on the PETACC-3, EORTC 40993, SAKK 60-00 trial. J. Clin. Oncol. 28, 466-74 (2010).
18. Bollrath, J. et al. gp130-mediated Stat3 activation in enterocytes regulates cell survival and cell-cycle progression during colitis-associated tumorigenesis. Cancer Cell 15, 91-102 (2009).
19. Grivennikov, S. et al. IL-6 and Stat3 are required for survival of intestinal epithelial cells and development of colitis-associated cancer. Cancer Cell 15, 103-13 (2009).
20. Missiaglia, E. et al. Distal and proximal colon cancers differ in terms of molecular, pathological, and clinical features. Ann. Oncol. 25, 1995-2001 (2014).
21. Maus, M. K. H. et al. Distinct gene expression profiles of proximal and distal colorectal cancer: implications for cytotoxic and targeted therapy. Pharmacogenomics J. 1-9 (2014). doi:10.1038/tpj.2014.73
22. Kirchberger, S. et al. Innate lymphoid cells sustain colon cancer through production of interleukin-22 in a mouse model. J. Exp. Med. 210, 917-931 (2013).
23. González-Aguilera, J. J., Oliart, S., Azcoita, M. M. & Fernandez-Peralta, A. M. Simultaneous Mutations in K-ras and TP53 Are Indicative of Poor Prognosis in Sporadic Colorectal Cancer. Am. J. Clin. Oncol. 27, (2004).
24. Westra, J. L. et al. Determination of TP53 mutation is more relevant than microsatellite instability status for the prediction of disease-free survival in adjuvant-treated stage III colon cancer patients. J. Clin. Oncol. 23, 5635-5643 (2005).
25. McAllister, F. et al. Oncogenic Kras Activates a Hematopoietic-to-Epithelial IL-17 Signaling Axis in Preinvasive Pancreatic Neoplasia. Cancer Cell 25, 621-637 (2014).
26. Corcoran, R. B. et al. STAT3 plays a critical role in KRAS-induced pancreatic tumorigenesis. Cancer Res. 71, 5020-9 (2011).
27. Dejea, C. M. et al. Microbiota organization is a distinct feature of proximal colorectal cancers. Proc. Natl. Acad. Sci. U.S.A. 1-6 (2014). doi:10.1073/pnas.1406199111
28. Dolcetti, R. et al. High prevalence of activated intraepithelial cytotoxic T lymphocytes and increased neoplastic cell apoptosis in colorectal carcinomas with microsatellite instability. Am. J. Pathol. 154, 1805-1813 (1999).
29. Guidoboni, M. et al. Microsatellite instability and high content of activated cytotoxic lymphocytes identify colon cancer patients with a favorable prognosis. Am. J. Pathol. 159, 297-304 (2001).
30. Llosa, N. J. et al. The Vigorous Immune Microenvironment of Microsatellite Instable Colon Cancer Is Balanced by Multiple Counter-Inhibitory Checkpoints. Cancer Discov. 43-52 (2014). doi:10.1158/2159-8290.CD-14-0863
31. Dienstmann, R. et al. O-0025 * Colorectal Cancer Subtyping Consortium (Crcsc) Identifies Consensus of Molecular Subtypes. Ann. Oncol. 25, ii115-ii115 (2014).
32. Phipps, A. I. et al. Association Between Molecular Subtypes of Colorectal Cancer and Patient Survival. Gastroenterology 148, 77-87.e2 (2015).
33. Sinicrope, F. a et al. Molecular Markers Identify Subtypes of Stage III Colon Cancer Associated With Patient Outcomes. Gastroenterology 148, 88-99 (2014).
34. Cheng, L., Eng, C., Nieman, L. Z., Kapadia, A. S. & Du, X. L. Trends in Colorectal Cancer Incidence by Anatomic Site and Disease Stage in the United States From 1976 to 2005. Am. J. Clin. Oncol. 34, (2011).
35. Isella, C. et al. Stromal contribution to the colorectal cancer transcriptome. Nat. Genet. 1-11 (2015). doi:10.1038/ng.3224
36. Calon, A. et al. Stromal gene expression defines poor-prognosis subtypes in colorectal cancer. Nat. Genet. (2015). doi:10.1038/ng.3225
37. Lim, C. & Sayan, R. The role of the IL-22/IL-22R1 axis in cancer. Cytokine Growth Factor Rev. 25, 257-271 (2014).
38. Dumoutier, L., de Meester, C., Tavernier, J. & Renauld, J.-C. New activation modus of STAT3: a tyrosine-less region of the interleukin-22 receptor recruits STAT3 by interacting with its coiled-coil domain. J. Biol. Chem. 284, 26377-84 (2009).
39. Yoshimura, A. & Yasukawa, H. JAK's SOCS: a mechanism of inhibition. Immunity 36, 157-9 (2012).
40. Mitra, A., Raychaudhuri, S. K. & Raychaudhuri, S. P. IL-22 induced cell proliferation is regulated by PI3K/Akt/mTOR signaling cascade. Cytokine 60, 38-42 (2012).
41. Xu, M.-J. et al. IL-22 ameliorates renal ischemia-reperfusion injury by targeting proximal tubule epithelium. J. Am. Soc. Nephrol. 25, 967-77 (2014).
42. Xu, W. et al. A soluble class II cytokine receptor, IL-22RA2, is a naturally occurring IL-22 antagonist. Proc. Natl. Acad. Sci. U.S.A. 98, 9511-6 (2001).

1. A method of treating in a patient colorectal cancer which comprises a KRAS mutation and a high amount of interleukin 22 (IL-22) receptor, the method comprising administering to the patient an inhibitor of IL-22 signalling and thereby treating the cancer.

2. A method according to claim 1, wherein the method is for treating colorectal cancer in a patient that has been selected for treatment on the basis that the cancer comprises a KRAS mutation and a high amount of IL-22 receptor.

3. A method according to claim 1, wherein the cancer is proximal colorectal cancer.

4. A method according to claim 3, wherein the method is for treating colorectal cancer in a patient that has been selected for treatment on the basis that the cancer is proximal colorectal cancer which comprises a KRAS mutation and a high amount of IL-22 receptor.

5. A method according to claim 1, wherein the cancer comprises (i) a variant of the sequence shown in SEQ ID NO:1 or 2 which comprises one or more point mutations or (ii) a polynucleotide which encodes the variant in (i).

6. A method according to claim 5, wherein the variant comprises a point mutation at one or more of positions 12, 13, 14, 59, 61, 117, 120, 144, 145 and 146 of SEQ ID NO:1 or 2.

7. A method according to claim 5, wherein the variant comprises one or more of the following point mutations (a) G12A, G12C, G12D, G12R, G12S or G12V, (b) G13A, G13C, G13D, G13R or G13V, (c) V14I, (d) A59G, (e) Q61H, Q61K, Q61L or Q61R, (f) K117N, (g) L120V, (h) S145T and (i) A146P, A146T and A146V.

8. A method according to claim 1, wherein the cancer comprises a high amount of IL-22 receptor relative to other cancers of the same type.

9. A method according to claim 8, wherein the cancer comprises an amount of IL-22 receptor which is greater than the 60^{th} or 67^{th} percentile of amount in a cohort of colorectal cancers.

10. A method according to claim 1, wherein the cancer comprises a high amount of IL-22 receptor subunit alpha-1 (IL-22RA1).

11. A method according to claim 10, wherein the cancer comprises a high amount of IL-22RA1 protein and/or a high amount of IL22RA1 mRNA.

12. A method according to claim 11, wherein the IL-22RA1 protein comprises the sequence shown in SEQ ID NO:3 or a variant thereof and/or the IL22RA1 mRNA comprises the sequence shown in SEQ ID NO:4 or a variant thereof.

13. A method according to claim 1, wherein the inhibitor is an inhibitor of the IL-22 receptor or IL-22RA1.

14. A method according to claim 1, wherein the inhibitor is an inhibitor of IL-22, interleukin 20 (IL-20) or interleukin (IL-24).

15. A method according to claim 1, wherein the inhibitor is a small molecule inhibitor, a protein, an antibody, a polynucleotide, an oligonucleotide, an antisense RNA, a small interfering RNA (siRNA) or a small hairpin RNA (shRNA).

16. A method according to claim 1, wherein the inhibitor is administered in combination with another cancer therapy.

17. A method according to claim 1, wherein the patient is human.

18. A method of treating colorectal cancer in a patient, the method comprising (a) determining whether or not the cancer comprises a KRAS mutation and measuring the amount of IL-22 receptor in the cancer and (b), if the cancer comprises a KRAS mutation and a high amount of IL-22 receptor, administering to the patient an inhibitor of IL-22 signalling and thereby treating the cancer.

19. An inhibitor of IL-22 signalling for use in a method of treating in a patient colorectal cancer which comprises a KRAS mutation and a high amount of IL-22 receptor.

20. Use of an inhibitor of IL-22 signalling in the manufacture of a medicament for treating in a patient colorectal cancer which comprises a KRAS mutation and a high amount of IL-22 receptor.

21. A kit for treating colorectal cancer comprising (a) means for testing whether or not the cancer comprises a KRAS mutation and for measuring the amount of IL-22 receptor and (b) an inhibitor of IL-22 signalling.

22. A method for prognosing colorectal cancer in a patient, the method comprising determining whether or not the cancer comprises a KRAS mutation and measuring the amount of IL-22 receptor in the cancer, wherein the presence of a KRAS mutation and a high amount of IL-22 receptor in the cancer indicates that the patient has a worse prognosis than in the absence of a KRAS mutation and/or in the presence of a low amount of IL-22 receptor.

23. A method for determining whether or not a patient with colorectal cancer is likely to respond to therapy with an inhibitor of IL-22 signalling, the method comprising determining whether or not the cancer comprises a KRAS mutation and measuring the amount of IL-22 receptor in the cancer, wherein the presence of a KRAS mutation and a high amount of IL-22 receptor in the cancer indicates that the patient is likely to respond to therapy with an inhibitor of IL-22 signalling.

24. An in vitro assay for determining whether or not a patient with colorectal cancer is likely to respond to therapy with an inhibitor of IL-22 signalling, the assay comprising determining whether or not a sample from the cancer comprises a KRAS mutation and measuring the amount of IL-22 receptor in the cancer, wherein the presence of a KRAS mutation and a high amount of IL-22 receptor in the sample indicates that the patient is likely to respond to therapy with an inhibitor of IL-22 signalling.

25. An in vitro assay for prognosing colorectal cancer in a patient, the assay comprising determining whether or not a sample from the cancer comprises a KRAS mutation and measuring the amount of IL-22 receptor in the cancer, wherein the presence of a KRAS mutation and a high amount of IL-22 receptor in the cancer indicates that the patient has a worse prognosis than in the absence of a KRAS mutation and/or in the presence of a low amount of IL-22 receptor.

26. A system for determining whether or not a patient with colorectal cancer is likely to respond to therapy with an inhibitor of IL-22 signalling, the system comprising
(a) a measuring module for determining whether or not the cancer comprises a KRAS mutation and for measuring the amount of IL-22 receptor in the cancer,
(b) a storage module configured to store control data and output data from the measuring module,
(c) a computation module configured to provide a comparison between the value of the output data from the measuring module and the control data; and
(d) an output module configured to display whether or not the patient is likely to respond to therapy with an inhibitor of IL-22 signalling based on the comparison,
wherein the presence of a KRAS mutation and a high amount of IL-22 receptor in the cancer indicates that the patient is likely to respond to therapy with an inhibitor of IL-22 signalling.

27. A system for prognosing colorectal cancer in a patient, the system comprising
(a) a measuring module for determining whether or not the cancer comprises a KRAS mutation and for measuring the amount of IL-22 receptor in the cancer,
(b) a storage module configured to store control data and output data from the measuring module,
(c) a computation module configured to provide a comparison between the value of the output data from the measuring module and the control data; and
(d) an output module configured to display the patient's prognosis,
wherein the presence of a KRAS mutation and a high amount of IL-22 receptor in the cancer indicates that the patient has a worse prognosis than in the absence of a KRAS mutation or in the presence of a low amount of IL-22 receptor.


SEQUENCE LISTING (11 sequences)

SEQ ID NO: 1 - protein, 189 aa, Homo sapiens [sequence omitted]

SEQ ID NO: 2 - protein, 188 aa, Homo sapiens [sequence omitted]

SEQ ID NO: 3 - protein, 574 aa, Homo sapiens
MRTLLTILTVGSLAAHAPEDPSDLLQHVKFQSSNFENILTWDSGPEGTPDTVYSIEYKTYGERDWVAKKGCQRITRKSCNLTVETGNLTELYYARVTAVSAGGRSATKMTDRFSSLQHTTLKPPDVTCISKVRSIQMIVHPTPTPIRAGDGHRLTLEDIFHDLFYHLELQVNRTYQMHLGGKQREYEFFGLTPDTEFLGTIMICVPTWAKESAPYMCRVKTLPDRTWTYSFSGAFLFSMGFLVAVLCYLSYRYVTKPPAPPNSLNVQRVLTFQPLRFIQEHVLIPVFDLSGPSSLAQPVQYSQIRVSGPREPAGAPQRHSLSEITYLGQPDISILQPSNVPPPQILSPLSYAPNAAPEVGPPSYAPQVTPEAQFPFYAPQAISKVQPSSYAPQATPDSWPPSYGVCMEGSGKDSPTGTLSSPKHLRPKGQLQKEPPAGSCMLGGLSLQEVTSLAMEESQEAKSLHQPLGICTDRTSDPNVLHSGEEGTPQYLKGQLPLLSSVQIEGHPMSLPLQPPSRPCSPSDQGPSPWGLLESLVCPKDEAKSPAPETSDLEQPTELDSLFRGLALTVQWES

SEQ ID NO: 4 - DNA, 2988 bp, Homo sapiens
agatcccgcccagaacctggaattccagagcacacagggccaggacacctttgtacccctgctccccaccggcacaggtcccacccacccccttctccccgccccatgcccgggtggagccagctgccagggcgccaagactcccagccctctctctgtgcacggcagagtagaggccaagggagggctctgtgccagccccgatgaggacgctgctgaccatcttgactgtgggatccctggctgctcacgcccctgaggacccctcggatctgctccagcacgtgaaattccagtccagcaactttgaaaacatcctgacgtgggacagcgggccggagggcaccccagacacggtctacagcatcgagtataagacgtacggagagagggactgggtggcaaagaagggctgtcagcggatcacccggaagtcctgcaacctgacggtggagacgggcaacctcacggagctctactatgccagggtcaccgctgtcagtgcgggaggccggtcagccaccaagatgactgacaggttcagctctctgcagcacactaccctcaagccacctgatgtgacctgtatctccaaagtgagatcgattcagatgattgttcatcctacccccacgccaatccgtgcaggcgatggccaccggctaaccctggaagacatcttccatgacctgttctaccacttagagctccaggtcaaccgcacctaccaaatgcaccttggagggaagcagagagaatatgagttcttcggcctgacccctgacacagagttccttggcaccatcatgatttgcgttcccacctgggccaaggagagtgccccctacatgtgccgagtgaagacactgccagaccggacatggacctactccttctccggagccttcctgttctccatgggcttcctcgtcgcagtactctgctacctgagctacagatatgtcaccaagccgcctgcacctcccaactccctgaacgtccagcgagtcctgactttccagccgctgcgcttcatccaggagcacgtcctgatccctgtctttgacctcagcggccccagcagtctggcccagcctgtccagtactcccagatcagggtgtctggacccagggagcccgcaggagctccacagcggcatagcctgtccgagatcacctacttagggcagccagacatctccatcctccagccctccaacgtgccacctccccagatcctctccccactgtcctatgccccaaacgctgcccctgaggtcgggcccccatcctatgcacctcaggtgacccccgaagctcaattcccattctacgccccacaggccatctctaaggtccagccttcctcctatgcccctcaagccactccggacagctggcctccctcctatggggtatgcatggaaggttctggcaaagactcccccactgggacactttctagtcctaaacaccttaggcctaaaggtcagcttcagaaagagccaccagctggaagctgcatgttaggtggcctttctctgcaggaggtgacctccttggctatggaggaatcccaagaagcaaaatcattgcaccagcccctggggatttgcacagacagaacatctgacccaaatgtgctacacagtggggaggaagggacaccacagtacctaaagggccagctccccctcctctcctcagtccagatcgagggccaccccatgtccctccctttgcaacctccttcccgtccatgttccccctcggaccaaggtccaagtccctggggcctgctggagtcccttgtgtgtcccaaggatgaagccaagagcccagcccctgagacctcagacctggagcagcccacagaactggattctcttttcagaggcctggccctgactgtgcagtgggagtcctgaggggaatgggaaaggcttggtgcttcctccctgtccctacccagtgtcacatccttggctgtcaatcccatgcctgcccacgccacacactctgcgatctggcctcagacgggtgcccttgagagaagcagagggagtggcatgcagggcccctgccatgggtgcgctcctcaccggagcaaagcagcatgataaggactgcagcgggggagctctggggagcagcttgtgtagacaagcgcgtgctcgctgagccctgcaaggcagaaatgacagtgcaaggaggaaatgcagggaaactcccgaggtccagagccccacctcctaacaccatggattcaaagtgctcagggaatttgcctctccttgccccattcctggccagtttcacaatctagctcgacagagcatgaggcccctgcctcttctgtcattgttcaaaggtgggaagagagcctggaaaagaaccaggcctggaaaagaaccagaaggaggctgggcagaaccagaacaacctgcacttctgccaaggccagggccagcaggacggcaggactctagggaggggtgtggcctgcagctcattcccagccagggcaactgcctgacgttgcacgatttcagcttcattcctctgatagaacaaagcgaaatgcaggtccaccagggagggagacacacaagccttttctgcaggcaggagtttcagaccctatcctgagaatggggtttgaaaggaaggtgagggctgtggcccctggacgggtacaataacacactgtactgatgtcacaactttgcaagctctgccttgggttcagcccatctgggctcaaattccagcctcaccactcacaagctgtgtgacttcaaacaaatgaaatcagtgcccagaacctcggtttcctcatctgtaatgtggggatcataacacctacctcatggagttgtggtgaagatgaaatgaagtcatgtctttaaagtgcttaatagtgcctggtacatgggcagtgcccaataaacggtagctatttaaaaaaaaaaaaaaaaaaaaaaaaaa

SEQ ID NO: 5 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 6 - DNA, 1147 bp, Homo sapiens
cgaccaggttctccttccccagtcaccagttgctcgagttagaattgtctgcaatggccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatttgaccagagcaaagctgaaaaatgaataactaaccccctttccctgctagaaataacaattagatgccccaaagcgattttttttaaccaaaaggaagatgggaagccaaactccatcatgatgggtggattccaaatgaacccctgcgttagttacaaaggaaaccaatgccacttttgtttataagaccagaaggtagactttctaagcatagatatttattgataacatttcattgtaactggtgttctatacacagaaaacaatttattttttaaataattgtctttttccataaaaaagattactttccattcctttaggggaaaaaacccctaaatagcttcatgtttccataatcagtactttatatttataaatgtatttattattattataagactgcattttatttatatcattttattaatatggatttatttatagaaacatcattcgatattgctacttgagtgtaaggctaatattgatatttatgacaataattatagagctataacatgtttatttgacctcaataaacacttggatatcc

SEQ ID NO: 7 - protein, 176 aa, Homo sapiens
MKASSLAFSLLSAAFYLLWTPSTGLKTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 8 - DNA, 1252 bp, Homo sapiens
ctttgaattcctagctcctgtggtctccagatttcaggcctaagatgaaagcctctagtcttgccttcagccttctctctgctgcgttttatctcctatggactccttccactggactgaagacactcaatttgggaagctgtgtgatcgccacaaaccttcaggaaatacgaaatggattttctgagatacggggcagtgtgcaagccaaagatggaaacattgacatcagaatcttaaggaggactgagtctttgcaagacacaaagcctgcgaatcgatgctgcctcctgcgccatttgctaagactctatctggacagggtatttaaaaactaccagacccctgaccattatactctccggaagatcagcagcctcgccaattcctttcttaccatcaagaaggacctccggctctgtcatgcccacatgacatgccattgtggggaggaagcaatgaagaaatacagccagattctgagtcactttgaaaagctggaacctcaggcagcagttgtgaaggctttgggggaactagacattcttctgcaatggatggaggagacagaataggaggaaagtgatgctgctgctaagaatattcgaggtcaagagctccagtcttcaatacctgcagaggaggcatgaccccaaaccaccatctctttactgtactagtcttgtgctggtcacagtgtatcttatttatgcattacttgcttccttgcatgattgtctttatgcatccccaatcttaattgagaccatacttgtataagatttttgtaatatctttctgctattggatatatttattagttaatatatttatttattttttgctatttaatgtatttatttttttacttggacatgaaactttaaaaaaattcacagattatatttataacctgactagagcaggtgatgtatttttatacagtaaaaaaaaaaaaccttgtaaattctagaagagtggctaggggggttattcatttgtattcaactaaggacatatttactcatgctgatgctctgtgagatatttgaaattgaaccaatgactacttaggatgggttgtggaataagttttgatgtggaattgcacatctaccttacaattactgaccatccccagtagactccccagtcccataattgtgtatcttccagccaggaatcctacacggccagcatgtatttctacaaataaagttttctttgcataacaaaaaaaaaaaaaaaaaaa

SEQ ID NO: 9 - protein, 207 aa, Homo sapiens
MNFQQRLQSLWTLASRPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 10 - DNA, 1979 bp, Homo sapiens
gcttgcctgcaaacctttacttctgaaatgacttccacggctgggacgggaaccttccacccacagctatgcctctgattggtgaatggtgaaggtgcctgtctaacttttctgtaaaaagaaccagctgcctccaggcagccagccctcaagcatcacttacaggaccagagggacaagacatgactgtgatgaggagctgctttcgccaatttaacaccaagaagaattgaggctgcttgggaggaaggccaggaggaacacgagactgagagatgaattttcaacagaggctgcaaagcctgtggactttagccagcagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctgaatgtctagaccaggacctccctccccctggcactggtttgttccctgtgtcatttcaaacagtctcccttcctatgctgttcactggacacttcacgcccttggccatgggtcccattcttggcccaggattattgtcaaagaagtcattctttaagcagcgccagtgacagtcagggaaggtgcctctggatgctgtgaagagtctacagagaagattcttgtatttattacaactctatttaattaatgtcagtatttcaactgaagttctatttatttgtgagactgtaagttacatgaaggcagcagaatattgtgccccatgcttctttacccctcacaatccttgccacagtgtggggcagtggatgggtgcttagtaagtacttaataaactgtggtgctttttttggcctgtctttggattgttaaaaaacagagagggatgcttggatgtaaaactgaacttcagagcatgaaaatcacactgtcttctgatatctgcagggacagagcattggggtgggggtaaggtgcatctgtttgaaaagtaaacgataaaatgtggattaaagtgcccagcacaaagcagatcctcaataaacatttcatttcccacccacactcgccagctcaccccatcatccctttcccttggtgccctccttttttttttatcctagtcattcttccctaatcttccacttgagtgtcaagctgaccttgctgatggtgacattgcacctggatgtactatccaatctgtgatgacattccctgctaataaaagacaacataactcaagtctggcagactttcttctctatttctggatgaatgcccagtgagactgtgttgtacagctagaaaaggccttcttcccaatagcaaggctgtgcatctagcctcaagctctggctgaactttgtggtcgacatcaatctaaagatacagtgtctgactataaccttgttccaaaaacctaggcaaagagtatatgtaggaggtgggatatcacttccatgacataagtgctattgcagagccgtggccacccaggaactcctgactgctttcc

SEQ ID NO: 11 - protein, 263 aa, Homo sapiens
MMPKHCFLGFLISFFLTGVAGTQSTHESLKPQRVQFQSRNFHNILQWQPGRALTGNSSVYFVQYKIMFSCSMKSSHQKPSGCWQHISCNFPGCRTLAKYGQRQWKNKEDCWGTQELSCDLTSETSDIQEPYYGRVRAASAGSYSEWSMTPRFTPWWETKIDPPVMNITQVNGSLLVILHAPNLPYRYQKEKNVSIEDYYELLYRVFIINNSLEKEQKVYEGAHRAVEIEALTPHSSYCVVAEIYQPMLDRRSQRSEERCVEIP